(19) United States
(12) Patent Application Publication
(10) Pub. No.: US 2024/0156872 A1
(43) Pub. Date: May 16, 2024

(54) Use of Chaperone-Mediated Autophagy Activators For Treating Or Preventing Blood Cancers And Myelodysplastic Syndromes And Enriching Hematopoietic Stem Cell Populations

(71) Applicant: ALBERT EINSTEIN COLLEGE OF MEDICINE, Bronx, NY (US)

(72) Inventors: Ana Maria CUERVO, Bronx, NY (US); Evripidis GAVATHIOTIS, Flushing, NY (US)

(21) Appl. No.: 18/351,655

(22) Filed: Jul. 13, 2023

Related U.S. Application Data
(63) Continuation of application No. PCT/US2022/012309, filed on Jan. 13, 2022.
Application No. PCT/US2022/012309 is a continuation of application No. PCT/US2022/039959, filed on Aug. 10, 2022.
(60) Provisional application No. 63/136,974, filed on Jan. 13, 2021.
(60) Provisional application No. 63/231,476, filed on Aug. 10, 2021.

(51) Int. Cl. A61K 35/28; A61K 9/00; A61K 31/421; A61K 31/497; A61K 31/498; A61K 31/538; C12N 5/0789

(52) U.S. Cl. CPC A61K 35/28; A61K 9/0053; A61K 31/421; A61K 31/497; A61K 31/498; A61K 31/538; C12N 5/0647

(57) ABSTRACT

The present disclosure provides methods of treating disease and disorders of the hematological system. The disclosure provides a method of expanding a population of hematopoietic stem cells (HSCs) while maintaining the population in an undifferentiated state comprising contacting the population of HSCs in the presence of a CMA activator for a period of time until a sufficient number of HSCs is obtained thereby producing an expanded HSC population.

### CROSS REFERENCE TO RELATED APPLICATION

This application is a continuation filed under 35 USC § 111(a) of international application PCT/US22/12309, filed Jan. 13, 2022, which claims priority of U.S. Provisional Appl. No. 63/136,974, filed Jan. 13, 2021, and also of international application PCT/US22/39959, which claims priority of U.S. Provisional Appl. No. 63/231,476 filed Aug. 10, 2021, and all the benefits accruing therefrom under 35 USC § 119, each of which is hereby incorporated by reference in its entirety.

### STATEMENT OF GOVERNMENT SUPPORT

This invention was made with government support under AG021904, AG038072, AG031782, DK105134, CA230756, HL146442, HL149714, HL148151 and HL150032 awarded by the National Institutes of Health. The U.S. Government has certain rights in this invention.

### FIELD OF THE INVENTION

The present invention relates generally to methods of using activators of chaperone-mediated autophagy (CMA) to treat various hematological and extra-hematological diseases, such as cancer. The invention also provides methods of expanding hematopoietic stem cells (HSC) using activators of CMA.

### BACKGROUND OF THE INVENTION

Hematopoietic (blood-forming) stem cells (HSCs) produce all mammalian blood cells, including key immune cells that provide protection from bacteria and viruses. HSCs harbor extensive self-renewal and multilineage differentiation capabilities to sustain production of blood cells over a lifetime. These adult stem cells are mostly quiescent, but intrinsic and extrinsic cues signal HSC to enter the cell cycle and self-renew or differentiate. Maintenance of a fully functional proteome that can rapidly change to facilitate the transition of HSC from quiescence to activation and again return to quiescence, is essential to prevent stem cell exhaustion and bone marrow failure.

As mammals age, HSCs become less efficient and less able to make healthy new blood cells. Repeated activation of HSC and exposure to cellular insults results in compromised HSC function with age in the form of severely reduced self-renewal and multilineage repopulation capacities. This functional decline of HSC in aging has been attributed to increasing levels of reactive oxygen species (ROS), accumulation of DNA damage and declining proteostasis. Macroautophagy, the most studied type of autophagy, contributes to maintenance of HSC quiescence through mitochondria degradation to limit oxidative phosphorylation. However, the mechanisms governing cellular reprograming upon stem cell activation and their subsequent return to quiescence are still not fully characterized.

Applicants have found that this reduction in HSC efficiency is caused in part by the deterioration of chaperone-mediated autophagy (CMA), the housekeeping process that removes damaged proteins and other waste materials that interfere with cells' ability to function. There is need for developing effective methods of preventing stem cell exhaustion and bone marrow failure. The invention fulfills this need.

### SUMMARY OF THE INVENTION

The disclosure includes a method of expanding a population of hematopoietic stem cells (HSCs) while maintaining the population in an undifferentiated state comprising culturing the population of HSCs in the presence of a chaperone mediated autophagy (CMA) activator for a period of time until a sufficient number of HSCs is obtained thereby producing an expanded HSC population. In some embodiment the expanded HSC population expresses lysosome-associated membrane glycoprotein 2 isoform A (L2A) or human L2A (hL2A).

The disclosure further includes a method for suppressing the differentiation of a hematopoietic stem cell (HSC) comprising contacting the HSC with a CMA activator. The CMA can be administered to a subject, for example prior to, contemporaneously with, or after, the subject receives an HSC transplant.

The disclosure includes a method for treating or preventing a disease or disorder resulting from a deficiency or dysfunction of hematopoietic stem cells (HSCs), comprising administering to a subject in need thereof an effective amount of a CMA activator and/or the expanded HSC population provided by the disclosure. The disclosure also provides a method of treating or preventing a disease of disorder of the hematopoietic system comprising administering to a subject in need thereof an effective amount of a CMA activator and or the expanded HSC population of the disclosure. The administration of the expanded HSC population can be by any pharmaceutically acceptable route, for example enteral or parental, including intravenous administration. The administration of the CMA activator can be by any acceptable route, for example the CMA activator can be administered orally.

The method of this disclosure can be used to treat or prevent a disease or disorder in a patient where the disease or disorder responsive to suppressing the differentiation of the patient's hematopoietic stem cells or alleviating a deficiency or dysfunction of the patient's hematopoietic stem cells. In certain embodiments the disease or disorder is a red blood cell disorder, anemia, congenital dyserythropoietic anemia, congenital sideroblastic anemia, G6PD deficiency, hemochromatosis, hemolytic anemia, hemolytic disease of the newborn, hydrops fetalis, iron deficiency anemia, iron-refractory iron deficiency anemia (IRIDA syndrome), megaloblastic anemia (including pernicious anemia), pyruvate kinase PK) deficiency, sickle cell disease, spherocytosis, thalassemia, white blood cell disorders, cyclic neutropenia, severe congenital neutropenia (Kostmann syndrome), chronic granulomatous disease, leukocyte adhesion deficiency, myeloperoxidase deficiency, bone marrow failure syndromes, aplastic anemia, congenital amegakaryocytic thrombocytopenia, Diamond-Blackfan anemia, dyskeratosis congenita, Fanconi anemia, myelodysplastic syndrome (MDS), Pearson syndrome, Shwachman-Diamond syndrome, thrombocytopenia absent radius, bleeding disorders, hemophilia, hypofibrinogenemia and dysfibrinogenemia, platelet function disorders thrombocytopenia, Von Willebrand disease, thrombosis, anticoagulation disorders, antithrombin deficiency, factor V Leiden, protein C deficiency, protein S deficiency, prothrombin gene mutation, stroke, thrombosis, autoimmune blood cell disorders, autoimmune hemolytic anemia, Evans syndrome, immune thrombocytopenia (IPT), a myeloproliferative neoplasm (MPN), or polycythemia. The MPN can be polycythemia vera (PV), essential thrombocythemia (ET), myelofibrosis, juvenile myleomonocytic leukemia (JMML), chronic neutrophilic Leukemia, or chronic eosinophilic leukemia/hypereosinophilic syndrome (HES), mast cell disease, chronic myelogenous leukemia, or acute myeloid leukemia (AML). The myelofibrosis can be primary myelofibrosis (PMF).

The disclosure includes a method of expanding a population of blood cells comprising culturing the population of blood cells in the presence of a CMA activator for a period of time until a significant increase in the number of blood cells is obtained, thereby expanding the population of blood cells. The blood cell can be a red blood cell or a white blood cell, for example a leukocyte. The leukocyte can be a lymphocyte, for example a B-cell or a T-cell. The T-cell can be a CAR-T cell.

The disclosure provides a method of increasing the activity of a population of hematopoietic (blood-forming) stem cells (HSCs) in vivo or in vitro, the method comprising contacting the population of HSCs with a sufficient concentration of a Chaperone-mediated Autophagy (CMA) activating compound to increase the number of blood cells produced by the HSCs in a time period relative to the number of blood cells produced by the HSCs in the same time period prior to being contacted with the CMA. The disclosure also includes a method of improving the proteostasis of a population of hematopoietic (blood-forming) stem cells (HSCs) in vivo or in vitro comprising contacting the population of HSCs with a sufficient concentration of a Chaperone-mediated Autophagy (CMA) activating compound to sufficient to improve a marker of proteostasis in the population of HSCs relative to the marker of proteostasis in the population of HSCs prior to being contacted with the CMA.

The disclosure provides a method of increasing the activity of a population of hematopoietic (blood-forming) stem cells (HSCs) in vivo or in vitro comprising contacting the population of HSCs with a sufficient concentration of a Chaperone-mediated Autophagy (CMA) activating compound to increase the number of blood cells produced by the HSCs in a time period relative to the number of blood cells produced by the HSCs in the same time period prior to being contacted with the CMA.

The disclosure also includes a method of treating or reducing the risk of a blood cancer or treating or reducing the risk of myelodysplastic syndrome in a mammal comprising administering a therapeutically effective amount of a CMA activator to the mammal.

The disclosure includes a method of improving the proteostasis of a population of hematopoietic (blood-forming) stem cells (HSCs) in vivo or in vitro comprising contacting the population of HSCs with a sufficient concentration of a Chaperone-mediated Autophagy (CMA) activating compound to sufficient to improve a marker of proteostasis in the population of HSCs relative to the marker of proteostasis in the population of HSCs prior to being contacted with the CMA. The marker of proteostasis can be glycolysis, fatty acid metabolism, compound, polyunsaturated fatty acid level, or number of activated T cells, each of which is increased in HSCs contacted with the CMA activating compound.

The disclosure includes any of the above methods, wherein the CMA activator is a compound of Formula I, Formula II, or Formula III or a pharmaceutically acceptable salt thereof. The disclosure includes any of the above methods, wherein the CMA activator is a compound of Table 1, Table 2, or Table 3, or a pharmaceutically acceptable salt thereof.

### BRIEF DESCRIPTION OF THE DRAWINGS

FIG. 1. CMA activity is required for HSC self-renewal. FIG. 1a, Dendra fluorescence in sorted HSC from 4 m, 12 m and 30 m old KFERQ-Dendra mice. FIG. 1b, Quantification of Dendra puncta per cell in a. n=9 fields from 4 individual mice (individual points represent the average of approx. 7-10 cells per field). FIG. 1c, Dendra fluorescence in HSC and myeloid progenitors (Lin−cKit+Sca-1—) from KFERQ-Dendra mice before and at the indicated days after single injection of 5-fluorouracil (5FU). Right: quantification of Dendra+ puncta per cell in FIG. 1c. n=9 fields from 5 individual mice (individual points represent the average of 7-10 cells per field). FIG. 1d, HSC frequency in BM cells from Ctrl and L2AKO mice untreated (Basal) or 8 days after a single injection with 5-fluorouracil (5FU). n=6 mice. FIG. 1e, Survival curve of Ctrl and L2AKO mice after serial injections of 5FU 7 days apart. FIG. 1f, White blood cell counts 7 days after first (left) and second (right) 5FU injection. n=8 (Ctrl) and 10 (L2AKO) mice. FIG. 1g-h, Serial transplantation and competitive BM repopulation with Ctrl or L2AKO BM cells, experimental strategy (FIG. 1g upper); frequency of donor-derived HSC 16 weeks after the first competitive transplantation n=5 mice (FIG. 1h, lower) and FIG. 1i, donor cell (from 3-4 m CD45.2 mice) contribution in recipients' peripheral blood 4 or 16 weeks after competitive secondary transplantation. n=7 mice . FIG. 1j, Serial colony formation assay with HSC from Ctrl or L2AKO mice. Number of colonies at day 10 after the indicated plating number is shown. n=6 (1st plating) and 4 (2nd and 3rd plating) mice. FIG. 1j, LTC-IC assay of LSK cells from 4 m Ctrl and L2AKO mice. Fold change of LTC-IC frequency relative to Ctrl after 4 weeks of culture is shown, n=3 mice. One-way ANOVA test followed by Tukey's multiple comparison post-hoc test (FIG. 1b, FIG. 1c) and two-way ANOVA test followed by Sidak's multiple comparison post-hoc test (FIG. 1f,h,i), Log-rank (Mantel-Cox) test (FIG. 1e) and unpaired t-test (FIG. 1d,g,j) were used. P<0.05 (*), 0.01 (**), 0.0001(****). ns: no statistical significance.

FIG. 2. Consequences of CMA blockage on HSC function. a-c, FIG. 2a, Percentage of stem cells in active cell cycle (not in GO by Ki67 and Hoechst staining), FIG. 2b, ATP levels and FIG. 2c, median fluorescence intensity (MFI) for cellular ROS (reactive oxygen species) in HSC from control (Ctrl) and L2AKO mice untreated (basal) or at day 8 after a single 5FU injection. n=6-7 mice (FIG. 2a), 4-5 mice (FIG. 2b), 5-15 (FIG. 2c) mice. Representative examples of FACS plots for FIG. 2a and cycling analysis by Brdu incorporation are shown in FIG. 8a-c. FIG. 2d, ROS levels in Ctrl and L2AKO bone marrow (BM) derived HSC in transplanted recipients. n=5 mice. FIG. 2e, and enrichment pathway analysis (FIG. 2e) in HSC cells from Ctrl and L2AKO mice untreated or 8 days post 5FU injection. FIG. 2f, STRING analysis of proteins whose levels decrease in control cells upon activation (top) and those at higher levels in L2AKO LSK than Ctrl LSK 8 days post-5FU (bottom). FIG. 2g, Number of proteins different and overlapping in the same two groups as in g. n=3 proteomic experiments with pool of 3 mice per group. FIG. 2h-j, Metabolic phenotypes of LSK cells from Ctrl and L2AKO mice. Unsupervised principal component analysis of the two groups (FIG. 2h), hierarchical clustering analysis of the top 25 significant metabolites by two-tailed t test (FIG. 2i) and Omicsnet analysis of the most significantly affected pathways in L2AKO cells (FIG. 2j). n=3 metabolomic experiments with pool of 3 mice per group. FIG. 2k, Extracellular acidification rates (ECAR) in Ctrl and L2AKO LSK cells and changes upon addition of glucose (Glu), oligomycin (Oligo) and 2-Deoxy-D-glucose (2DG). n=3 independent experiments. FIG. 2I, Basal glycolysis (left), glycolytic capacity (middle) and glycolytic reserve (right) in L2AKO LSK cells relative to control cells. n=3 independent experiments. FIG. 2m. GAPDH (left) and pyruvate kinase (PK) (right) activity in Ctrl and L2AKO LSK cells. n=3 independent experiments. FIG. 2n, Percentage of cellular oxidized GAPDH and PK detected by mass spec in Ctrl and L2AKO LSK cells under basal conditions. n=9 mice in 3 different experiments. FIG. 2o, Percentage of total cellular proteins that are oxidized (left) and fold changes in the number of carbonylated peptides (right) in LSK from Ctrl and L2AKO mice under basal conditions or 8 days after 5FU injection. n=9 mice in 3 independent experiments. FIG. 2p,q Oxidized proteins (FIG. 2p) and protein inclusions (FIG. 2p) in basal Ctrl and L2AKO HSC detected by staining with OxylCC and Proteostat, respectively. Representative images (left) and quantification of staining intensity (right) are shown. n=15 fields from 5 individual mice (individual points represent the average of 5-10 cells per field). Nuclei are highlighted with DAPI. Full field images for FIG. p and q as shown in FIG. 10g. Two-way ANOVA test followed by Sidak's (FIG. 2a-c) or Tukey's (FIG. 2p) multiple comparison post-hoc test, Chi-square test (FIG. 2i), multiple time point paired t-test (I) and unpaired t-test (FIG. 2d, 1-n, p, q) were used for the statistics. P<0.05 (*), 0.01 (**), 0.001(***), 0.0001 (****). ns: no statistical significance.

FIG. 3. CMA increases linoleic acid metabolism upon HSC activation. FIG. 3a, Top lipid metabolism pathways identified from enrichment analysis of metabolites as different between control and L2AKO LSK cells 8 days post 5FU injection. FIG. 3b, Heat map of substrate and metabolite abundance in the linoleic acid and α-linolenic metabolism in the indicated conditions. FIG. 3c, Scheme of linoleic and α-linolenic fatty acid metabolism pathway showing the increased (up arrow) and decreased (down arrow) metabolites in L2AKO LSK cells comparing with Ctrl cells in 5FU-activated conditions. FIG. 3d, Levels of substrates (bars 1-2 for each data set) and downstream metabolites (bars 2-6 for each data set) of the linoleic acid and α-linolenic metabolism relative to those in unstimulated control cells. n=9 mice in 3 different experiments. Significant differences with control untreated are marked with * and between L2AKO basal and 5FU treated with †. FIG. 3e, Total colony numbers (left) and number of immature, multipotent colony-forming unit-granulocyte/erythroid/macrophage/megakaryocyte CFU-GEMM (right) in the first plating from Ctrl and L2AKO HSC treated or not with the FADS2 inhibitor SC-26196 (SC). n=3 independent experiments. FIG. 3f, Total colony numbers in the 3rd plating (left) or number of CFU-GEMM colonies in the first plating (right) from Ctrl and L2AKO HSC treated or not with γ-linolenic acid (GLA). n=4 independent experiments. FIG. 3g, Predicted CMA-targeting motif in FADS2 (MDRK) generated by acetylation of 42K (g top) and analysis of the acetylation level of this peptide detected by mass spectrometry Ctrl and L2AKO LSK cells in basal and 5FU activated conditions (FIG. 3g bottom). n=3 mice. FIG. 3h,i, Immunoblot for Flag of Ctrl (h) and L2AKO (i) ex vivo expanded HSC expressing Flag-myc tagged FADS2 wild type (VIDRK) or mutated as indicated at the top (VIDRQ or VIDRA) treated or not with NH4Cl/Leupeptin (N/L). For gel source data, see supplementary FIG. 1. FIG. 3j, Oxygen consumption rate (OCR) in Ctrl and L2AKO LSK cells at day 8 after 5FU injection. Responses to addition of oligomycin (Oligo), FCCP and rotenone (Roteno) are shown. n=5 independent experiments. FIG. 3k, Quantification of mitochondrial respiration from fatty acid β-oxidation (etomoxir-sensitive) in Ctrl and L2AKO LSK cells. n=5 independent experiments. FIG. 31, Total colony numbers in 3rd plating from both Ctrl and L2AKO HSC treated or not with Methyl-pyruvate (5mM) starting from the 1st plating. n=7 (None) and 4 (Methyl-pyruvate) independent mice. m, Quantification of MFI of ROS+ cells (left) and total colony numbers (right) in the 3rd plating of Ctrl or L2AKO HSC untreated or supplemented with NAC from the 1st plating. n=7 (None) and 4 (NAC) mice. FIG. 3n, Ratio of y-linolenic acid (GLA) and linoleic acid (LA) in LSK cells from young and old mice calculated from the metabolomics data. n=9 mice in 3 different experiments. FIG. 3o, Ratio of K42 acetylated and total FADS2 peptide in LSK from young and old mice calculated from the mass spectrometry analysis. n=9 mice in 3 different experiments. FIG. 3p, Total colony numbers (left) and number of CFU-GEMM colonies (right) in the colony formation assay with HSC cells from old (22 m) mice daily injected with either saline or GLA (1mg/kg bw) for 7 weeks. Representative images of wells with Ctrl or GLA treated cells are shown on the top. n=5 mice per group. q, LTC-IC assay according to GEMM colonies from Ctrl and GLA treated human CD34+ cells from old (>65 year) multi-myeloma patients. n=2 patients. Two-way ANOVA test followed by Tukey's (FIG. 3d,f) or Sidak (FIG. 3e,l,m) multiple comparison post-hoc test, unpaired t-tests (FIG. 3g,k,n,o,p), time point paired t-test (FIG. 3j), and Chi-square test (FIG. 3q) were used for the statistics. P<0.05 (*), 0.01 (**), 0.001(***), 0.0001(****). ns: no statistical significance.

FIG. 4. Modulation of CMA restores old HSC function. FIG. 4a, Changes in HSC numbers per femur and tibia in Ctrl and L2AKO mice with age. Values are relative to young control mice. n=9-11 mice. FIG. 4b, Donor chimerism at the indicated time points in peripheral blood of recipients after competitive transplantation of 25-30 m Ctrl or L2AKO BM cells. n=5-6 mice. FIG. 4c, Donor lineage distribution in peripheral blood 24 weeks after second competitive transplantation of Ctrl or L2AKO BM cells. n=5-6 mice. FIG. 4d, ROS levels in HSC from 3-4m and >25m old Ctrl and L2AKO mice. Data is shown as median fluorescence intensity (MFI) per cell. n=5-6 mice. FIG. 4e, Percentage of HSC in BM of young, old control and old mice bearing an extra copy of human L2A (hL2AOE). n=5 (young), 18 (old ctrl) and 13 (old hL2AOE) mice. FIG. 4f,g, Representative FACS (FIG. 4f) and quantification (g) of ROS levels in HSC cells from young, old Ctrl and hL2AOE mice. Data is shown relative to old Ctrl mice. n=5 mice. FIG. 4h-j PK (FIG. 4h) and GAPDH (FIG. 4i) activity and extracellular acidification rates (ECAR) in basal conditions or after addition of oligomycin (Oligo) and 2-deoxy-D-glucose (2DG) (FIG. 4j) in LSK cells from old Ctrl and hL2AOE mice. n=3 mice. FIG. 4k Levels of polyunsaturated fatty acids generated by FADS2 in old Ctrl and hL2AOE mice expressed as peak area relative to young mice. n=9 (young), 18 (old), 9 (hL2AOE) mice in 3 independent experiments. FIG. 41. Percentage of donor derived cells at the indicated times in mice transplanted with BM from old Ctrl and hL2AOE mice. n=5 mice. m-o, Levels of oxidized proteins (FIG. 4m), GAPDH activity (FIG. 4n) and ECAR in basal conditions or upon Oligo and 2DG addition (FIG. 4o) in HSC from old (>22m) mice 2 months after daily oral administration of a chemical activator of CMA (CA 20 mg/Kg b.w.) or the corresponding vehicle (Veh.). Values in young mice are shown as reference in n. Representative images of cells used for quantification in m are in FIG. 13n. n=12 fields from 4 and 3 mice for FIG. 4m and FIG. 4n, respectively. p. LTC-IC assay of LSK cells from BM of old mice administered daily for 2 months CA or vehicle. Scatter plot shows LTC-IC frequency for vehicle and CA treated old mice. n=4 mice. FIG. 4q,r LTC-IC assay of old LSK cells after ex vivo treatment with vehicle (DMSO) or a chemical activator of CMA (CA 10 μM, daily for 4 weeks) (FIG. 4q) and viable cell percentage recovered from the colonies formed at the end of LTC-IC (FIG. 4r). Individual plot showing the fold change of LTC-IC frequency compared to old control cells (FIG. 4q left), scatter plot showing LTC-IC frequency for vehicle and CA treated old Ctrl cells (FIG. 4q right). n=3 mice. FIG. 4s, Representative images of Giemsa staining of the cells recovered at the end of the LTC-IC assay when cells were plated in presence of CA (10p,M) or vehicle. n=3 experiments. Figs. t,u. Quantification of immature GEMM colonies in the 1st plating (FIG. 4t) and total colony numbers in the 2nd plating (FIG. 4u) of CD34+ enriched stem and progenitor cells from mobilized blood of multi-myeloma patients (59, 65, and 71 years old), when maintained in presence of CA (10 μM) or vehicle starting from the 1st plating. n=3 patients. Multiple t-tests (FIGS. 4a-c), unpaired t-tests (FIGS. 4m,q left, FIGS. 4r,t,u), time points paired t-test (FIG. 4j, o), two-way ANOVA test followed by Sidak's multiple comparisons post-hoc test (d, l) and one-way ANOVA tests followed by Tukey's (FIG. 4e,g-i,n), Sidak's (FIG. 4k) or Dunnett's (FIG. 4n) multiple comparisons post-hoc test and Chi-square test (FIG. 4p,q right) were used for the statistics. P<0.05 (*), 0.01 (**), 0.001 (***), 0.001(****). ns: no statistical significance.

FIG. 5. CMA changes in HSC with age. FIG. 5a, Scheme of the KFERQ-Dendra fluorescent CMA reporter. FIG. 5b, Percentage of HSC that are positive for Dendra puncta at the indicated ages. n=4 mice for each age (individual point represents an average of a mouse). Cells that have more than 2 Dendra+ puncta are defined as CMA+ cells. FIG. 5c, Dendra fluorescence in sorted GMP cells from young and old KFERQ-Dendra mice. Left: Representative images and magnified cells. Right: Quantification of puncta per cell. n=8 fields from 3 mice per age group (individual point represents an average of 7-10 cells in the field). FIG. 5d, Dendra fluorescence in sorted HSC from young and old L2AKO-KFERQ-Dendra mice. Left: Representative images and magnified cells. Right: Quantification of puncta per cell. n=8 fields from 4 mice per age group. FIG. 5e, Immunostaining for LAMP1 and LAMP2A in HSC from young and old mice. Top: Representative images of full fields and magnified cells. Bottom: Quantification of fluorescence intensity of each protein. n=8 (LAMP1), 11 (LAMP2A) fields from 3 mice per age group (individual point represents an average of 7-10 cells in the field). FIG. 5f, Percentage of proteins bearing KFERQ-like motifs among those proteins identified to be at higher (O>Y) and lower (O<Y) abundance in old human BM HSC compared to young ones (data set in ref 49). Motif frequency in the total proteome is shown as reference. HSC were purified from BM samples aspirated from 59 human subjects (45 male and 14 female) with age ranged from 20 to 60 years (with median of 33.2 years). Only proteins that displayed statistically significant (p<0.01) changes in levels with age when comparing the 59 subjects were included in the analysis of KFERQ-like motifs. Bars are mean+/−s.d. (or 95% confidence interval) estimated by bootstrapping. FIG. 5g, Cellular pathway enrichment STRING analysis of the proteins bearing CMA-targeting motifs that accumulate in old human BM HSC. One-way (FIG. 5b) or two-way (FIG. 5f) ANOVA with Tukey's multiple comparisons post-hoc test and unpaired t test (FIG. 5c,d,e) were used. P<0.01 (**), P<0.0001 (****). ns: no statistical significance.

FIG. 6. CMA upregulation during HSC activation. FIG. 6a, Left: Full fields of the images shown at higher magnification in FIG. 1c top. Right: Percentage of HSC that are positive for Dendra puncta at the indicated times. n=4 mice per group (individual point represents an average of cells in a mouse). Cells that have more than 2 Dendra+ puncta are defined as CMA+ cells. FIG. 6b, Lysosomal proteolysis of long-lived proteins in HSC untreated (Ctrl) and 8 days post 5FU injection. n=3 independent experiments. FIG. 6c, Immunostaining (top) and quantification (bottom) for LAMP2A, LAMP1 and LysoTracker in HSC from mice before and 1, 3 and 8 days after single 5FU injection. n=6-16 fields from 3 mice, each individual point represents an average of 7-10 cells per field. FIGS. 6d,e, Dendra fluorescence (FIG. 6d) and quantification of puncta per cell (FIG. 6e) in HSC isolated from KFERQ-Dendra mice 48 hours after injection of PolyI:C or vehicle control (Basal). n=3 mice. Individual point represents an average of 7-10 cells per field. FIGS. 6f,g, Full fields of the images shown at higher magnification in FIG. 1c bottom (FIG. 6f) and number of Dendra puncta per cell (FIG. 6g) in myeloid progenitors from KFERQ-Dendra mice before and at the indicated days after single injection of 5-fluorouracil (5FU). n=3 mice. FIGS. 6h,i, Dendra fluorescence in myeloid progenitors isolated from KFERQ-Dendra mice 48 hours after injection of PolyI:C or vehicle control (Basal) (FIG. 6h) and quantification of number of dendra puncta per cell (FIG. 6i). n=3 mice. Unpaired t tests (FIG. 6b, FIG. 6e, FIG. 6i) and one-way ANOVA test followed by Tukey's multiple comparisons post-hoc test (FIG. 6a, FIG. 6c, FIG. 6g) were used. P<0.05 (*), 0.01 (**). ns: no statistical significance.

FIG. 7. Effect of CMA blockage on HSC and myeloid progenitor cells. FIG. 7a, Quantitative PCR for the three lamp2 spliced variants in BM from ^{Vav-i}CreL2A^{f/f} (L2AKO) and V^{avi}Cre(Ctrl) mice. n=3 independent experiments. FIG. 7b, Number of total BM cells of 2 femurs and tibias from Ctrl and L2AKO mice in basal and at day 8 post 5FU injection. n=5 mice. FIG. 7c, Percentage of T cells (CD3ε+), B cells (B220+), granulocytes (CD11b⁺Grl^{high}) and monocytes (CD11b⁺ Grl^{middle}) in Ctrl and L2AKO mice BM in basal conditions. n=5 mice. FIG. 7d, Frequency of Lin−cells of 2 femurs and tibias from Ctrl and L2AKO mice in basal and day 8 post 5FU injection. n=4-6 mice. FIG. 7e,f, Number of LSK cells (FIG. 7e) and HSC cells (FIG. 7f) in BM of Ctrl and L2AKO mice in basal and 5FU activated conditions. n=6 mice. FIG. 7g,h, Donor chimerism in peripheral blood (FIG. 7g) and donor derived Lin- population (FIG. 7h) in BM of recipients at the indicated weeks after the primary competitive transplantation using BM from 4 m control and L2AKO mice. n=7 mice (FIG. 7g), n=5 mice (FIG. 7h). FIG. 7i-k, Donor lineage distribution in peripheral blood of recipients in the secondary competitive transplantation of Ctrl and L2AKO BM. Representative FACS plots (left) and quantification (right) are shown. n=7 mice. 1, LTC-IC frequency for both Ctrl and L2AKO cells in the LTC-IC assay of Lin-Sca-l+c-Kit+ (LSK) cells from Ctrl and L2AKO mice. n=3 mice. Two way ANOVA followed by Sidak's multiple comparisons post-hoc test (FIG. 7b-d), multiple t tests (FIG. 7g) and unpaired t tests (FIG. 7e,f, h-k) and Chi-square test (FIG. 7l) were used. P<0.05 (*), 0.001 (***), 0.0001(****). ns: no statistical significance.

FIG. 8. Impact of CMA blockage in the cell cycle of HSC and myeloid progenitor cells. FIG. 8a-c, Representative FACS plots of Hoechst and Ki67 staining (FIG. 8a) or Hoechst and BrdU staining (FIG. 8c) in HSC from control (Ctrl) and L2AKO mice untreated (basal) or 8 days post single 5FU injection. Quantification of FACS in a is shown in main FIG. 2a and quantification of FACS in c is shown in FIG. 8b. n=4 mice. FIG. 8d,e, Representative FACS plots of Ki67 and Hoechst staining (FIG. 8e) and quantification (FIG. 8d) of cycling HSC from control (Ctrl) and L2AKO mice untreated (basal) or 16 days post single 5FU injection. n=4 mice. FIG. 8f,g, Percentage of cycling cells (FIG. 8f) and representative flow cytometry plots (g) of Ki67 and Hoechst-stained myeloid progenitors from Ctrl and L2AKO mice in basal conditions (top) and 8 days after a single 5FU injection (bottom). n=7 mice per condition. FIG. 8h,i, ATP (FIG. 8h) and ROS levels (FIG. 8i) in myeloid progenitors from Ctrl and L2AKO mice in basal or after single 5FU injection. Values in i are relative to Ctrl mice in basal conditions. n=4 (basal) and 3 (5FU) mice for FIG. 8h, n=5 mice for i. Two-way ANOVA tests followed by Sidak's multiple comparisons post-hoc test (FIG. 8b,f,h,i) and unpaired t test (FIG. 8d) were used. P<0.01 (**), 0.0001(****). ns: no statistical significance.

FIG. 9. Characterization of macroautophagy in CMA-deficient HSC cells. FIG. 9a, Immunostaining for endogenous LC3 in HSC from control (Ctr) and L2AKO mice in basal and cytokine starved conditions in presence or not of lysosomal inhibitors leupeptin and NH₄C1 (N/L). Representative images of full fields and higher magnification cells (left) and quantification of LC3 flux (right) calculated as the increase in number of LC3-positive puncta upon addition of N/L. n=9 fields from 3 mice (individual points represent average of 7-10 cells per field). FIG. 9b, Representative electron microscopy images of Ctrl and L2AKO HSC. Top: whole fields and boxed areas at higher magnification to show examples of autophagosomes (APG, arrows) and autolysosomes (AUT, arrows). Bottom: representative images of ultrastructure of the endoplasmic reticulum arrows) in Ctrl and L2AKO HSC under basal and 5FU (day8) activated conditions. FIG. 9c-e, Average number of APG , AUT (FIG. 9c) and mitochondrial number per cell (FIG. 9d) as well as cytoplasm and total cell area (FIG. 9e) from electron microscopy images in FIG. 9c. n=12-15 fields (FIG. 9c), 11 fields (FIGS. 9d[[9]]) and 6 fields (FIG. 9e) were quantified. FIG. 9f, Quantification of the average ER diameter in Ctrl and L2AKO HSC under basal and 5FU (day 8) activated conditions. n=8-11 fields. Two-way ANOVA test followed by Tukey's multiple comparisons post-hoc test (FIG. 9a) and unpaired t test (FIG. 9c-f) were used. P<0.05 (*), P<0.01 (**), P<0.0001 (****). ns: no statistical significance.

FIG. 10. Metabolism-related changes in the transcriptome and proteome of CMA-deficient HSC. FIG. 10a, Top categories of proteins related with overall metabolism (left) and break down of the proteins under enzyme category (right) found in the overlapped proteins in FIG. 6h. FIG. 10b, Scheme of glycolysis-related substrates and metabolites to highlight those significantly increased (red) or decreased (green) in L2AKO LSK when compared to control LSK. FIG. 10c, Changes in extracellular acidification rate (ECAR) (surrogate of glycolytic activity) in Ctrl and L2AKO myeloid progenitors upon addition of glucose (Glu), oligomycin (Oligo) and 2-Deoxy-D-glucose (2DG). FIG. 10d, Quantification of basal glycolysis (left), glycolytic capacity (middle) and glycolytic reserve (right) in Ctrl and L2AKO myeloid progenitors according to ECAR in FIG. 10c. Values are relative to those in control cells. n=3 independent experiments. FIG. 10e, Full field images of the images shown in FIG. 2p corresponding to HSC stained with OxyICC or Proteostat. Bottom panel shows full field and higher magnification inset of the 4-HNE staining for the same cells. FIG. 10f, Quantification of 4-HNE staining shown in FIG. 10e. n=8 (Ctrl) and 11 (L2AKO) fields from 3 mice. Individual points represent average of 7-10 cells per field. FIG. 10g, FIG. 10h, GAPDH (left) and PK (right) activity in 5FU-activated Ctrl and L2AKO LSK cells. n=3 independent experiments. Multiple t test by time segment (FIG. 10c) and unpaired t tests (FIG. 10c,g,h) were used. P<0.01 (**), 0.001 (***). ns: no statistical significance.

FIG. 11. Regulation of lipid metabolism by CMA during HSC activation. FIG. 11a, Metabolic pathways and functional impact identified as changing in the comparative metabolomic analysis of Ctrl and L2AKO LSK 8 days after a single 5FU injection. n =9 mice in 3 independent experiments. FIG. 11b, Immunostaining for FADS2 in LSK cells from Ctrl and L2AKO mice in the indicated conditions. Average of intensity per cell. n=16-23 fields from 3 independent experiments (FIG. 11b). Individual point represents an average of 7-10 cells per field. FIG. 11c, In silico analysis of proteins bearing phosphorylation- or acetylation-generated KFERQ-like motifs in the total proteome, the group of proteins at lower (L2AKO<Ctrl) and higher (L2AKO>Ctrl) abundance in L2AKO LSK cells upon 5FU activation (day 8). Left: Quantification of the percentage of proteins bearing the indicated types of motif in the groups. Right: STRING analysis of the enriched pathways of proteins bearing acetylation-generated KFERQ motif in the L2AKO>Ctrl group. FIG. 11d, Mass spectrometry analysis of acetylation-generated KFERQ motifs in the group of proteins with lower (L2AKO<Ctrl) and higher (L2AKO>Ctrl) acetylation in L2AKO LSK under 5FU activated condition. Left: Quantification of percentage of proteins with or without acetylation generated KFERQ motifs in each group. Right: STRING analysis of the enriched pathways of proteins with acetylation generated motifs in the group of proteins with higher acetylation (L2AKO>Ctrl). n=9 mice in 3 independent experiments. FIG. lle, Number of different and overlapping proteins with higher abundance in L2AKO LSK (L2AKO>Ctrl) and in old LSK (Old>Young) comparing with the young LSK. n=3 proteomic experiments with pool of 3 mice per group each. Bottom: STRING analysis of the enriched pathways in the group of proteins that accumulate in LSK from both L2AKO and old mice. FIGS. 11f-g. Metabolomic analysis in young (4 m) and old (>25 m) LSK cells in steady-state condition. Unsupervised principal component analysis (FIG. 11f). Comparison of metabolites in glycolysis (FIG. 11g) and in the step in linoleic acid metabolism catalyzed by FADS2 (FIG. 11h). Y axis represents integrated peak areas (arbitrary units) for each metabolite. n=9 mice in 3 different experiments. Two-way ANOVA tests followed by Tukey's (FIG. 11c,d) or Sidak's (c) multiple comparisons post-hoc test and unpaired t tests (FIGS. 11g,h) were used. P<0.05 (*), 0.01 (**), 0.0001 (****). ns: no statistical significance.

FIG. 12. CMA activity in hematopoietic stem cells from old mice. FIG. 12a, Quantification of KFERQ-dendra puncta per cell in HSC from 4, 12 and 30 m KFERQ-Dendra mice. Individual point in the plot represents a cell. n=45 (4 m), 52 (12 m) and 53 (30 m) cells from 4 mice per group. Cells with less than 5 puncta are shown in red. FIG. 12b, Representative images of full field (left) and higher magnification insets (right) of HSC from 30 m old mice after immunostaining for Dendra. Two different exposure times are shown for those cells with high intensity of cytosolic Dendra (yellow arrow) to better appreciate their very low number of puncta compared to those with low levels of cytosolic Dendra signal (white arrow). FIG. 12c, Flow cytometry analysis of direct Dendra fluorescence of HSC from 30 m old mice. Representative plot of n=4 mice. FIG. 12d, Representative images of full field (left) and higher magnification insets (right) of LSK cells from 30 m old mice incubated or not with leupeptin for 16 hours before immunostaining for Dendra. Exposure time is indicated in the insets. n=4 mice. FIG. 12e, CMA flux calculated as increase in puncta number after leupeptin treatment in HSC from young (4 m) mice and high and low Dendra intensity populations from old (30m) mice. n=9 fields from 4 mice per group. Average of cells per field and mean+s.e.m. are shown. FIG. 12f,g, Oxidized protein staining in 30 m old mice HSC populations FACS sorted according to their cytosolic Dendra fluorescence intensity. Representative images with higher magnification insets (FIG. 12f) and quantification of the intensity of oxidized proteins (FIG. 12g) are shown. n=26 (high intensity population) and 28 (low intensity population) fields from 4 mice. FIG. 12h,i, Cellular ROS levels (FIG. 12h) and GAPDH activity (i) in the same FACS sorted HSC populations as in FIG. 12f. Values of bars are mean+s.e.m. One-way ANOVA with Bonferroni post-hoc multiple comparison test (FIG. 12a, e) and unpaired t tests (FIG. 12g-i) were used. p<0.001 (***) and 0.0001 (****). ns: no statistical significance.

FIG. 13. Characterization of HSC with genetic or pharmacological activation of CMA. FIG. 13a,b, Immunostaining for human and mouse L2A in HSC from Ctrl and hL2A^{OE} mice. Quantification of the average fluorescence intensity per cell relative to Ctrl cells is shown in FIG. 13b. n=9 fields from 3 individual mice. FIG. 13c, Real-time quantitative PCR analysis of human L2A and mouse L2A in HSC from Ctrl and hL2A^{OE} mice expressed as fold mL2A mRNA levels in Ctrl cells. Mouse β-actin was used as housekeeping gene. n=3 independent experiments. FIG. 13d, Representative images of full field and higher magnification insets of HSC from 22-25 m old KFERQ-Dendra-hL2A^{OE} mice incubated or not with leupeptin for 16 hours before immunostaining for Dendra. FIG. 13e-g, Quantification of the CMA flux (as an increase in fluorescent puncta upon leupeptin treatment) in HSC from old Ctrl (from Extended FIG. 8d) and KFERQ-Dendra-hL2A^{OE} mice when averaging all cells independently of the intensity of their cytosolic Dendra staining (FIG. 13e) or when separately analyzing flux in cells with high and low cytosolic Dendra staining (FIG. 13f). The fraction of cells with high intensity of cytosolic Dendra staining in both groups of mice is shown in FIG. 13g. n=12 fields from 4 mice. FIG. 13h, Representative flow cytometry plots of HSC from young and old KFERQ-Dendra mice and old Dendra-hL2A^{OE} mice sorted for their cytosolic Dendra fluorescence intensity. FIGS. 13i-l, HSC frequency (i), LTC-IC frequency (j), levels of intracellular ROS (FIG. 13k) and PK activity (FIG. 13) in HSC from 7 m hL2A^{OE} mice with upregulated L2A expression since 4 m of age. n=3-6 mice/group. FIG. 13m, Representative direct fluorescence images (full field and higher magnification insets) of HSC from 22-25 months old KFERQ-Dendra mice ex vivo treated with vehicle or the CMA activator (10 μM CA) for 4 weeks (left). Quantification of fluorescent puncta per cell is shown on the right. n=9 fields from 3 mice (from 7-10 cells per field). FIG. 13n, Representative full field images and higher magnification insets of oxidized protein staining in HSC cells from 22-25 months old mice after 7 days of culture in presence of vehicle or CA (10 μM). Nuclei are stained with DAPI. Quantification is shown in FIG. 4m. Two-way ANOVA followed by Sidak's (FIG. 13b) or Tukey's (FIGS. 13c,d) multiple comparisons post-hoc test, unpaired t test (e.g., FIGS. 13i-m) and one-way ANOVA tests with Tukey's multiple comparison test (FIG. 13f), were used. P<0.0001 (****). ns: no statistical significance.

FIG. 14. The effect of CMA on polyunsaturated fatty acid metabolism in HSC is the basis for the role of CMA in HSC function. FIG. 14a, Polyunsaturated fatty acids that are generated by FADS2 activity decrease with age in human blood from healthy donor volunteers (n=250)62. Top metabolic changes positively and negatively correlating (Spearman's correlation) with aging were determined (left) and placement of 3 products of FADS2 activity in the graph is marked by the with circles as (1) for DGLA (Dihomo-γ-linolenic acid), (2) for AA (arachidonic acid) and (3) for DCA (Docosatetraenoic acid). Right shows levels of each of the three metabolites in 250 healthy individuals with age range 20-90 years. Pearson correlation was used for statistics. FIG. 14b, Scheme of the role of CMA in HSC function. Left: under basal conditions, functional CMA is required in HSC for protein quality control including that of enzymes involved in glucose metabolism. Failure of CMA leads to persistence of damaged enzymes and reduced glycolytic activity. Right: during HSC activation, CMA is required for increasing FADS2 activity the limiting enzyme in linoleic and a-linolenic metabolism, to activate this pathway and thus facilitate the metabolic switch from glucose to lipid metabolism. CMA changes the active/inactive enzyme ratio by selective removal of the inactive forms of FADS2. Acetylation of inactive forms of FADS2 during HSC activation completes a KFERQ-like motif in FADS2 that allows its recognition by hsc70 and subsequent targeting to lysosomes for degradation.

FIG. 15. Representative FACS data and gating strategy. Bead sorted CD34+ MNC from patients over 55 years of age were treated with compounds 77.1, 77.43, 77.36, B13, B19 or vehicle control for 72 hours in HSC maintenance cultures. The experimental readout is quantification of phenotypical HSC (HSPCs) by FACS analysis. Results show a detectable and significant increase in HSCs in an AML patient derived specimen treat with 77.1 and at least one other CMA activator compound. Results also identified a positive trend in HSC number from a HC derived specimen.

FIG. 16. CMA activators show differential effects of immature and HSPC populations ex vivo. The y-axis show the particular cell type as a percentage of the total single cells. FIG. 16a. Lineage negative cells. FIG. 16b. CD34+, CD38−, Lin− cells.

FIG. 17. Compounds CA77.1, CA77.43, and CA77.36 lead to significantly more long-term HSCs (LT-HSC) than vehicle upon short term ex vivo culture. FIG. 17a. CD90+, CD49f+, CD34+, CD38−, Lin-LT-HSC cells, FIG. 17b. CD90+, CD49f−, CD34+, CD38−, Lin− ST-HSC cells, FIG. 17c. CD90−, CD49f+, CD34+, CD38−, Lin-MPP.

FIG. 18. No detectable changes were observed in HSPC maintenance with short term culture. FIG. 18a. Lineage negative cells treated with vehicle or CMA modulators. FIG. 18b. CD34+, CD38−, Lin-HSPC cells treated with vehicle or CMA modulator compounds.

FIG. 19. No significant changes were observed in HSPC maintenance with short term culture of additional cell types. FIG. 19a. CD90+, CD49f+, CD34+, CD38−, Lin- LT-HSC cells treated with CMA modulators. FIG. 19b. CD90+, CD49f-, CD34+, CD38-, Lin- ST-HSC cells, FIG. 19c. CD90−, CD49f+, CD34+, CD38−, Lin-MPP cells.

FIG. 20. COH-1 Engraftment Check in healthy MNCs from a 23 year old female. Percent of the indicated cell type in MNCs treated with CMA activator compound 77.10 alone or with compound 77.10 and B13. FIG. 20a. hCD45 cells. FIG. 20b. T− cells. FIG. 20c. B-cells. FIG. 20d. Monocytes. FIG. 20e. Neutrophils. FIG. 20f. Erythrocytes. FIG. 20g. MK cells. FIG. 20h. CD34+CD38+cells. FIG. 20i. CD34+, CD38- cells. FIG. 20j. LT-HSCs. FIG. 20k. ST-HSCs. FIG. 201. MPPs. 77.10 and 77.1 are the same compound.

FIG. 21. COH-2 Engraftment Check in CC0471 AML MNCs from a 80 year old male. FIG. 21a. hCD45 cells. FIG. 21b. MPPs. FIG. 21c. CD34+CD38+cells. FIG. 21d. CD34+, CD38- cells. FIG. 21e. LT-HSCs. FIG. 21f. ST-HSCs. FIG. 21g. B Cells.

FIG. 21h. Erythrocytes. FIG. 21i. T cells. FIG. 21k. Monocytes. FIG. 21l. Neutrophils. FIG. 21m. MK Cells

### DETAILED DESCRIPTION OF THE INVENTION

The invention is based in part upon the discovery that chaperone-mediated autophagy (CMA), a selective form of lysosomal protein degradation, has a role in sustaining adult hematopoietic stem cell (HSC) function. Specifically, it was discovered that CMA is required for stem cell protein quality control and upregulation of fatty acid metabolism upon HSC activation. Further, it was demonstrated that CMA activity decreases with age in HSC and, importantly, genetic or pharmacological activation of CMA can restore functionality of old HSC.

The inventors discovered that functional CMA is required for HSC maintenance and function. In quiescent steady-state HSC, CMA contributes to quality control and the defense against oxidative stress by removing oxidized proteins. Whereas upon HSC activation, timely degradation by CMA of acetylation-tagged proteins is required to stimulate long chain fatty acid metabolism and accommodate the metabolic requirements of a dividing stem cell.

Other components of the proteostasis network have been reported to contribute to HSC homeostasis and function. Metabolic deregulation with massive accumulation of lipid droplets and increased glycolysis occurs upon macroautophagy blockage in neutrophil precursors. This metabolic phenotype is in clear contrast with the reduced glycolysis observed in CMA-deficient HSC.

Critically, loss of CMA in HSC cannot be compensated for by macroautophagy in these cells. Different from other cell types (i.e. fibroblasts, hepatocytes) that respond to CMA blockage with an upregulation of macroautophagy activity to preserve cellular proteostasis, HSC fall in the group of cells (also including retinal cells or activated T cells) in which macroautophagy activity remains unaltered upon CMA blockage.

This lack of compensatory upregulation is a determining factor for the early in life loss of proteostasis observed in HSC when CMA is non-functional. Differences in timing, signaling pathways and substrate recognition may be responsible for the inability of these proteolytic systems to compensate for each other in HSC. The sustained lower quality control of CMA-deficient HSC through life has likely a cumulative effect at the level of proteotoxicity, evidenced by the worsening of the L2AKO HSC phenotype with age.

The consequences of CMA failure in HSC go beyond the changes in lipid metabolism. Comparative proteomic analysis demonstrate that CMA-deficient cells are unable to carry out the overall proteome remodeling required during the process of activation. Failure to timely modulate levels of proteins involved in processes such as cell cycle, cytoskeletal organization, or in the regulation of other proteostasis components, to the functional impairment of HSC. The selectivity and timing of CMA degradation is likely behind the cell type-specific functions described for this type of autophagy. For example, CMA facilitates T cell activation by directly degrading proteins that actively repress T cell activation, whereas here we uncovered that CMA partakes in regulation of the metabolic state of HSC upon activation. Interestingly, the mechanisms and metabolic pathways regulated by CMA appear also to be cell type and context specific as, for example, CMA participates in maintenance of pluripotency of embryonic stem cells also thorough metabolic changes but in that case by limiting a-ketoglutarate production, which ultimately affects activity of DNA demethylases involved in pluripotency.

Precluding the age-dependent decline of CMA in HSC is effective in preserving stem cell function and thus preventing the expansion of a functionally compromised HSC pool that observed in aging. Surprisingly, the upregulation of CMA even later in life at a time when HSC function is already compromised, is efficient in restoring HSC function. These findings support that reactivation of CMA in HSC is possible and still has a beneficial effect at advanced age. The deficiencies in proteostasis maintenance and self-renewal capacity of HSC with defective CMA and the observed improvements in these two functions in old HSC upon genetic or pharmacological upregulation of CMA both in mouse and human HSC, unveils CMA as a target to restore hematopoietic stem cell function in aging.

Together, these findings provide mechanistic insights into a new role for CMA in sustaining quality control, appropriate energetics, and overall long-term hematopoietic stem cell function. As such, CMA is a promising therapeutic target to enhance hematopoietic stem cell function in conditions such as aging, certain disease states, and stem cell transplantation.

Activation of CMA preserves the “stem cell-ness” of HSCs allowing for their expansion. Stem cells tend to spontaneously differentiate during proliferation, and it is difficult to culture stem cells while maintaining the undifferentiated state of the cells.

The discovery of the ability to expand HSC extensively while retaining the HSC cell phenotype has significant therapeutic implications.

The present disclosure provides for methods for expanding HSCs while maintaining them in an undifferentiated state by culturing them in the presence of a CMA activator. In certain embodiments, the CMA activators described herein are added to a cell culture medium comprising the HSCs. The stem cells are cultured in any media known in the art. Also provide by the disclosure are methods of suppressing the differentiation of HSCs by contacting the population of HSCs with a CMA activator. The method is performed in vitro or in vivo.

The population of stem cells are cultured for a period of time until a sufficient number of HSCs are produced. The starting cell density is about 5,000 to 50,000 cells/cm². For example, the starting cell density is 20,000 cells/cm². The period of time is 1, 2, 3, 4, 5, 6, 7 or more days.

The methods provide a substantially pure population of HSCs. A substantially pure population of HSCs is a population of HSCs where at least 70%, 75%, 80%, 85%, 90%, 95% or more for the cells express a cell surface marker indicative of an HSC on their cell surface. HSC cell surface markers include for example Lin(CD2, CD3, CD4, CD8, CD14, CD15, CD19, CD20, CD56, GLYA, CD71, CD33)− CD34+CD38− CD123-CD45RA− (CD90+ or CD49f+) IL1RAP− Tim-3. Cell surface expression of HSC cell surface markers can be determined by methods know in the art, such as for example, FACS.

The resultant HSC population can be further purified and/or expanded by the methods know in the art and described above.

The present disclosure also provides methods for expanding a population of blood cells while by culturing them in the presence of CMA activator.

In certain embodiments, the CMA activators described herein are added to a cell culture medium comprising the blood cells. The blood cells are cultured in any media known in the art.

The population of blood are cultured for a period of time until a sufficient number of blood cells are produced. The starting cell density is about 5,000 to 50,000 cells/cm². For example, the starting cell density is 20,000 cells/cm². The period of time is 1, 2, 3, 4, 5, 6, 7 or more days.

The methods provide a substantially pure population of blood. A substantially pure population of blood cells is a population of HSCs where at least 70%, 75%, 80%, 85%, 90%, 95% or more for the cells express a cell surface marker indicative of the particular subtype of blood cells. Cell surface expression of blood cell surface markers can be determined by methods know in the art, such as for example, FACS.

The resultant blood cell population can be further purified and/or expanded by the methods know in the art and described above.

The blood cell is a red blood cell or a white blood cell. The white blood cell is a leukocyte such as a lymphocyte. The lymphocyte can be a B-cell or a T-cell. The T-cell can be a CAR-T cell.

The HSCs, the blood cells, and the pharmaceutical compositions comprising the cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “THE SCIENCE AND PRACTICE OF PHARMACY”, 23rdth edition, 2020, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about using agents delaying absorption, for example, alum inurn monostearate and gelatin. According to the presently disclosed subject matter, however, any vehicle, diluent, or additive used would have to be compatible with the presently disclosed stem-cell-derived precursors.

Viscosity of the compositions can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel, or another liquid form, such as a time release form or liquid-filled form).

Those skilled in the art will recognize that the components of the compositions should be selected to be chemically inert and will not affect the viability or efficacy of the HSCs. This will present no problem to those skilled in chemical and pharmaceutical principles, or problems can be readily avoided by reference to standard texts or by simple experiments (not involving undue experimentation), from this disclosure and the documents cited herein.

In certain non-limiting embodiments, the HSCs described herein are comprised in a composition that further contains growth factors for promoting maturation of the cells.

The HSCs produced by the disclosed methods and pharmaceutical compositions thereof can be used for treating a variety of hematological and extra-hematological diseases, such as cancer as described herein.

The HSCs and the CMA activator can be administered or provided directly to a subject for treating or preventing hematological and extra-hematological disease. Hematologic diseases are disorders which primarily affect the blood & blood-forming organs. Hematologic diseases include rare genetic disorders, cancer, bone marrow failure, bleeding disorders, and complications from chemotherapy or transfusions.

Further included by the disclosure are methods by administering an effective amount of CMA activator to a subject suffering a hematological and extra-hematological disease. The disease or disorder can be a result of a deficiency or a dysfunction of HSCs. The method treats, prevents, or alleviates a sign or symptom of the disease or disorder.

For example, the methods treat cancers and other disorders of the blood and immune systems.

Diseases and disorders to be treated or prevented by a method of the invention include, for example, the following: Red blood cell disorders, Anemia, Congenital dyserythropoietic anemia, Congenital sideroblastic anemia, G6PD deficiency, Hemochromatosis, Hemolytic anemia, Hemolytic disease of the newborn, Hydrops fetalis, Iron deficiency anemia, Iron-refractory iron deficiency anemia (IRIDA), Megaloblastic anemia (including pernicious anemia), Pyruvate kinase (PK) deficiency, Sickle cell disease, Spherocytosis, Thalassemia, White blood cell disorders, Cyclic neutropenia, Severe congenital neutropenia (Kostmann syndrome), Chronic granulomatous disease, Leukocyte adhesion deficiency, Myeloperoxidase deficiency, Bone marrow failure syndromes, Aplastic anemia, Congenital amegakaryocytic thrombocytopenia, Diamond-Blackfan anemia, Dyskeratosis congenita, Fanconi anemia, Myelodysplastic syndrome (MDS), Pearson syndrome, Shwachman-Diamond syndrome, Thrombocytopenia absent radius, Bleeding disorders, Hemophilia, Hypofibrinogenemia and dysfibrinogenemia, Platelet function disorders, Thrombocytopenia, von Willebrand disease, Thrombosis and anticoagulation disorders, Antithrombin deficiency, Factor V Leiden, Protein C deficiency, Protein S deficiency, Prothrombin gene mutation, Stroke, Thrombosis, Autoimmune blood cell disorders, Autoimmune hemolytic anemia, Evans syndrome, Immune thrombocytopenia (ITP), Myeloproliferative neoplasms (MPNs), and Polycythemia.

Disorders to be treated and/or prevented by a method of this disclosure include myeloproliferative disorders. (MPN), previously called myeloproliferative disorders, are a group of disorders characterized by the overproduction of one or more blood cell type (white blood cells, red blood cells, and/or platelets). At diagnosis, most myeloproliferative neoplasms are benign but over time may evolve into a malignant (cancerous) disease. MPNs include for example Polycythemia vera (PV), Essential Thrombocythemia (ET), Myelofibrosis such as Primary Myelofibrosis (PMF), Juvenile Myleomonocytic Leukemia (JMML), Chronic Neutrophilic Leukemia, Chronic Eosinophilic Leukemia/Hypereosinophilic Syndromes (HES), Mast Cell Disease, Chronic myelogenous leukemia, and Acute myeloid leukemia (AML).

An effective amount of a CMA Activator is an amount sufficient to inhibit the progression of a disease or disorder, cause a regression of a disease or disorder, reduce symptoms of a disease or disorder, or significantly alter a level of a marker of a disease or disorder, or reduce the probability of a treated subject of developing the disease or disorder relative to an untreated subject of the same species. CMA Activators

CMA activators have been disclosed previously. CMA activators useful in the methods of this disclosure include compounds disclosed in U.S. application Ser. Nos. 17/283,613 and 17/270522, and, U.S. Pat. Nos. 9,512,092, 9,890,143, 10,766,886, and 10,189,827, each of which is hereby incorporated by reference for its disclosure of CMA modulating and CMA activatingcompounds.

The CMA Activating compound may be a compound of Formula I

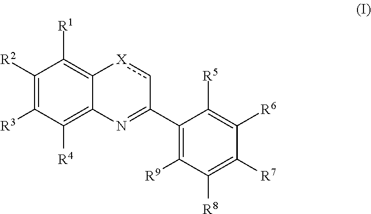

or a pharmaceutically acceptable salt thereof. Within Formula I the variables carry the following definitions.

X is O and [symbol] is a single bond, or X is N and [symbol] is an aromatic bond.

R¹, R³, and R⁴ are independently chosen from hydrogen, halogen, hydroxyl, C₁-C6alkyl, and C₁-C6alkoxy.

R¹ is halogen.

R⁵, R⁶, R⁸, and R⁹ are independently chosen from hydrogen, halogen, hydroxyl, C₁-C₆alkyl, and C₁-C₆alkoxy.

R⁷ is —NR¹⁰COR¹¹ or —NR¹⁰SO₂R¹¹, or R⁷ is phenyl, naphthyl, and mono- or bi-cyclic heteroaryl each of which is optionally substituted with halogen, hydroxyl, cyano, —CHO, —COOH, amino, and C₁-C₆alkyl in which any carbon-carbon single bond is optionally replaced by a carbon-carbon double or triple bond, any methylene group is optionally replaced by O, S, or NR¹², and optionally substituted with one or more substituents independently chosen from halogen, hydroxyl, cyano, amino, and oxo; and one substituent chosen from —NR¹⁰COR¹¹ and NR¹⁰SO₂R¹¹.

R¹⁰ is independently chosen at each occurrence from hydrogen and C₁-C₆alkyl.

R¹¹ is independently chosen at each occurrence from hydrogen, C₁-C₆alkyl, C₁-C₂haloalkyl, monocyclic aryl and heteroaryl, each of which monocyclic aryl and heteroaryl is optionally substituted with one or more substituents independently chosen from halogen, hydroxyl, cyano, C₁-C₆alkyl, C₁-C₆alkoxy, C₁-C₂haloalkyl, and C₁-C₂haloalkoxy/

R¹² is hydrogen, C₁-C₆alkyl, or (C₁₃-C₇cycloalkyl)C₀-C₂alkyl.

The CMA Activating compound may be a compound of Formula II

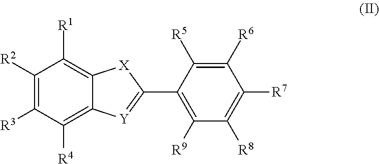

or a pharmaceutically acceptable salt thereof. Within Formula II the variables carry the following definitions.

X is O, C(R¹⁰R¹¹), C═O, N(R¹²), S, or S=O and Y is CR¹⁰ or N.

R¹R², R³, and R⁴ are independently chosen from hydrogen, halogen, hydroxyl, C₁-C₆alkyl, and C₁-C₆alkoxy.

R⁵, R⁶, R⁸, and R⁹ are independently chosen from hydrogen, halogen, hydroxyl, C₁-C₆alkyl, and C₁-C₆alkoxy.

R 7 is —NR²⁰COR²¹ or —NR²⁰SO₂R²¹, or R⁷ is a phenyl, naphthyl, pyridyl, pyrimidinyl, pyrazinyl, thienyl, thiazolyl, imidazolyl, oxazolyl, triazolyl, quinolinyl, or isoquinolinyl group; each of which is optionally substituted with one or more substituents independently chosen from halogen, hydroxyl, cyano, —CHO, —COOH, amino, and C₁-C₆alkyl in which any carbon-carbon single bond is optionally replaced by a carbon-carbon double or triple bond, any methylene group is optionally replaced by O, S, or NR²², and optionally substituted with one or more substituents independently chosen from halogen, hydroxyl, cyano, amino, and oxo; and each of which is optionally substituted with one substituent chosen from —N(R²⁰)COR²¹ and —N(R²⁰)SO₂R²¹.

R¹⁰ and R¹¹ are independently chosen from hydrogen, halogen, hydroxyl, amino, cyano, C₁-C₆alkyl, C₁-C₆alkoxy, (C₃-C₆cycloalkyl)C₀-C₂alkyl, C₁-C₂haloalkyl, and C₁-C₂haloalkoxy.

R¹² is hydrogen, C₁-C₆alkyl, or (C₃-C₆cycloalkyl)C₀-C₂alkyl.

R²⁰ is hydrogen or C₁-C₆alkyl.

R²¹ is independently chosen at each occurrence from hydrogen, C₁-C₆alkyl, C₁-C₂haloalkyl, monocyclic aryl and heteroaryl, each of which monocyclic aryl and heteroaryl is optionally substituted with one or more substituents independently chosen from halogen, hydroxyl, cyano, C₁-C₆alkyl, C₁-C₆alkoxy, C₁-C₂haloalkyl, and C₁-C₆haloalkoxy/

R²² is hydrogen, C₁-C₆alkyl, or C₃-C₇cycloalkyl)C₀-C₂alkyl; with the proviso that the compound is not N-(4-(6-chlorobenzo[d]oxazol-2-yl)phenypisobutyramide.

The CMA Activating compound may be a compound of Formula III-A and Formula III-B

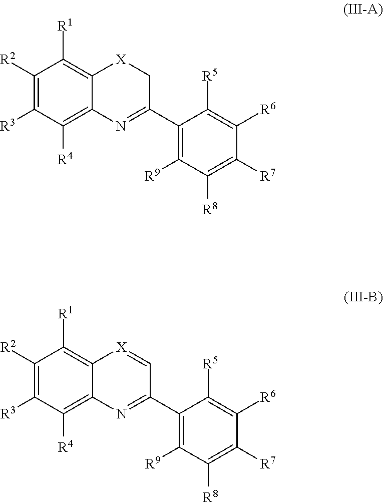

or a pharmaceutically acceptable salt thereof. The variables in Formula III carry the following definitions.

R¹, R², R³, R⁴, R⁵, R⁶, and R⁹ of formula (III) are independently H, hydroxyl, halogen, SH, NO², CFR³, COOH, COOR¹⁰, CHO, CN, NHR₂, NHRR¹⁰, NHCONH₂, NHCONHR¹⁰, NHCOR¹⁰, NHSO₂R¹⁰, OCR¹⁰, COR¹⁰, CH₂R¹⁰, CON(R¹⁰R¹¹), CH═N—OR¹⁰, CH═NR¹⁰, OR¹⁰, SR¹⁰, SOR¹⁰, SO₂R¹⁰, COOR¹⁰, CH₂N(R¹⁰R¹¹), N(R¹⁰R¹¹), or optionally substituted lower alkyl, alkenyl, alkynyl, cycloalkyl, heterocycloalkyl, aryl, heteroaryl, aralkyl, or heteroaralkyl; wherein the optional substituent is one or more of F, Cl, Br, I, OH, SH, NO₂, COOH, COOR¹⁰, R¹⁰, CHO, CN, NH₂, NHR¹⁰, NHCONH₂, NHCONHR¹⁰, NHCOR¹⁰, NHSO₂R¹⁰, HOCR¹⁰, COR¹⁰, CH₂R¹⁰, CON(R¹⁰R¹¹), CH═N—OR¹⁰, CH═NR¹⁰, OR¹⁰, SR¹⁰, SOR¹⁰, SO₂R¹⁰, COOR¹⁰, SCH₂N(R¹⁰R¹¹), N(R¹⁰R¹¹);

R⁷ of formula (II) is H, hydroxyl, halogen, CF₃, CN, OCF₃, COOH, COOCH₃, COOR¹⁰, COO(CH₂)₂Si(CH₃)₃, COOR¹⁰Si(CH₃)₃, NHCOCH₃, C═C—CH₂OH, C═C—R¹⁰—OH or optionally substituted alkyl, aryl, heteroaryl, aralkyl, heteroarylalkyl, cyclic or heterocyclic; wherein the optional substituent is one or more of F, Cl, Br, I, OH, SH, NO₂, CH₃, R¹⁰, COOH, COOR¹⁰, CHO, CN, NH₂, NHR¹⁰, NHCONH₂, NHCONHR ¹⁰, NHCOR¹⁰, NHSO₂R ¹⁰, HOCR¹⁰, COR¹⁰, CH₂R¹⁰, CON(R¹⁰R¹¹), CH═N—OR¹⁰, CH═NR¹⁰, OR¹⁰, SR¹⁰, SOR¹⁰, SO₂R¹⁰, COOR¹⁰, CH₂N(R¹⁰R¹¹), N(R¹⁰R¹¹

R¹⁰ and R¹¹ are independently H or C¹-C⁶ alkyl; and X is CH₂, C═O, N, O, S or S═O for Formula III-A, and X is CH or N for Formula III-B; and Y is N or CH. In certain embodiments the CMA activating compound is a compound of Formula III-A, X is O and Y is N.

CMA Activators of Formula I include:

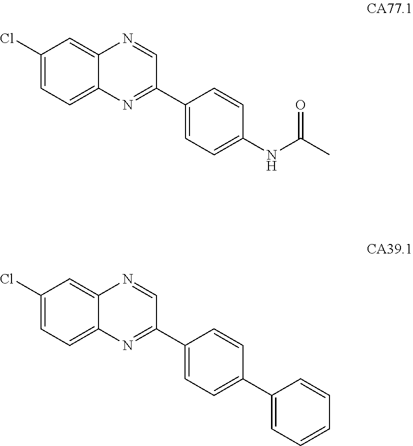 [SMILES: CC(=O)Nc1ccc(-c2cnc3cc(Cl)ccc3n2)cc1 ; Clc1ccc2nc(-c3ccc(-c4ccccc4)cc3)cnc2c1]

The disclosure provides CMA Activators of Formula I, in which the activator is a compound or salt of Table 1.

TABLE 1
Benzoxazines and Quinazolines

| Cpd. # | Chemical Structure | Name |
|---|---|---|
| 1 (CA-3-55) | 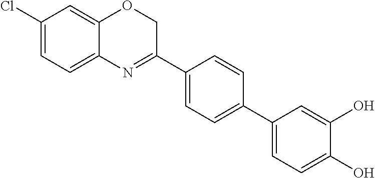 [SMILES: Oc1ccc(-c2ccc(C3=Nc4ccc(Cl)cc4OC3)cc2)cc1O] | 4′-(7-chloro-2H- benzo[b][1,4]oxazin-3-yl)- [1,1-biphenyl]-3,4-diol |
| 2 (CA-3-67) | 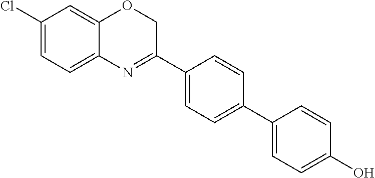 [SMILES: Oc1ccc(-c2ccc(C3=Nc4ccc(Cl)cc4OC3)cc2)cc1] | 4′-(7-chloro-2H- benzo[b][1,4]oxazin-3-yl)- [1,1′-biphenyl]-4-ol |
| 3 (CA-3-69) | 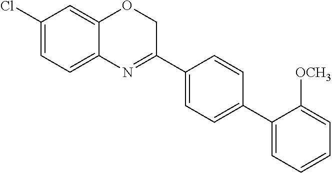 [SMILES: COc1ccccc1-c1ccc(C2=Nc3ccc(Cl)cc3OC2)cc1] | 7-chloro-3-(2′-methoxy-[1,1′- biphenyl]-4-yl)-2H- benzo[b][1,4]oxazine |
| 4 (CA-3-70) | 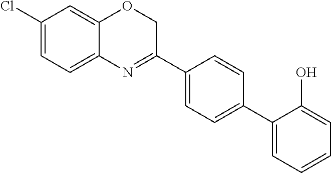 [SMILES: Oc1ccccc1-c1ccc(C2=Nc3ccc(Cl)cc3OC2)cc1] | 7-chloro-3-(2′-hydroxy-[1,1′- biphenyl]-4-yl)-2H- benzo[b][1,4]oxazine |
| 5 (CA39.1) | 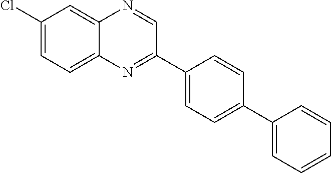 [SMILES: Clc1ccc2nc(-c3ccc(-c4ccccc4)cc3)cnc2c1] | 2-([1,1′-biphenyl]-4-yl)-6- chloroquinoxaline |
| 6 (CA77.1) | 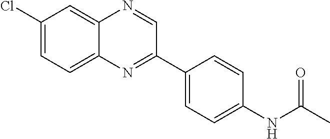 [SMILES: CC(=O)Nc1ccc(-c2cnc3cc(Cl)ccc3n2)cc1] | N-(4-(6-chloroquinoxalin-2- yl)phenyl)acetamide |
| 7 (CA77.11) | 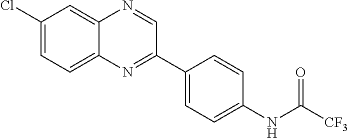 [SMILES: O=C(Nc1ccc(-c2cnc3cc(Cl)ccc3n2)cc1)C(F)(F)F] | N-(4-(6-chloroquinoxalin-2- yl)phenyl)-2,2,2- trifluoroacetamide |
| 8 (CA77.12) | 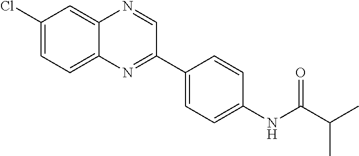 [SMILES: CC(C)C(=O)Nc1ccc(-c2cnc3cc(Cl)ccc3n2)cc1] | N-(4-(6-chloroquinoxalin-2- yl)phenyl)isobutyramide |
| 9 (CA77.13) | 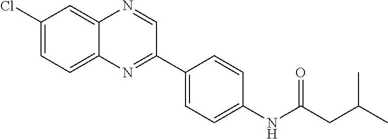 [SMILES: CC(C)CC(=O)Nc1ccc(-c2cnc3cc(Cl)ccc3n2)cc1] | N-(4-(6-chloroquinoxalin-2- yl)phenyl)butyramide |
| 10 (CA77.14) | 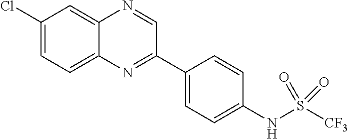 [SMILES: O=S(=O)(Nc1ccc(-c2cnc3cc(Cl)ccc3n2)cc1)C(F)(F)F] | N-(4-(6-chloroquinoxalin-2- yl)phenyl)-1,1,1- trifluoromethanesulfonamide |
| 11 (CA77.15) | 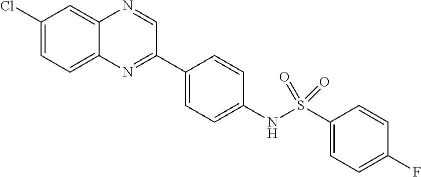 [SMILES: O=S(=O)(Nc1ccc(-c2cnc3cc(Cl)ccc3n2)cc1)c1ccc(F)cc1] | N-(4-(6-chloroquinoxalin-2- yl)phenyl)-4- fluorobenzenesulfonamide |
| 12 (CA77.16) | 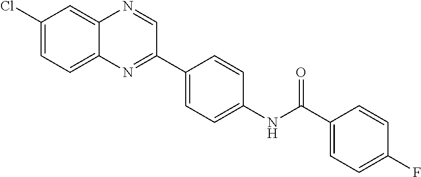 [SMILES: O=C(Nc1ccc(-c2cnc3cc(Cl)ccc3n2)cc1)c1ccc(F)cc1] | N-(4-(6-chloroquinoxalin-2- yl)phenyl)-4- fluorobenzamide |
| 13 (CA39.12) | 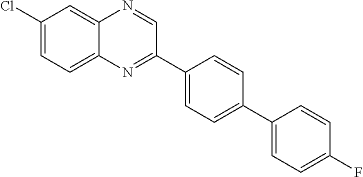 [SMILES: Fc1ccc(-c2ccc(-c3cnc4cc(Cl)ccc4n3)cc2)cc1] | 6-chloro-2-(4′-fluoro-[1,1′- biphenyl]-4-yl)quinoxaline |
| 14 (CA39.13) | 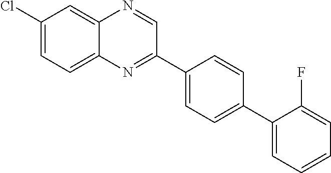 [SMILES: Fc1ccccc1-c1ccc(-c2cnc3cc(Cl)ccc3n2)cc1] | 6-chloro-2-(2′-fluoro-[1,1′- biphenyl]-4-yl)quinoxaline |
| 15 (CA39.14) | 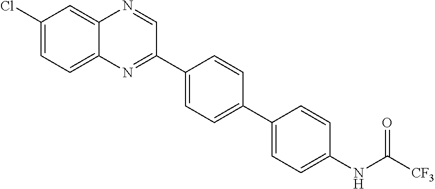 [SMILES: O=C(Nc1ccc(-c2ccc(-c3cnc4cc(Cl)ccc4n3)cc2)cc1)C(F)(F)F] | N-(4′-(6-chloroquinoxalin-2- yl)-[1,1′-biphenyl]-4-yl)- 2,2,2-trifluoroacetamide |
| 16 (CA39.15) | 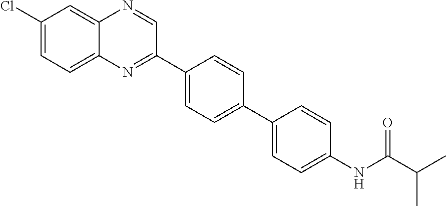 [SMILES: CC(C)C(=O)Nc1ccc(-c2ccc(-c3cnc4cc(Cl)ccc4n3)cc2)cc1] | N-(4′-(6-chloroquinoxalin-2- yl)-[1,1′-biphenyl]-4- yl)isobutyramide |
| 17 (CA39.16) | 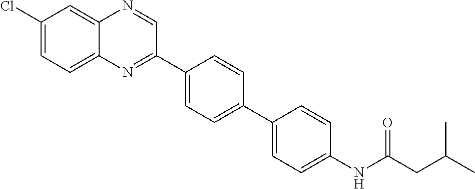 [SMILES: CC(C)CC(=O)Nc1ccc(-c2ccc(-c3cnc4cc(Cl)ccc4n3)cc2)cc1] | N-(4′-(6-chloroquinoxalin-2- yl)-[1,1′-biphenyl]-4- yl)butyramide |
| 18 (CA39.17) | 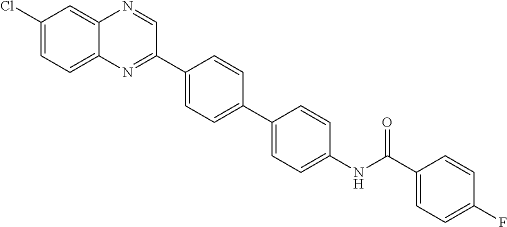 [SMILES: O=C(Nc1ccc(-c2ccc(-c3cnc4cc(Cl)ccc4n3)cc2)cc1)c1ccc(F)cc1] | N-(4′-(6-chloroquinoxalin-2- yl)-[1,l′-biphenyl]-4-yl)-4- fluorobenzamide |
| 19 (CA39.18) | 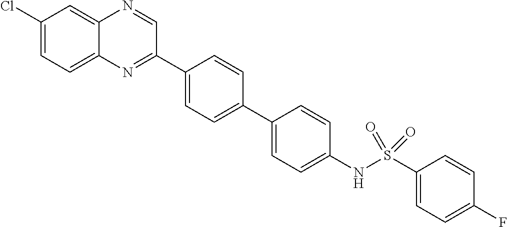 [SMILES: O=S(=O)(Nc1ccc(-c2ccc(-c3cnc4cc(Cl)ccc4n3)cc2)cc1)c1ccc(F)cc1] | N-(4′-(6-chloroquinoxalin-2- yl)-[1,1′-biphenyl]-4-yl)-4- fluorobenzenesulfonamide |
| 20 CA39.19 | 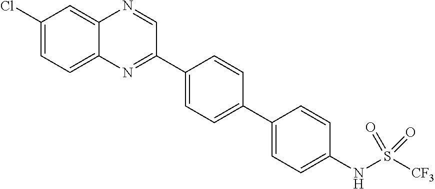 [SMILES: O=S(=O)(Nc1ccc(-c2ccc(-c3cnc4cc(Cl)ccc4n3)cc2)cc1)C(F)(F)F] | N-(4′-(6-chloroquinoxalin-2- yl)-[1,1′-biphenyl]-4-yl)- 1,1,1- trifluoromethanesulfonamide |
| 21 77.32 | 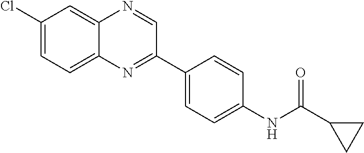 [SMILES: O=C(Nc1ccc(-c2cnc3cc(Cl)ccc3n2)cc1)C1CC1] | N-(4-(6-chloroquinoxalin-2- yl)phenyl)cyclopropane- carboxamide |
| 22 77.33 | 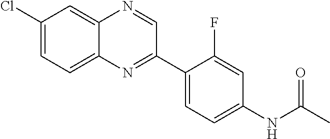 [SMILES: CC(=O)Nc1ccc(-c2cnc3cc(Cl)ccc3n2)c(F)c1] | N-(4-(6-chloroquinoxalin-2- yl)-3-fluorophenyl)acetamide |
| 23 77.34 | 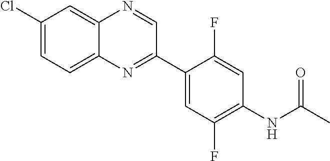 [SMILES: CC(=O)Nc1cc(F)c(-c2cnc3cc(Cl)ccc3n2)cc1F] | N-(4-(6-chloroquinoxalin-2- yl)-2,5-difluorophenyl) acetamide |
| 24 77.35 | 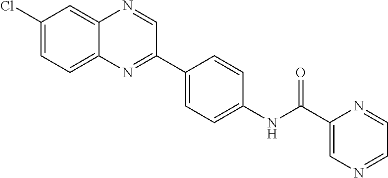 [SMILES: O=C(Nc1ccc(-c2cnc3cc(Cl)ccc3n2)cc1)c1cnccn1] | N-(4-(6-chloroquinoxalin-2- yl)phenyl)pyrazine-2- carboxamide |
| 25 77.36 | 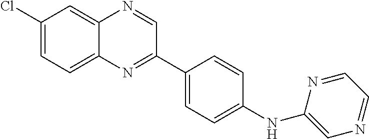 [SMILES: Clc1ccc2nc(-c3ccc(Nc4cnccn4)cc3)cnc2c1] | N-(4-(6-chloroquinoxalin-2- yl)phenyl)pyrazin-2-amine |
| 26 77.43 | 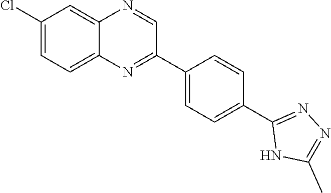 [SMILES: Cc1nnc(-c2ccc(-c3cnc4cc(Cl)ccc4n3)cc2)[nH]1] | 6-chloro-2-(4-(5-methyl-4H- 1,2,4-triazol-3- yl)phenyl)quinoxaline |
| 27 77.45 | 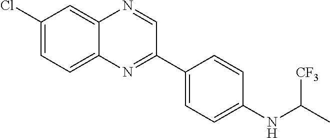 [SMILES: CC(Nc1ccc(-c2cnc3cc(Cl)ccc3n2)cc1)C(F)(F)F] | 4-(6-chloroquinoxalin-2-yl)- N-(1,1,1-trifluoropropan-2- yl)aniline |
| 28 39.25 | 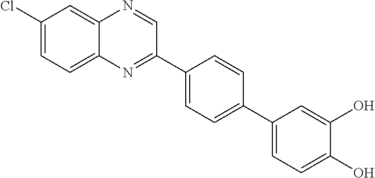 [SMILES: Oc1ccc(-c2ccc(-c3cnc4cc(Cl)ccc4n3)cc2)cc1O] | 4′-(6-chloroquinoxalin-2-yl)- [1,1′-biphenyl]-3,4-diol |
| 29 39.28 | 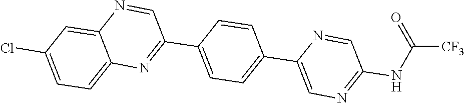 [SMILES: O=C(Nc1cnc(-c2ccc(-c3cnc4cc(Cl)ccc4n3)cc2)cn1)C(F)(F)F] | N-(5-(4-(6-chloroquinoxalin- 2-yl)phenyl)pyrazin-2-yl)- 2,2,2-trifluoroacetamide |
| 30 67-10 | 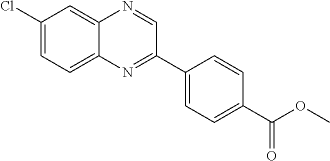 [SMILES: COC(=O)c1ccc(-c2cnc3cc(Cl)ccc3n2)cc1] | methyl 4-(6- chloroquinoxalin-2- yl)benzoate |
| 31 67-11 | 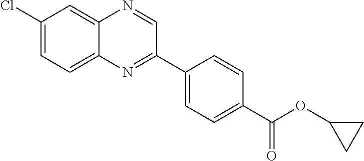 [SMILES: O=C(OC1CC1)c1ccc(-c2cnc3cc(Cl)ccc3n2)cc1] | cyclopropyl 4-(6- chloroquinoxalin-2- yl)benzoate |
| 32 67-12 | 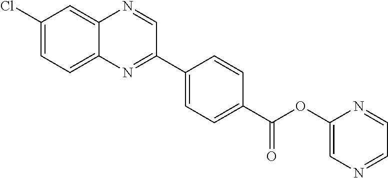 [SMILES: O=C(Oc1cnccn1)c1ccc(-c2cnc3cc(Cl)ccc3n2)cc1] | pyrazin-2-yl 4-(6- chloroquinoxalin-2- yl)benzoate |
| 33 55.1 | 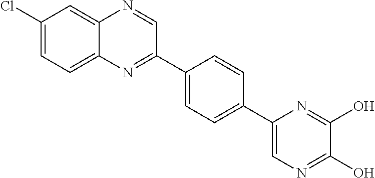 [SMILES: Oc1ncc(-c2ccc(-c3cnc4cc(Cl)ccc4n3)cc2)nc1O] | 5-(4-(6-chloroquinoxalin-2- yl)phenyl)pyrazine-2,3-diol |
| 34 55.21 | 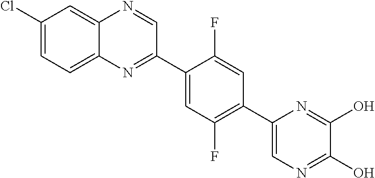 [SMILES: Oc1ncc(-c2cc(F)c(-c3cnc4cc(Cl)ccc4n3)cc2F)nc1O] | 5-(4-(6-chloroquinoxalin-2- yl)-2,5-difluorophenyl) pyrazine-2,3-diol |

The CMA Activator can be a compound of Formula II as shown in Table 2 or a salt thereof.

TABLE 2
Benzoxazoles

| Cmp. # | Chemical Structure | Name |
|---|---|---|
| 35 77.20 | 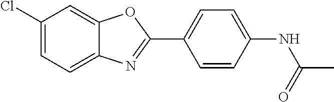 [SMILES: CC(=O)Nc1ccc(-c2nc3ccc(Cl)cc3o2)cc1] | N-(4-(6-chlorobenzo[d]oxazol-2- yl)phenyl)acetamide |
| 36 77.21 | 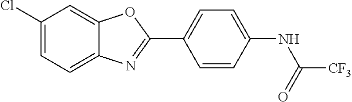 [SMILES: O=C(Nc1ccc(-c2nc3ccc(Cl)cc3o2)cc1)C(F)(F)F] | N-(4-(6-chlorobenzo[d]oxazol-2- yl)phenyl)-2,2,2-trifluoroacetamide |
| 37 77.21-5 | 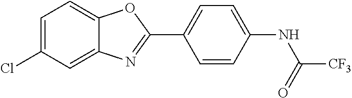 [SMILES: O=C(Nc1ccc(-c2nc3cc(Cl)ccc3o2)cc1)C(F)(F)F] | N-(4-(5-chlorobenzo[d]oxazol-2- yl)phenyl)-2,2,2-trifluoroacetamide |
| 38 77.23 | 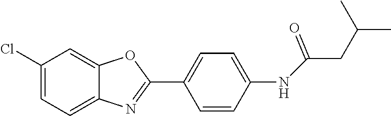 [SMILES: CC(C)CC(=O)Nc1ccc(-c2nc3ccc(Cl)cc3o2)cc1] | N-(4-(6-chlorobenzo[d]oxazol-2- yl)phenyl)-3-methylbutanamide |
| 39 77.24 | 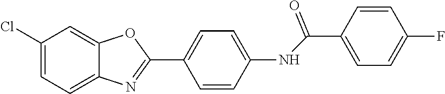 [SMILES: O=C(Nc1ccc(-c2nc3ccc(Cl)cc3o2)cc1)c1ccc(F)cc1] | N-(4-(6-chlorobenzo[d]oxazol-2- yl)phenyl)-4-fluorobenzamide |
| 40 77.25 | 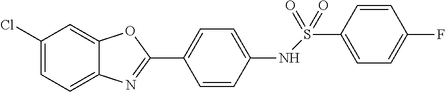 [SMILES: O=S(=O)(Nc1ccc(-c2nc3ccc(Cl)cc3o2)cc1)c1ccc(F)cc1] | N-(4-(6-chlorobenzo[d]oxazol-2- yl)phenyl)-4- fluorobenzenesulfonamide |
| 41 77.26 | 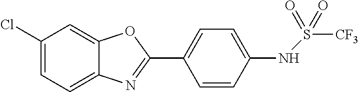 [SMILES: O=S(=O)(Nc1ccc(-c2nc3ccc(Cl)cc3o2)cc1)C(F)(F)F] | N-(4-(6-chlorobenzo[d]oxazol-2- yl)phenyl)-1,1,1- trifluoromethanesulfonamide |
| 42 77.27 | 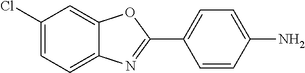 [SMILES: Nc1ccc(-c2nc3ccc(Cl)cc3o2)cc1] | 4-(6-chlorobenzo[d]oxazol-2- yl)aniline |
| 43 (77.28) | 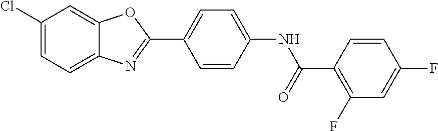 [SMILES: O=C(Nc1ccc(-c2nc3ccc(Cl)cc3o2)cc1)c1ccc(F)cc1F] | N-(4-(6-chlorobenzo[d]oxazol-2- yl)phenyl)-2,4-difluorobenzamide |
| 44 77.29 | 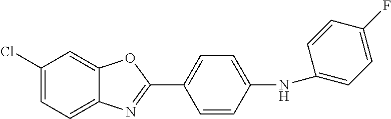 [SMILES: Fc1ccc(Nc2ccc(-c3nc4ccc(Cl)cc4o3)cc2)cc1] | 4-(6-chlorobenzo[d]oxazol-2-yl)-N- (4-fluorophenyl)aniline |
| 45 77.30 | 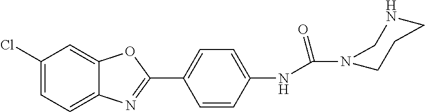 [SMILES: O=C(Nc1ccc(-c2nc3ccc(Cl)cc3o2)cc1)N1CCCNC1] | N-(4-(6-chlorobenzo[d]oxazol-2- yl)phenyl)tetrahydropyrimidine- 1(2H)-carboxamide |
| 46 77.31 | 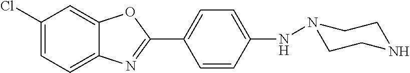 [SMILES: Clc1ccc2nc(-c3ccc(NN4CCNCC4)cc3)oc2c1] | N-(4-(6-chlorobenzo[d]oxazol-2- yl)phenyl)piperazin-1-amine |
| 47 77.37 | 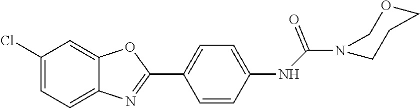 [SMILES: O=C(Nc1ccc(-c2nc3ccc(Cl)cc3o2)cc1)N1CCCOC1] | N-(4-(6-chlorobenzo[d]oxazol-2- yl)phenyl)-1,3-oxazinane-3- carboxamide |
| 48 77.38 | 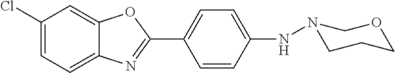 [SMILES: Clc1ccc2nc(-c3ccc(NN4CCCOC4)cc3)oc2c1] | N-(4-(6-chlorobenzo[d]oxazol-2- yl)phenyl)-1,3-oxazinan-3-amine |
| 49 77.39 | 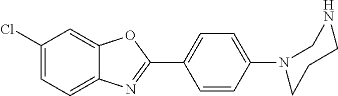 [SMILES: Clc1ccc2nc(-c3ccc(N4CCCNC4)cc3)oc2c1] | 6-chloro-2-(4-(tetrahydropyrimidin- 1(2H)-yl)phenyl)benzo[d]oxazole |
| 50 77.40 | 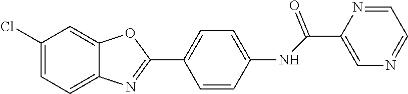 [SMILES: O=C(Nc1ccc(-c2nc3ccc(Cl)cc3o2)cc1)c1cnccn1] | N-(4-(6-chlorobenzo[d]oxazol-2- yl)phenyl)pyrazine-2-carboxamide |
| 51 77.41 | 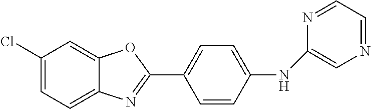 [SMILES: Clc1ccc2nc(-c3ccc(Nc4cnccn4)cc3)oc2c1] | N-(4-(6-chlorobenzo[d]oxazol-2- yl)phenyl)pyrazin-2-amine |
| 51 39.26 | 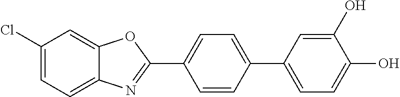 [SMILES: Oc1ccc(-c2ccc(-c3nc4ccc(Cl)cc4o3)cc2)cc1O] | 4′-(6-chlorobenzo[d]oxazol-2-yl)- [1,1′-biphenyl]-3,4-diol |
| 52 77.40 | 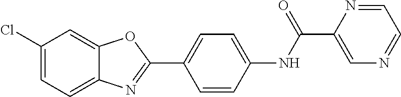 [SMILES: O=C(Nc1ccc(-c2nc3ccc(Cl)cc3o2)cc1)c1cnccn1] | N-(4-(6-chlorobenzo[d]oxazol-2- yl)phenyl)pyrazine-2-carboxamide |
| 53 67.2 | 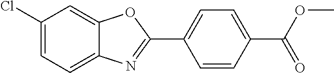 [SMILES: COC(=O)c1ccc(-c2nc3ccc(Cl)cc3o2)cc1] | methyl 4-(6-chlorobenzo[d]oxazol- 2-yl)benzoate |
| 54 | 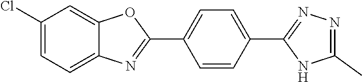 [SMILES: Cc1nnc(-c2ccc(-c3nc4ccc(Cl)cc4o3)cc2)[nH]1] | 6-chloro-2-(4-(5-methyl-4H-1,2,4- triazol-3- yl)phenyl)benzo[d]oxazole |
| 55 67.21 | 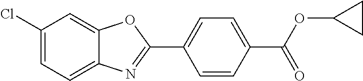 [SMILES: O=C(OC1CC1)c1ccc(-c2nc3ccc(Cl)cc3o2)cc1] | cyclopropyl 4-(6- chlorobenzo[d]oxazol-2- yl)benzoate |
| 56 55.2 | 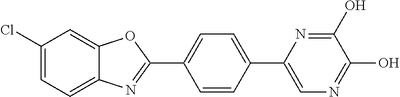 [SMILES: Oc1ncc(-c2ccc(-c3nc4ccc(Cl)cc4o3)cc2)nc1O] | 5-(4-(6-chlorobenzo[d]oxazol-2- yl)phenyl)pyrazine-2,3-diol |
| 57 39.27 | 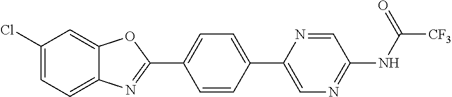 [SMILES: O=C(Nc1cnc(-c2ccc(-c3nc4ccc(Cl)cc4o3)cc2)cn1)C(F)(F)F] | N-(5-(4-(6-chlorobenzo[d]oxazol- 2-yl)phenyl)pyrazin-2-yl)-2,2,2- trifluoroacetamide |
| 58 39.29 | 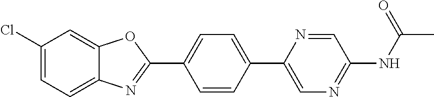 [SMILES: CC(=O)Nc1cnc(-c2ccc(-c3nc4ccc(Cl)cc4o3)cc2)cn1] | N-(5-(4-(6-chlorobenzo[d]oxazol- 2-yl)phenyl)pyrazin-2-yl)acetamide |
| 59 77.44 | 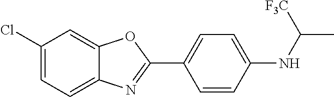 [SMILES: CC(Nc1ccc(-c2nc3ccc(Cl)cc3o2)cc1)C(F)(F)F] | 4-(6-chlorobenzo[d]oxazol-2-yl)-N- (1,1,1-trifluoropropan-2-yl)aniline |
| 60 77.46 | 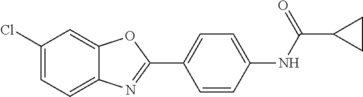 [SMILES: O=C(Nc1ccc(-c2nc3ccc(Cl)cc3o2)cc1)C1CC1] | N-(4-(6-chlorobenzo[d]oxazol-2- yl)phenyl)cyclopropane- carboxamide |
| 61 39.2 | 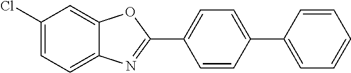 [SMILES: Clc1ccc2nc(-c3ccc(-c4ccccc4)cc3)oc2c1] | 2-([1,l′-biphenyl]-4-yl)-6- chlorobenzo[d]oxazole |
| 62 39.22.5 | 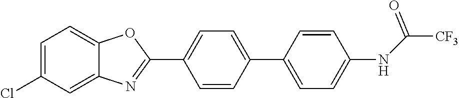 [SMILES: O=C(Nc1ccc(-c2ccc(-c3nc4cc(Cl)ccc4o3)cc2)cc1)C(F)(F)F] | N-(4′-(5-chlorobenzo[d]oxazol-2- yl)-[1,1'-biphenyl]-4-yl)-2,2,2- trifluoroacetamide |
| 63 39.22 | 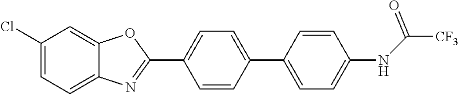 [SMILES: O=C(Nc1ccc(-c2ccc(-c3nc4ccc(Cl)cc4o3)cc2)cc1)C(F)(F)F] | N-(4'-(6-chlorobenzo[d]oxazol-2- yl)-[1,1'-biphenyl]-4-yl)-2,2,2- trifluoroacetamide |
| 64 39.23 | 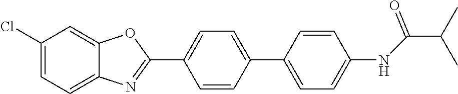 [SMILES: CC(C)C(=O)Nc1ccc(-c2ccc(-c3nc4ccc(Cl)cc4o3)cc2)cc1] | N-(4′-(6-chlorobenzo[d]oxazol-2- yl)-[1,1'-biphenyl]-4- yl)isobutyramide |
| 65 39.24 | 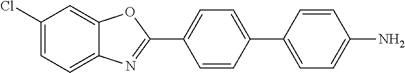 [SMILES: Nc1ccc(-c2ccc(-c3nc4ccc(Cl)cc4o3)cc2)cc1] | 4′-(6-chlorobenzo[d]oxazol-2-yl)- [1,1'-biphenyl]-4-amine |

The CMA Activator can be a compound of Formula III as shown immediately below, or a salt thereof. Compounds of Formula III:

The compound of formula (III) can have the following structures of Table 3:

TABLE 3

| | |
|---|---|
| 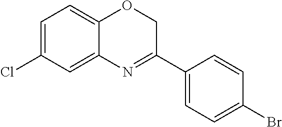 [SMILES: Clc1ccc2c(c1)N=C(c1ccc(Br)cc1)CO2] | 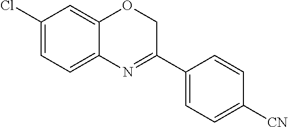 [SMILES: N#Cc1ccc(C2=Nc3ccc(Cl)cc3OC2)cc1] |
| 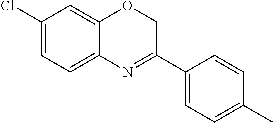 [SMILES: Cc1ccc(C2=Nc3ccc(Cl)cc3OC2)cc1] | 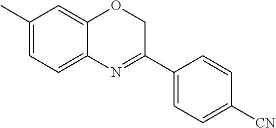 [SMILES: Cc1ccc2c(c1)OCC(c1ccc(C#N)cc1)=N2] |
| 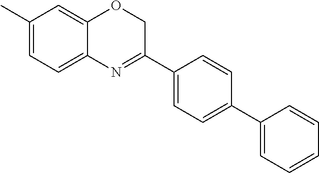 [SMILES: Cc1ccc2c(c1)OCC(c1ccc(-c3ccccc3)cc1)=N2] | 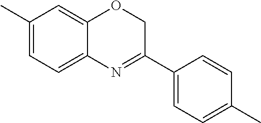 |
| 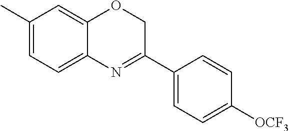 [SMILES: Cc1ccc2c(c1)OCC(c1ccc(OC(F)(F)F)cc1)=N2] | 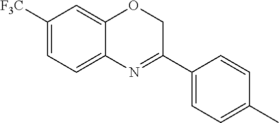 [SMILES: Cc1ccc(C2=Nc3ccc(C(F)(F)F)cc3OC2)cc1] |
| 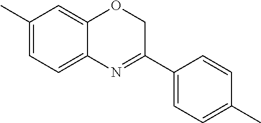 | 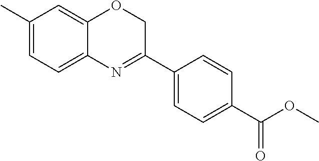 |
| 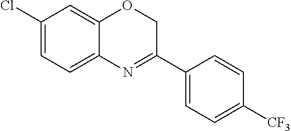 [SMILES: FC(F)(F)c1ccc(C2=Nc3ccc(Cl)cc3OC2)cc1] | 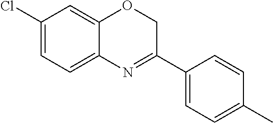 [SMILES: Cc1ccc(C2=Nc3ccc(Cl)cc3OC2)cc1] |
| 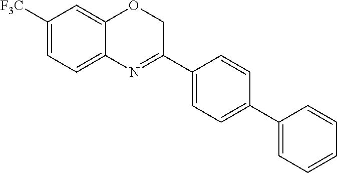 [SMILES: FC(F)(F)c1ccc2c(c1)OCC(c1ccc(-c3ccccc3)cc1)=N2] | 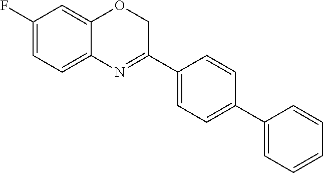 [SMILES: Fc1ccc2c(c1)OCC(c1ccc(-c3ccccc3)cc1)=N2] |
| 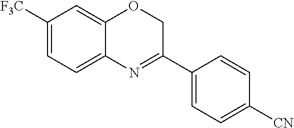 [SMILES: N#Cc1ccc(C2=Nc3ccc(C(F)(F)F)cc3OC2)cc1] | 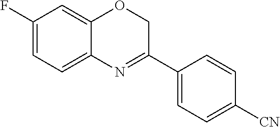 [SMILES: N#Cc1ccc(C2=Nc3ccc(F)cc3OC2)cc1] |
| 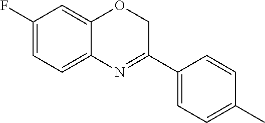 [SMILES: Cc1ccc(C2=Nc3ccc(F)cc3OC2)cc1] | 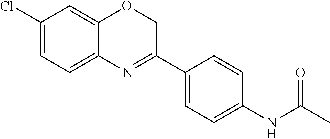 [SMILES: CC(=O)Nc1ccc(C2=Nc3ccc(Cl)cc3OC2)cc1] |
| 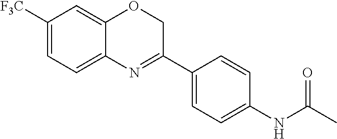 [SMILES: CC(=O)Nc1ccc(C2=Nc3ccc(C(F)(F)F)cc3OC2)cc1] | 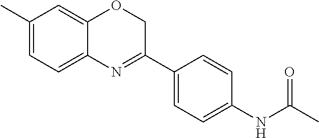 [SMILES: CC(=O)Nc1ccc(C2=Nc3ccc(C)cc3OC2)cc1] |
| 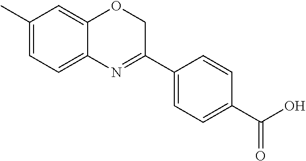 [SMILES: Cc1ccc2c(c1)OCC(c1ccc(C(=O)O)cc1)=N2] | 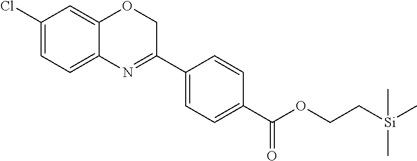 [SMILES: C[Si](C)(C)CCOC(=O)c1ccc(C2=Nc3ccc(Cl)cc3OC2)cc1] |
| 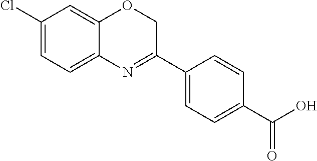 [SMILES: O=C(O)c1ccc(C2=Nc3ccc(Cl)cc3OC2)cc1] | 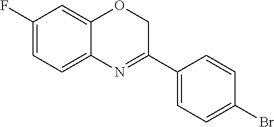 [SMILES: Fc1ccc2c(c1)OCC(c1ccc(Br)cc1)=N2] |
| 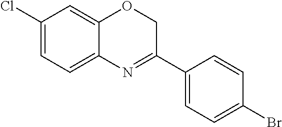 [SMILES: Clc1ccc2c(c1)OCC(c1ccc(Br)cc1)=N2] | 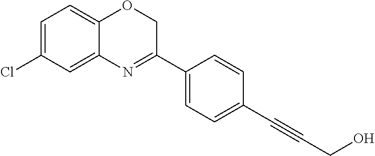 [SMILES: OCC#Cc1ccc(C2=Nc3cc(Cl)ccc3OC2)cc1] |
| 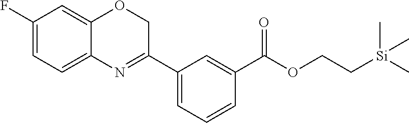 [SMILES: C[Si](C)(C)CCOC(=O)c1cccc(C2=Nc3ccc(F)cc3OC2)c1] | 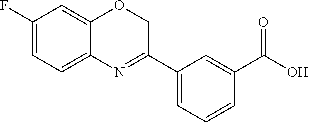 [SMILES: O=C(O)c1cccc(C2=Nc3ccc(F)cc3OC2)c1] |
| 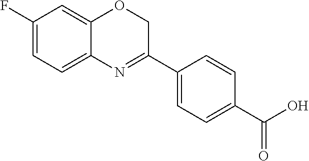 [SMILES: O=C(O)c1ccc(C2=Nc3ccc(F)cc3OC2)cc1] | 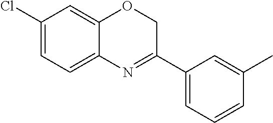 [SMILES: Cc1cccc(C2=Nc3ccc(Cl)cc3OC2)c1] |
| 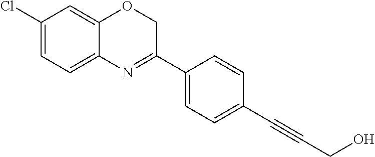 [SMILES: OCC#Cc1ccc(C2=Nc3ccc(Cl)cc3OC2)cc1] | 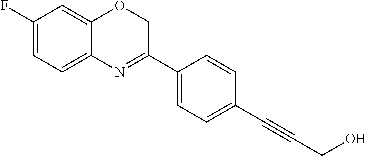 [SMILES: OCC#Cc1ccc(C2=Nc3ccc(F)cc3OC2)cc1] |
| 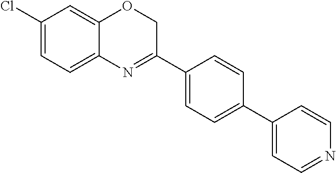 [SMILES: Clc1ccc2c(c1)OCC(c1ccc(-c3ccncc3)cc1)=N2] | 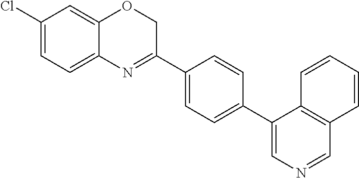 [SMILES: Clc1ccc2c(c1)OCC(c1ccc(-c3cncc4ccccc34)cc1)=N2] |
| 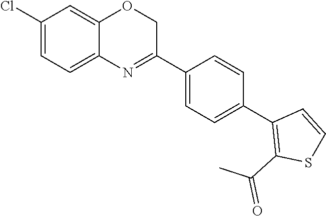 [SMILES: CC(=O)c1sccc1-c1ccc(C2=Nc3ccc(Cl)cc3OC2)cc1] | 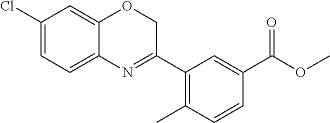 [SMILES: COC(=O)c1ccc(C)c(C2=Nc3ccc(Cl)cc3OC2)c1] |
| 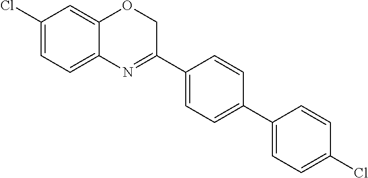 [SMILES: Clc1ccc(-c2ccc(C3=Nc4ccc(Cl)cc4OC3)cc2)cc1] | 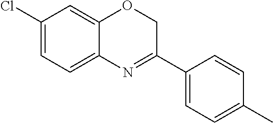 [SMILES: Cc1ccc(C2=Nc3ccc(Cl)cc3OC2)cc1] |

CMA Activators can be administered as the neat chemical but are preferably administered as a pharmaceutical composition. Accordingly, the disclosure provides pharmaceutical compositions comprising a compound or pharmaceutically acceptable salt of a CMA activator, such as a compound of Formula I, II, or III together with at least one pharmaceutically acceptable carrier. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form.

The CMA activator may be administered orally, topically, parenterally, by inhalation or spray, sublingually, transdermally, via buccal administration, rectally, as an ophthalmic solution, through intravitreal injection or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution for topical or intravitreal injection. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present disclosure.

The pharmaceutical compositions/ combinations can be formulated for oral or topical administration. These compositions contain between 0.1 and 99 weight % (wt. %) of a compound of Formula I and usually at least about 5 wt. % of a compound of Formula I, II, or III. Some embodiments contain from about 25 wt. % to about 50 wt. % or from about 5 wt. % to about 75 wt. % of a compound of Formula I, II, or III.

### METHODS OF TREATMENT

The disclosure also provides methods of selectively activating chaperone-mediated autophagy (CMA) in a mammal in need thereof comprising administering to the mammal a compound of Formula I, II, or III in an amount effective to activate CMA in the mammal.

A therapeutically effective amount of a CMA Activator is an amount sufficient to inhibit the progression of a disease or disorder, cause a regression of a disease or disorder, reduce symptoms of a disease or disorder, or significantly alter a level of a marker of a disease or disorder, or reduce the probability of a treated mammal of developing the disease or disorder relative to an untreated mammal of the same species.

The disclosure includes methods in which the blood cancer is leukemia, lymphoma or myeloma. Leukemia is a blood cancer that originates in the blood and bone marrow. Leukemia can be of various types for example, Acute Lymphoblastic Leukemia, Acute Myeloid Leukemia, Chronic Lymphocytic Leukemia, Chronic Myeloid Leukemia, Hairy Cell Leukemia, Chronic Myelomonocytic Leukemia, Juvenile Myelomonocytic Leukemia, Large Granular Lymphocytic Leukemia, Blastic Plasmacytoid Dendritic Cell Neoplasm, B-cell Prolymphocytic Leukemia, T-cell Prolymphocytic Leukemia, and others.

Non-Hodgkin lymphoma is a blood cancer that develops in the lymphatic system from cells called lymphocytes, a type of white blood cell that helps the body fight infections. Hodgkin lymphoma is a blood cancer that develops in the lymphatic system from cells called lymphocytes. Hodgkin lymphoma is characterized by the presence of an abnormal lymphocyte called the Reed-Sternberg cell.

Multiple myeloma is a blood cancer that begins in the blood's plasma cells, a type of white blood cell made in the bone marrow.

In addition to the methods of treatment and increasing proteostasis provided in the SUMMARY section this disclosure also provides such methods in which (i) the mammal treated is a human, aged 55 years or more, aged 60 years or more, aged 65 years or more, aged 70 years or more, aged 75 years or more, or aged 80 years or more, (ii) the mammal is a companion animal (such as a dog or cat) or a livestock animal (such as cow, pig, sheep, goat, or horse), (iii) the mammal is suffering from myeloid leukemia, (iv) the mammal has a blood cancer and the blood cancer is acute myeloid leukemia, (v) the mammal has a myelodysplastic syndrome or is at risk for myelodysplastic syndrome (vi) the mammal has myelodysplastic syndrome and the myelodysplastic syndrome is a syndrome of low red blood cell count, such as anemia, a syndrome of low white cell count, such as neutropenia, or a syndrome of low platelet count such as thrombocytopenia, (vii) wherein the blood cells are red blood cells, (viii) the blood cells are white blood cells, such as neutrophils, (ix) the blood cells are platelets, (x) the CMA activator is a compound of Formula I, Formula II, or Formula III as disclosed in the specification, and/or (xi) the CMA activator is a compound of Table 1, Table 2, of a specific compound of Formula III as disclosed in the specification, or a salt of a compound or Table 1, Table 2, of a salt of a specific compound of Formula III as disclosed in the specification.

### TERMINOLOGY

The terms “a” and “an” do not denote a limitation of quantity, but rather denote the presence of at least one of the referenced items. The term “or” means “and/or”. The open-ended transitional phrase “comprising” encompasses the intermediate transitional phrase “consisting essentially of” and the close-ended phrase “consisting of ” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to thepractice of the invention as used herein. Unless defined otherwise, technical and scientific terms used herein have the same meaning as is commonly understood by one of skill in the art to which this invention belongs.

As used herein, the term “differentiation” as used with respect to cells in a differentiating cell system refers to the process by which cells differentiate (change) from one cell type (e.g., a multipotent, totipotent, or pluripotent differentiable cell) to another cell type such as a target differentiated cell. As used herein, the term “cell differentiation” in reference to a pathway refers to a process by which a less specialized cell (i.e. stem cell) develops or matures (becomes more phenotypically specified) or differentiates to possess a more distinct form and/or function into a more specialized cell or differentiated cell.

As used herein, the term “default” or “passive” in reference to a cell differentiation pathway refers to a pathway where a less specialized cell becomes a certain/specific differentiated cell type in culture, when not treating with certain compounds i.e. normal cell cultures conditions. In other words, a default cell results when a cell is not contacted by a molecule capable of changing the differentiated cell type (i.e. a morphogen). In contrast, “non-default” in reference to a cell refers to a differentiated cell type that results that is different from a default cell, i.e. a non-default cell is a differentiated cell type resulting from a non-default condition,

As used herein, the term “contacting” cells with a compound of the present inventions refers to placing the compound in a location that will allow it to touch the cell to produce “contacted” cells. The contacting may be accomplished using any suitable method. For example, in one embodiment, contacting is by adding the compound to a tube of cells. Contacting may also be accomplished by adding the compound to a culture of the cells.

As used herein, the term “stem cell” refers to a cell that is totipotent or pluripotent or multipotent and can differentiate into one or more different cell types.

As used herein, the tern “pluripotent” refers to a cell line capable of differentiating into any differentiated cell type.

As used herein, the term “multipotent” refers to a cell line capable of differentiating into at least two differentiated cell types.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell cultures.

The term “in vivo” refers to the natural environment (e.g., an animal or a cell) and to processes or reaction that occur within a natural environment.

As used herein, the term “progenitor” in reference to a cell or an area of cells refers to the type of cell or area of cells that would develop (differentiate into) under the appropriate conditions, i.e. when contacted with a proper growth factor, compound, extracellular signal, intracellular signal, etc.

As used herein, the term “attached cell” refers to a cell growing in vitro wherein the cell contacts the bottom or side of the culture dish, an attached cell may contact the dish via extracellular matrix molecules and the like. As opposed to a cell in a suspension culture.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type. As used herein, the term “test compound” refers to any chemical entity, pharmaceutical, drug, and the like that were used to provide cells of the present inventions.

As used herein, the term “increasing” in reference to a characteristic refers to a larger amount of a characteristic when compared to said characteristic in a control.

As used herein, the term “decreasing” in reference to a characteristic refers to a smaller amount of a characteristic when compared to said characteristic in a control.

The term “sample” is used in its broadest sense. In one sense it can refer to a cell or tissue. In another sense, it is meant to include a specimen or culture obtained from any source and encompass fluids, solids, and tissues. These examples are not to be construed as limiting the sample types applicable to the present invention.

The terms “purified,” “to purify,” “purification,” “isolated,” “to isolate,” “isolation,” and grammatical equivalents thereof as used herein, refer to the reduction in the amount of at least one contaminant from a sample. For example, a cell type is purified by at least a 10%, preferably by at least 30%, more preferably by at least 50%, yet more preferably by at least 75%, and most preferably by at least 90%, reduction in the amount of undesirable cell types.

As used herein, the term “proliferation” refers to an increase in cell number.

As used herein, the term “ligand” refers to a molecule that binds to a second molecule. A particular molecule may be referred to as either, or both, a ligand and second molecule. Examples of second molecules include a receptor of the ligand, and an antibody that binds to the ligand.

The term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vivo, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “biologically active,” refers to a molecule (e.g. peptide, nucleic acid sequence, carbohydrate molecule, organic or inorganic molecule, and the like) having structured, regulatory, and/or biochemical functions.

As used herein, the term “primary cell” is a cell that is directly obtained from a tissue (e.g. blood) or organ of an animal in the absence of culture. Typically, though not necessarily, a primary cell can undergo ten or fewer passages in vitro before senescence and/or cessation of proliferation. In contrast, a “cultured cell” is a cell that has been maintained and/or propagated in vitro for ten or more passages.

As used herein, the term “cultured cells” refer to cells that are capable of a greater number of passages in vitro before cessation of proliferation and/or senescence when compared to primary cells from the same source. Cultured cells include “cell lines” and “primary cultured cells.”

As used herein, the term “cell culture” refers to any in vitro culture of cells. Included within this term are continuous cell lines (e.g. with an immortal phenotype), primary cell cultures, finite cell lines (e.g., non-transformed cells), and any other cell population maintained in vitro.

As used herein, the term “cell line,” refers to cells that are cultured in vitro, including primary cell lines, finite cell lines, continuous cell lines, and transformed cell lines, but does not require, that the cells be capable of an infinite number of passages in culture. Cell lines may be generated spontaneously or by transformation.

As used herein, the terms “primary cell culture,” and “primary culture,” refer to cell cultures that have been directly obtained from cells in vivo, such as from animal tissue. These cultures may be derived from adults as well as fetal tissue.

As used herein, the terms “monolayer,” “monolayer culture,” and “monolayer cell culture,” refers to a cell that has adhered to a substrate and grow as a layer that is one cell in thickness, in other words, an “attached cell.” Monolayers may be grown in any format, including but not limited to flasks, tubes, coverslips (e.g., shell vials), roller bottles, et cetera.

As used herein, the terms “feeder cell layer” or “feeder cell population” refers to a monolayer of cells used to provide attachment molecules and/or growth factors for an adjacent cell, for example, used in co-culture to maintain pluripotent stem cells. As used herein, the terms “suspension” and “suspension culture” refer to cells that survive and proliferate without being attached to a substrate. Suspension cultures are typically produced using hematopoietic cells, transformed cell lines, and cells from malignant tumors.

As used herein, the terms “culture media,” and “cell culture media,” refer to media that are suitable to support the growth of cells in vitro (i.e., cell cultures, cell lines, etc.). It is not intended that the term be limited to any particular culture medium. For example, it is intended that the definition encompass outgrowth as well as maintenance media. Indeed, it is intended that the term encompass any culture medium suitable for the growth of the cell cultures and cells of interest.

As used herein, the term “cell” refers to a single cell as well as to a population of (i.e., more than one) cells. The population may be a pure population comprising one cell type, such as a population of neuronal cells or a population of undifferentiated embryonic cells. Alternatively, the population may comprise more than one cell type, for example a mixed cell population. It is not meant to limit the number of cells in a population, for example, a mixed population of cells may comprise at least one differentiated cell. In one embodiment a mixed population may comprise at least one differentiated. In the present inventions, there is no limit on the number of cell types that a cell population may comprise.

The term “compounds of Formula I” (or ‘Formula II or Formula III) encompasses all compounds that satisfy Formula I (or Formula II or Formula III, respectively), including any enantiomers, racemates and stereoisomers, as well as all pharmaceutically acceptable salts of such compounds. A dash (“-”) that is not between two letters or symbols is used to indicate a point of attachment for a substituent.

A dash (“-”) that is not between two letters or symbols is used to indicate a point of attachment for a substituent. For example, —(C═O)OH is attached through carbon of the keto(C═O) group.

A bond represented by a combination of a solid and dashed line, i.e., [symbol] may be either a single or double bond.

“Alkyl” is a branched or straight chain or cyclic saturated aliphatic hydrocarbon group, having the specified number of carbon atoms, generally from 1 to about 8 carbon atoms. The term C1-C6alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₄alkyl and C₁-C₂alkyl. When C₀-Cₙalkyl is used herein inconjunction with another group, for example, -C₀-C₀-C₂alkyl(phenyl), the indicated group, in this case phenyl, is either directly bound by a single covalent bond (C₉alkyl), or attached by an alkyl chain having the specified number of carbon atoms, in this case 1, 2, 3, or 4 carbon atoms.

Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, cyclopropyl, cyclopropylmethyl, cyclopropylethyl, n-butyl, cyclobutyl, 3-methylbutyl,t-butyl, cyclobutyl methyl. n-pentyl, and sec-pentyl.

“Alkoxy” is an alkyl group as defined above with the indicated number of carbon atomscovalently bound to the group it substitutes by an oxygen bridge (—O—).

Examples of alkoxy include, but are not limited to, methoxy, ethoxy, n-propoxy, i-propoxy, cyclopropyloxy, cyclopropylmethoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Aryl” indicates aromatic groups containing only carbon in the aromatic ring or rings. Typical aryl groups contain 1 to 3 separate, fused, or pendant rings and from 6 to about 18 ringatoms, without heteroatoms as ring members. When indicated, such aryl groups may be furthersubstituted with carbon or non-carbon atoms or groups. Aryl groups include, for example, phenyl, naphthyl, including 1-naphthyl, 2-naphthyl, and bi-phenyl.

“Cycloalkyl” is a saturated hydrocarbon ring group, having the specified number of carbon atoms. Monocyclic cycloalkyl groups typically have from 3 to about 7 (3, 4, 5, 6, or 7) carbon ring atoms. Cycloalkyl substituents may be pendant from a substituted nitrogen, sulfur, oxygen or carbon atom, or a substituted carbon atom that may have two substituents may have acycloalkyl group, which is attached as a spiro group. Examples of cycloalkyl groups include cyclopropyl, cyclobutyl, cyclopentyl, or cyclohexyl as well as bridged or caged saturated ring groups such as norbomane or adamantine.

“Haloalkyl” includes both branched and straight-chain alkyl groups having the specifiednumber of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and pentafluoroethyl.

“Haloalkoxy” is a haloalkyl group as defined herein attached through an oxygen bridge(oxygen of an alcohol radical).

“Halo” or “halogen” indicates any of fluoro, chloro, bromo, and iodo.

“Heteroaryl” is a stable monocyclic aromatic ring having the indicated number of ring atoms which contains from 1 to 4, or in some embodiments from 1 to 2, heteroatoms chosen from N, O, and S, with remaining ring atoms being carbon, or a stable bicyclic system containingat least one 5- to 7-membered aromatic ring which contains from 1 to 4, or in some embodimentsfrom 1 to 2, heteroatoms chosen from N, O, and S, with remaining ring atoms being carbon.

Monocyclic heteroaryl groups typically have from 5 to 7 ring atoms. In certain embodiments theheteroaryl group is a 5- or 6-membered heteroaryl group having 1, 2, 3, or 4 heteroatoms chosenfrom N, O, and S, with no more than 2 O atoms and 1 S atom.

The term “substituted,” as used herein, means that any one or more hydrogens on the designated atom or group is replaced with a selection from the indicated group, provided that thedesignated atom's normal valence is not exceeded. When the substituent is oxo (i.e., ═O ) then 2 hydrogens on the atom are replaced. When an oxo group substitutes aromatic moieties, the corresponding partially unsaturated ring replaces the aromatic ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Unless otherwise specified substituents are named into the core structure. For example, it is to beunderstood that when aminoalkyl is listed as a possible substituent the point of attachment of thissubstituent to the core structure is in the alkyl portion.

In certain embodiments, groups that may be “substituted” or “optionally substituted” include, but are not limited to: monocyclic aryl, e.g., phenyl; monocyclic heteroaryl, e.g., pyrrolyl, pyrazolyl, thienyl, furanyl, imidazolyl, thiazolyl, triazolyl, pyridyl, pyrimidinyl; bicyclicheteroaryl, e.g., benzimidazolyl, imidazopyridizinyl, indolyl, indazolyl, quinolinyl, isoquinolinyl; and C₁-C₆alkyl in which any carbon-carbon single bond is optionally replaced by a carbon-carbon double or triple bond, any methylene group is optionally replaced by O, S, or NR¹².

Suitable groups that may be present on a “substituted” or “optionally substituted” position include, but are not limited to: halogen; cyano; CHO; COOH; hydroxyl; oxo; amino; alkyl groups from 1 to about 6 carbon atoms; alkoxy groups having one or more oxygen linkagesand from 1 to about 8, or from 1 to about 6 carbon atoms; haloalkyl groups having one or more halogens and from 1 to about 8, from 1 to about 6, or from 1 to about 2 carbon atoms; and haloalkoxy groups having one or more oxygen linkages and one or more halogens and from 1 to about 8, from 1 to about 6, or from 1 to about 2 carbon atoms.

“Pharmaceutical compositions” are compositions comprising at least one active agent, such as a CMA Activator, for example a compound or salt of Formula I, II, or III, and at least one other substance, such as a carrier.

Pharmaceutical compositions optionally contain one or more additional active agents. When specified, pharmaceutical compositions meet the U.S. FDA's GMP (good manufacturing practice) standards for human or non-human drugs.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in amixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example,from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic,propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC-(CH₂)ₙ—COOH where n is 0-4, and the like.

The term “carrier” applied to pharmaceutical compositions/ combinations of the presentdisclosure refers to a diluent, excipient, or vehicle with which an active compound is provided. To be pharmaceutically acceptable a carrier must be safe, non-toxic and neither biologically norotherwise undesirable.

The term “prevention” encompasses prevention of at least one symptom or other embodiment of a disorder or disease. A prophylactically administered treatment incorporating a CMA activator, culture media, and so forth, of the disclosure, need not be completely effective in preventing the onset of a disease or disorder to constitute a viable prophylactic agent. Simply reducing the likelihood that the disease, disorder, or symptom will occur or worsen in a subject, is sufficient.

The term “treatment” encompasses alleviation of at least one symptom of a disease or disorder, or reduction of disease severity, and the like. A treatment need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method comprising administering to a patient a CMA activator in an amount and for a time sufficient to induce a sustained improvement over baseline of an indicator of a skin disease or disorder.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1. General Methods

Mice. Hematopoietic system specific L2A KO mice were created by crossing C57BL/6 Vav-iCre mice with C57BL/6 L2Af/f mice. Wild type, Vav-iCre and L2Af/f40 male littermate mice were separately analyzed for each test and because no differences were detected among them, they were grouped in the results as “control” for the experimental group (Vav-iCreL2Af/f). For the aging studies, 3-4 months (labeled as 4 m) and 25-30 m (labeled as >25 m) male mice were used in the young and old group, respectively. KFERQ-Dendra2 transgenic mice and KFERQ-Dendra mice defective in CMA (KFERQ-Dendra-L2AKO) were generated as described before. Transgenic mice with a targeted insertion of a human copy of L2A that can be induced by tamoxifen (hL2AOE) were generated by crossing hL2Af/f mice with TmxER-Cre71. hLAMP2A expression was induced with 3 intraperitoneal (i.p.) injections of tamoxifen (2 mg/20 g body weight) every other day either at 4 m and analyzed at 7m of age or at 12m and analyzed at 27 m of age. Young and old controls were similarly injected with tamoxifen. Injected animals did not show differences in HSC frequency with non-injected controls, thus discarding any possible direct effect of tamoxifen at the time that the animals were analyzed. KFERQ-Dendra mice with extra copy of human L2A (KFERQ-Dendra-hL2AOE) were generated by breeding KFERQ-Dendra male mice with hL2AOE mice. Where indicated, mice were i.p. injected with a single dose of 5-fluorouracil (SFU, 150 mg/kg body weight, Invitrogen, sud-5 fu). Blood cell count was analyzed in tail blood drawn at days 0, 1, 3, 6, 8, and 16 post 5 FU injection using an Oxford Science Forcyte Blood Analysis Unit. Bone marrow was analyzed at the same times. Data is from the analysis 8 days post-injection, unless otherwise specified. For serial 5FU injections, mice were injected every week and 7 days post injection blood was taken and blood counts measured as above and dead mice and time of death were registered. For GLA supplementation, old mice (>25 m) were injected with either saline or GLA (1mg/kg bw) for 40 days. The CMA activator (CA 20 mg/Kg b.w. per mouse day for 2 months) was administered orally in the form of sucralose gelatin agar pills. The same delivery method was utilized in control mice but using drug-free pills. CA is a derivative from the previously generated first-in-class small molecules for selective activation of CMA in vitro 61, that has been modified to make it suitable for in vivo administration (Gomez et al., in preparation). All mouse procedures were approved by the Institutional Animal Care and Use Committee of Albert Einstein College of Medicine.

Human hematopoietic stem and progenitor cells. Mobilized peripheral blood from multiple myeloma patients (59-71 years old) was enriched for mononuclear cells by density gradient centrifugation using Ficoll-Paque PLUS (GE Healthcare). Mono-nuclear cells were further enriched for CD34+ cells using immunomagnetic bead sorting (CD34 MicroBead Kit, human; Miltenyi Biotec) according to the manufacturer's protocol. The isolated CD34+cells were then used for LTC-IC or colony formation assays.

Competitive bone marrow transplantation. Total nucleated bone marrow (BM) cells (5×10 5) were injected retro-orbitally into lethally irradiated CD45.1 congenic mice in competition with the same amount of BM cells from CD45.1 mice. Transplanted mice were given antibiotic-containing water for 4 weeks post-irradiation. Reconstitution of donor derived (CD45.2) cells was monitored by staining blood cells with antibodies against CD45.2 and CD45.1 at different times as indicated. For serial transplantation, donor-derived BM cells were collected from recipient mice 24 weeks after first BM transplantation. Cells were then transplanted into recipient mice with fresh competitor cells (CD45.1).

Flow cytometry. For flow cytometric analyses of mouse cells, the following monoclonal antibodies were used: APC-CD117(c-kit) (Biolegend 105812), PE/Cy7-Ly-6A/E (Sca-1) (Biolegend 108114), Pacific blue-CD48 (Biolegend 103418), PE-CD150 (Biolegend 115904), APC-CD3E (Biolegend 100312), Percp/Cy5.5-B220 (Biolegend 103236), FITC-CD11b (Biolegend 101206), FITC-Ly-6G/Ly-6C (Gr-1) (Biolegend 108406), and FITC-CD34 (eBioscience, 11-0341-82), PE-CD16/32 (Biolegend 101308). Biotin-conjugated antibodies for lineage markers were as follows: CD3 (Biolegend 100244), CD4 (Biolegend 100404), CD8a (Biolegend 100704), IgM (Biolegend 406504), TER-119 (Biolegend 116204), B220 (Biolegend 103204), NK-1.1 (Biolegend 108704), CD19 (Biolegend, 115504). PerCP/Cy5.5 Streptavidin (Biolegend, 405214) was used against the biotin-conjugated antibodies. Before analysis, BM from tibias and femurs were depleted from red blood cells with ACK lysis buffer, then stained with HSC markers (Lin−/cKit+/Scal+/CD48−/CD150+) or myeloid progenitor markers.

(Lin−/cKit+/Scal−) or GMP markers (Lin−/cKit+/Scal−/CD34hi/CD16/32hi) followed by either ROS staining or cell cycle analysis. All antibodies used in this study were from commercial sources and were validated following the multiple dilution method and, where available, using cell lines or tissues from animals knock-out for the antigen. For ROS staining, cells were resuspended in staining buffer (2% FBS/2 mM EDTA in PBS) with 5 μM CellROX reagent (ThermoFisher, C10444) and incubated at 37° C. for 30 min. Cells were washed 3 times with staining buffer, resuspended in the same buffer with 1 μg/ml propidium iodide for dead cell exclusion and then subjected to FACS analysis. For cell cycle analysis, after staining for cell surface markers, cells were fixed with Cytofix/Cytoperm buffer (BD Bioscience, 554722) for 20 min at 4° C., washed twice with Perm/Wash buffer (BD Bioscience, 554723), re-suspended in Perm/Wash buffer containing Alexa Fluor 488 anti-mouse Ki-67 antibody (Biolegend, 652417) and incubated overnight at 4° C. After 3 washes with Perm/Wash buffer, cells were resuspended in cell staining buffer with Hoechst 33342 (ThermoFisher, H3570) for 5 min at RT, and subjected to FACS analysis. For BrdU staining, mice were intraperitoneally injected with a single dose of 100 mg/kg BrdU (Sigma-Aldrich, B5002) and provided with drinking water containing 0.8 mg/ml BrdU and 5% glucose 24 hours before dissection. After staining with surface markers, cells were fixed, permeabilized, treated with DNase, and stained with anti-BrdU FITC antibody (Biolegend, 364103) following the BrdU labeling kit protocol (BD Pharmingen). Cells were then resuspended in staining buffer with Hoechst 33342. For sorting, BM cells from tibias, femurs and spine were centrifuged through Histopaque to isolate mononucleated cells. Cells were stained with HSC markers or LSK markers, resuspended in the staining buffer with 1 μg/ml propidium iodide for dead cell exclusion and then subjected to sorting in a Beckman Moflo sorter with 70 μm nozzle. Data was acquired and analyzed with the BD FACSDiva™ software.

Colony formation and serial re-plating assays. Sorted mouse HSC (180 cells Lin−/cKit+/Scal+/CD48−/CD150+) or CD34-enriched human HSPC (5,000 cells) were plated into methylcellulose media (STEMCELL Technologies, MethoCult M 3434 for mouse or MethoCult H4434 for human) according to the manufacturer's protocols. Colonies were scored after 10 to 12 days for mouse and 14 to 16 days for human cells using an Inverted Infinity and Phase Contrast Microscope (Fisher Scientific). For serial re-plating assays, methylcellulose medium from primary platings were dissolved in PBS to dissociate the colonies into a single-cell suspension, washed three times and 20,000 cells (mouse) or 40,000 cells (human) were re-plated in lml of MethoCult M3434 or MethoCult H4434 medium for mouse and human cells, respectively. Where indicated, media was supplemented with 100 nM FADS2 inhibitor SC-26196 (Sigma, PZ0176), 100 μM γ-linolenic fatty acid (GLA) (Sigma, L2378), 100 μtM NAC (Sigma, A7250) or 5 mM Methyl-pyruvate (Sigma, 371173).

LTC-IC assay. To assess LTC-IC frequencies, serial dilutions of FACS-sorted mouse Lin−Sca-l+c-Kit+ (LSK) cells or CD34+enriched human HSPCs were plated in MyeloCult M5300 for mouse or MyeloCult H5100 for human (STEMCELL Technologies) mixed at 50:50 ratio with primary mouse stroma cell—conditioned medium or with HS5-conditioned medium for human cells. For each cell dose (1:10, 1:20, 1:40, 1:80, 1:160 for mouse LSK cells, 1:50, 1:100, 1:200, 1:400, 1:800 for human HSPC), 10 technical replicates were performed. After 4 weeks (mouse) or 5 weeks (human) in culture with half change of the medium every week, cells in each well were used for the colony formation assay in MethoCult M3434 and MethoCult H4434 (STEMCELL technologies) for mouse and human, respectively. For CFU-C counting, in the case of mouse samples, wells containing any type of colonies were scored as positive, while in the case of human samples, only wells with GEMM colonies were scored as positive. Where indicated, the CMA activator (10 μM) or the same volume of DMSO were added to the mouse LSK cells every 24 hours during the 4 weeks in culture. Supplementation of the γ-linolenic fatty acid (GLA) was done by adding 100 μtM GLA (Sigma, L2378) or the same volume of IMDM medium to the human HSPC cells every week for 5 weeks.

Long-term ex vivo expansion of mouse hematopoietic stem cells and electroporation. The long-term ex vivo expansion of mouse HSC were conducted as previously described. Briefly, Ctrl or L2AKO HSC were sorted into a well of 96-well plates (3 plates for each genotype) containing F-12 medium supplemented with 10 mM HEPES, 1×PSG, 1×ITSX, 1 mg/ml PVA, 100 ng/ml TPO, and 10 ng/ml SCF. Cells were cultured for one month with half of the medium changed twice a week. Cells were then collected and subjected to gradient centrifugation using Ficoll (Sigma, Histopaque-1083) to remove any dead cells. Electroporation was performed using the Amaxa Human CD34+ cell Nucleofector kit (Lonza, VPA-1003) according to the manufacturer's instructions. Cells (2×10⁶) were electroporated with 3 μg DNA coding for FADS2, FADS2-K42Q or FADS2-K42A together with 1.2 μg of the plasmid pmaxGFP. Neomycin (0.1 mg/ml) was used for selection after 48 hours of electroporation and cells were cultured for another 3 weeks following the protocol for long-term ex vivo expansion. Twenty-four hours before the experiment, cells were sorted for HSC (CD48-CD150+LSK) and then treated with leupeptin (100 μM) and NH4Cl (20 mM) for 16 hours and collected for SDS-PAGE and immunoblot.

Seahorse assay. Oxygen consumption rates and extracellular acidification rates were measured using a 96-well Seahorse Bioanalyzer XF 96 according to the manufacturer's instructions (Agilent Technologies). In brief, LSK cells were sorted and plated into 96-well plates pre-coated with CELL-TAK (CORNING, 354240). For extracellular acidification rates to measure glycolysis, cells (100,000 cells per well) were plated into 180 μl base media (100 nM SCF, 100 nM TPO, 2 mM L-Glutamine, 1 mM Pyruvate), spin down at 80 g for 1 min and incubated within a CO2-free chamber at 37° C. for 1 hour. Once in the reader, plates were sequentially injected with 30 mM glucose, 2 μM oligomycin and 100 mM 2-DG or just oligomycin and 2-DG where is indicated in the related figures. To determine the fraction of oxygen consumption dependent on fatty acid β-oxidation, cells (200,000 cells well) were plated into 180 μl KHB media (111 mM NaCl, 4.7 mM KCl, 1.25 mM CaCl₂, 2 mM MgSO₄, 1.2 mM NaH₂PO₄) and in half of the samples etomoxir (40 μM, Sigma, E1905) was added for 30 min before the analysis. Glycolysis was calculated as ECAR rate after adding saturating amount of glucose, maximal glycolytic capacity as maximum ECAR rate reached upon addition of oligomycin and glycolytic reserve as the difference between glycolytic capacity and glycolysis rate. Once in the reader, plates were sequentially injected with 1 μM oligomycin, 2 μM FCCP and 0.5 μM Rotenone. Fatty acid β-oxidation rate was calculated as the difference in oxygen consumption in presence or absence of etomoxir. Data were normalized to cell number using CyQuant (ThermoFisher, C7026).

Enzyme activity and ATP measurement. Activity of Pyruvate Kinase or GAPDH were measured using Biovision kit (K709 for Pyruvate Kinase, K608 for GAPDH) according to the manufacturer's instructions. Briefly, for Pyruvate Kinase, freshly sorted LSK cells (50,000) were collected by centrifugation, lysed with 50 μl lysis buffer and after centrifugation, the supernatant was added to 96-well plate with clear bottom, followed by 50 μl reaction mix. The plate was read using fluorescence Ex/Em=535/587 nm every 10 minutes. The Pyruvate Kinase activity was calculated by the two readings within a linear range. For the GAPDH activity, LSK cells (50,000) were lysed with 25 μl GAPDH Assay buffer by incubation on ice for 10 min followed by centrifugation. The supernatant (20 μl per well) adjusted to a final volume of 50 μl with GAPDH Assay Buffer was incubated with 50 μl of reaction mix and the plate measured at 450 nm in kinetic mode for 10-60 min at 37° C. ATP was measured using ATPlite luminescence ATP detection assay kit (PerkinElmer). Briefly, HSC (10,000) were plated in a white bottom 96-well plate with 100 μ2% FBS in PBS and 50 μl mammalian cell lysis solution for 5 min with shaking, followed by additional 5 min incubation with 50 μl substrate solution. Plates were measured in a luminometer.

Immunofluorescence staining. HSC or myeloid progenitor cells were directly sorted into 16-well slides pre-coated with Cell-Tak Cell Tissue Adhesive (Coming, 354240) and then fixed with 4% PFA for 15 min at room temperature (RT). For LAMP2A, LAMP1, total human LAMP2 and FADS2 staining, slides were washed with PBS and incubated with blocking buffer (5% goat serum/0.3% TrionX-100 in PBS) for 1 hour at RT and incubated ovemight at 4° C. with the first antibody diluted in 1% BSA/0.3% TritonX-100 followed by 40 min incubation at RT with fluorescence-conjugated secondary antibodies. Cells were washed 3 times with PBS and mounted with mounting medium with DAPI (Southern biotech). For LC3 staining, after fixation, cells were permeabilized with 0.015% (v/v) digitonin in PBS (Sigma) and then incubated with blocking buffer (10% FBS in PBS) for 45 minutes. Both the 1st and 2nd antibody were diluted in blocking buffer and incubated for 30 minutes and 45 minutes at RT, respectively. Oxidized proteins were detected with OxyICC Oxidized Protein Detection Kit from Sigma (S7350) and protein inclusions were detected with PROTEOSTAT Aggresome detection kit from ENZO (ENZ-51035) following manufacturer's instructions. For LysoTracker staining, cells were incubated with 50 nM LysoTracker green (Invitrogen, L7526) for 30 min at 37° C., washed and fixed for 10 minutes with 4% PFA and mounted. For analysis of CMA activity using direct fluorescence and the KFERQ-Dendra CMA reporter, cells isolated from KFERQ-Dendra2 transgenic mice were fixed with 2% PFA for 5 min at RT and mounted for direct puncta counting or subjected for immunofluorescence as described above. Quantification was performed in TIFF converted images upon thresholding using the 3D object counter tool of Image J software (NIH). Average number of puncta per cell was determined for each of the cells in a field and at least 3 different fields per animal were counted. Where indicated, we also calculated the percentage of cells active for CMA, defined as those with at least two Dendra positive puncta. To determine CMA flux using the CMA reporter, cells were incubated or not in the presence of leupeptin (100 μM, 16 hours), fixed with 4% PFA for 15 minutes followed by blocking with 5% goat serum/0.01% tritonX-100 for 1 hour at RT, and then incubated with the dendra2 antibody and the corresponding secondary antibody in 1% BSA/0.01% TritonX-100 in PBS sequentially. CMA flux was calculated as the increase in number of Dendra positive puncta upon leupeptin treatment. The following primary antibodies were used: anti-mouse LAMP2A (Invitrogen, 512200), anti-mouse LAMP1 (Hybridoma Bank, 1D4B), total human LAMP2 (Hybridoma Bank, h4b4), FADS2 (Abclonal, A10270), LC3 (MBL, PM036), Dendra2 (antibodies, abin361314).The following secondary antibodies were used: Alexa Fluor 488 goat anti-rabbit IgG (H+L) (Invitrogen, A-11008) and Alexa Fluor 488 goat anti-rat IgG (H+L) (Invitrogen, A-11006), Alexa Fluor 555 goat anti-rabbit IgG (Invitrogen, A-21428), Alexa Fluor 555 goat anti-rat IgG (Invitrogen, A-21434), Alexa Fluor 635 goat anti-rabbit IgG (Invitrogen, A-31576). All the images were taken with a Confocal microscope (TCS SP8; Leica) using an HCX Plan Apo CS 63.0×1.40 NA oil objective and the Leica Application Suite X (LAS X).

Transmission Electron microscopy. Cells (100,000 per condition) were pelleted and fixed with 2% paraformaldehyde and 2.5% glutaraldehyde in 0.1M sodium cacodylate buffer, followed by sequential fixation in 1% osmium tetroxide followed and 2% uranyl acetate. Samples were dehydrated through a graded series of ethanol dilutions and embedded in LX112 resin (LADD Research Industries). Ultrathin (80 nm) sections were cut on a Leica EM Ultracut UC7, stained with uranyl acetate followed by lead citrate, and viewed on a JEOL 1200EX transmission electron microscope at 80 kV.

RNA Purification, Amplification, and Microarray Analysis. Cells (10,000) were sorted, and total RNA was extracted using RNeasy Micro kit (Qiagen) according to the manufacturer's protocols. All total RNA samples were quantified with the RNA Quantification Kit (ThermoFisher Scientific, 902905). Using the GeneChip WT Pico Kit (ThermoFisher Scientific, 902622), samples were amplified to cRNA from in vitro transcription of one round of linear amplification reaction and then converted to sense-strand single stranded cDNA followed fragmentation of biotinylated cDNA. 5.5 micrograms of cDNA from each sample was hybridized to ThermoFisher Scientific (Affymetrix) GeneChip Mouse Gene 2.0 ST Array (902118). Hybridization cocktail was made using the hybridization kit (ThermoFisher Scientific, 900454) and array scans were performed according to the manufacturer's instructions using the high-resolution GeneArray Scanner 3000 7G (ThermoFisher Scientific, Affymetrix). The data were analyzed with the GeneChip Command Console Software from ThermoFisher Scientific (Affymetrix) default analysis settings.

Quantitative Proteomics and Protein Pathway Analysis. Freshly sorted LSK cells (500,000) were pelleted and flash-frozen in liquid nitrogen for shipment to the Biological Mass Spectrometry Core Facility at University of Colorado Denver. For analysis, cells were lysed with RIPA buffer (ThermoFisher) and subjected to GeLC-MS74. Excised gel pieces were destained in ammonium bicarbonate in 50% acetonitrile and dehydrated in 100% acetonitrile, trypsin digested upon reduction and alkylation of unmodified cysteine residues, and analyzed by nano-UHPLC-MS/MS (Easy-nLC1000, QExactive HF-positive ion mode (ThermoFisher)). The peptide mixture was desalted and concentrated in a Thermo Scientific Pierce C18 Tip. Samples were analyzed on an Orbitrap Fusion mass spectrometer (ThermoFisher) coupled to an Easy-nLC 1200 system (ThermoFisher) through a nano-electrospray ion source according to manufacturer's instructions. MS/MS spectra were extracted from raw data files and converted into mgf files using a Proteome Discoverer Software (ver. 2.1.0.62). The mgf files were then independently searched against the mouse database using an in-house Mascot server (Version 2.6, Matrix Science). Mass tolerances were +/−10 ppm for MS peaks, and +/−0.6 Da for MS/MS fragment ions. Trypsin specificity was used allowing for 1 missed cleavage. Met oxidation, protein N-terminal acetylation, and peptide N-terminal pyroglutamic acid formation were allowed as variable modifications while carbamidomethyl of Cys was set as a fixed modification. Scaffold (version 4.8, Proteome Software) was used to validate MS/MS based peptide and protein identifications. Peptide identifications were accepted if they could be established at greater than 95.0% probability as specified by the Peptide Prophet algorithm. Protein identifications were accepted if they could be established at greater than 99.0% probability and contained at least two identified unique peptides. Allocation of proteins to functional groups was done using the IPA software (Ingenuity Systems) and STRING database (https://string-db.org/). Protein oxidation state was performed by analyzing oxidation of methionine, carbonylation of proline to pyroglutamate and various degrees of cysteine oxidation (disulfide, glutathionylation, sulfenic, sulfonic and beta-alanine). Total protein oxidation was determined by the total number and abundance of carbonylated residues via mass spectrometry. Oxidation of methionine and carbonylation were included in this work as they showed significant differences across genotypes and interventions. To determine the acetylation state of FADS2, integrated peak areas of the FADS2 acetylated intact peptides were obtained from their extracted ion chromatograms using Qual Browser of Xcalibur 2.2 (Thermo Fisher Scientific), while b and y series ions for the MS2 of those peptides were used for the assignment of the peptide sequence and position of the acetylation 75. This approach allows identification and quantitation of both N-terminus acetylation as well as acetylation at specific amino acid residues, such as lysine (K). For the FADS2 peptides KVYNVTK and its di-acetylated form, the predicted m/z's were: 426.2529+/−0.0043 and 468.2635+/−0.0046; and for the WLVIDRK peptide and its acetylated form 465.2820+/−0.0047 and 486.2873+/−0.0049 m/z's.

Metabolomics. Freshly sorted LSK cells (100,000) were centrifuged and the pellet was flash-frozen in liquid nitrogen and shipped to the Biological Mass Spectrometry Core Facility at University of Colorado Denver. Cell pellets were lysed with lysis solution (methanol:acetonitrile:water 5:3:2 v/v/v), before ice cold extraction by vortexing for 30 minutes at 4° C. Insoluble proteins were pelleted by centrifugation (15,000 g for 10 min at 4° C.) and supernatants were analyzed using a UHPLC system (Vanquish, ThermoFisher) coupled online to a mass spectrometer (Q Exactive, ThermoFisher). Samples were resolved over a Kinetex C18 column (2.1×150 mm, 1.7 μm; Phenomenex) at 25° C. using a 9-minute gradient at 400 μl/min from 5% to 95% B (A: water/0.1% formic acid; B: acetonitrile/0.1% formic acid). MS analysis and data analysis were performed as described before. Metabolite assignments were performed using a metabolomics data analyzer (MAVEN). Metabolic pathway analysis was performed using the MetaboAnalyst software.

Measurement of intracellular protein degradation. Freshly isolated LSK cells (25,000 cells) were labeled with [³H]Leucine (2 μCi/ml) in stem span medium containing 100nmol mSCF for 24 hours in a 48-well plate pre-coated with CELL-TAK. After extensive washing, media (with 2.8 mM unlabeled leucine) was added and cells were incubated at 37° C. Where indicated, 20 mM NH4C1 and 1001.IM Leupeptin (Sigma) were added to the media. Aliquots of the medium taken at 12 hours were precipitated with trichloroacetic acid and proteolysis was calculated as the percentage of initial total acid precipitable radioactivity (protein) transformed to acid soluble (peptides and amino acids) at each time point. Lysosomal proteolysis was determined as percentage of proteolysis sensitive to the combination of lysosomal inhibitors.

Other procedures. For quantitative PCR, RNA was extracted using RNeasy Micro kit (Qiagen) according to the manufacturer's protocols. Transcripts were reverse transcribed according to manufacturer protocols (Invitrogen), and qPCR was performed using SYBR Green (Applied Biosystems). The following primers were used: mouse FADS2, F-5′-gctctcagatcaccgaggac-3′, R-5′-agtgccgaagtacgagagga-3′. Immunoblot for different tissues (100 μg total protein) was performed in nitrocellulose membranes after tissue sonication in RIPA buffer. The pCMV6-Entry-FADS2-Myc-DDK plasmid was purchased from Origene (MR207091), single point mutation of FADS2 42K to Q and A was performed at GenScript.

Statistics, sample size and software. All data are presented both as individual values (symbols) and mean +standard error of the mean (sem). Unpaired t-test, two-way ANOVA test, one-way ANOVA tests, chi-square test, log-rank (Mantel-Cox) test were used for the statistics as indicated in each figure legend. In all instances, “n” refers to individual experiments or animals and is indicated in the figure legends. The number of animals used per experiment was calculated through power analysis based in previous results. Animals were randomly attributed to control or treatment groups after separating them according to genotype. No mouse was excluded from the analysis unless there were technical reasons, or the mouse was determined to be in very poor health by the veterinarian. Outliers were determined by the ROUT method (Q=1%). Investigators were blinded to the treatment during data collection and analysis and unblinding was done when the analysis was completed for plotting. All parameters were analyzed using independent sample tests and were tested for normal distribution using Shapiro-Wilk normality test. For software, image analysis was performed using Image J (NIH), pathway analysis using the Ingenuity Pathway Analysis (Ingenuity Systems) and STRING database, HSC frequency within the tested cell population was estimated using ELDA software, all graphical plots were made using GraphPad Prism software 7.04 (GraphPad) and images were assembled with Adobe Photoshop CC software (Adobe).

Measurement of CMA Activity in vitro. Suitability of compounds for use in the disclosed methods as a CMA Activator can be determined by measurement of In vitro CMA activity. The photoactivatable CMA reporter assay was constructed by inserting a sequence of 21 amino acid of Ribonuclease A bearing the CMA- targeting motif in the N-terminus multicloning site of the photoactivatable protein mCherryl or the photoswitchable protein.

NIH 3T3 fibroblasts were stably transduced with a photoconvertible CMA reporter, KFERQ-Dendra and were photoswitched by exposure to a 3.5 MA (constant current) LED (Norlux, 405 nm) for 10 minutes and at the desired times fixed in 3% formaldehyde. Test cells are exposed to the indicated concentrations of the compounds, e.g. for 12 hours or 24 hours.

Cells are imaged, e.g., by using high content microscopy (Operetta, Perkin Elmer) or by capturing images with an Axiovert 200 fluorescence microscope (Zeiss) with apotome and equipped with a 63×1.4 NA oil objective lens and red (ex. 570/30 nm, em. 615/30 nm), cyan (ex. 365/50 nm and em. 530/45 nm) and green (ex. 475/40 nm and em. 535/45 nm) filter sets (Chroma). Images were acquired with a high-resolution CCD camera after optical sectioning through the apotome. CMA activity is measured as the average number of fluorescent puncta (CMA active lysosomes) per cell. Values are expressed relative to values in untreated cells that were assigned an arbitrary value of 1 and are mean of >2,500 cells counted per condition. The S.D. in all instances was <0.01% mean value.

### Example 2. CMA is Upregulated in Activated HSC

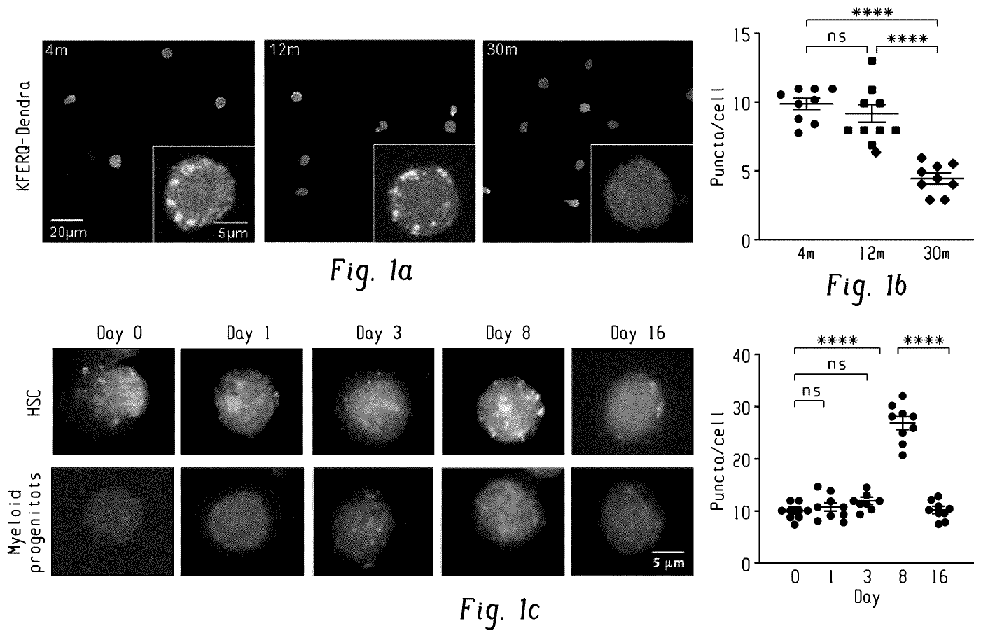

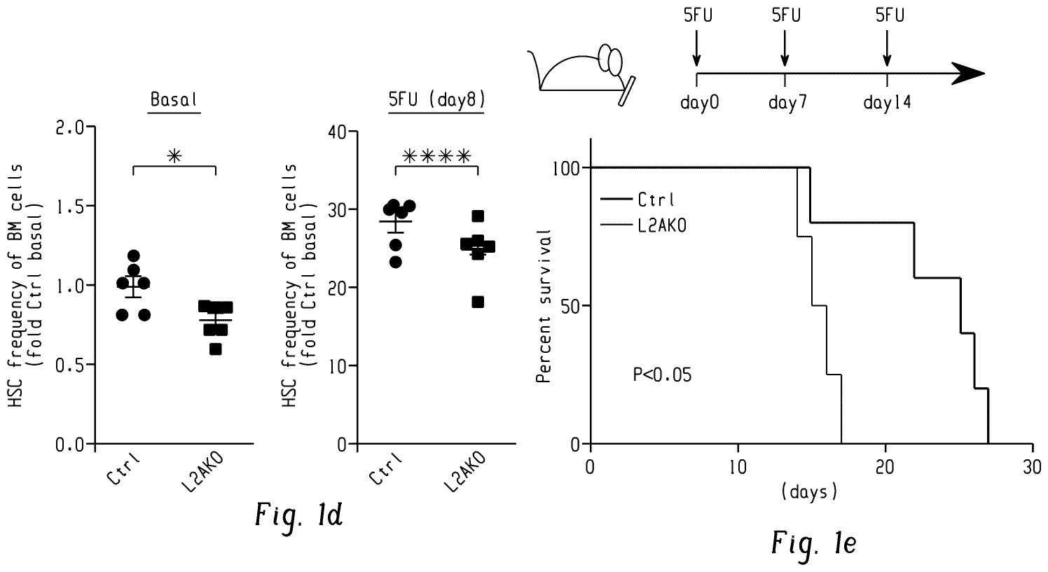

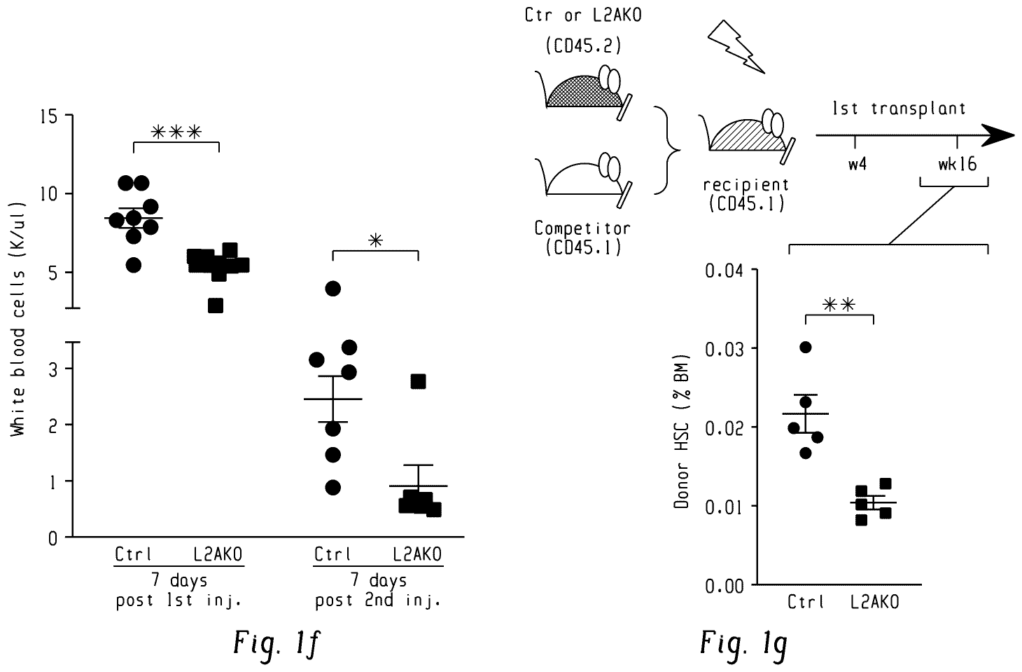

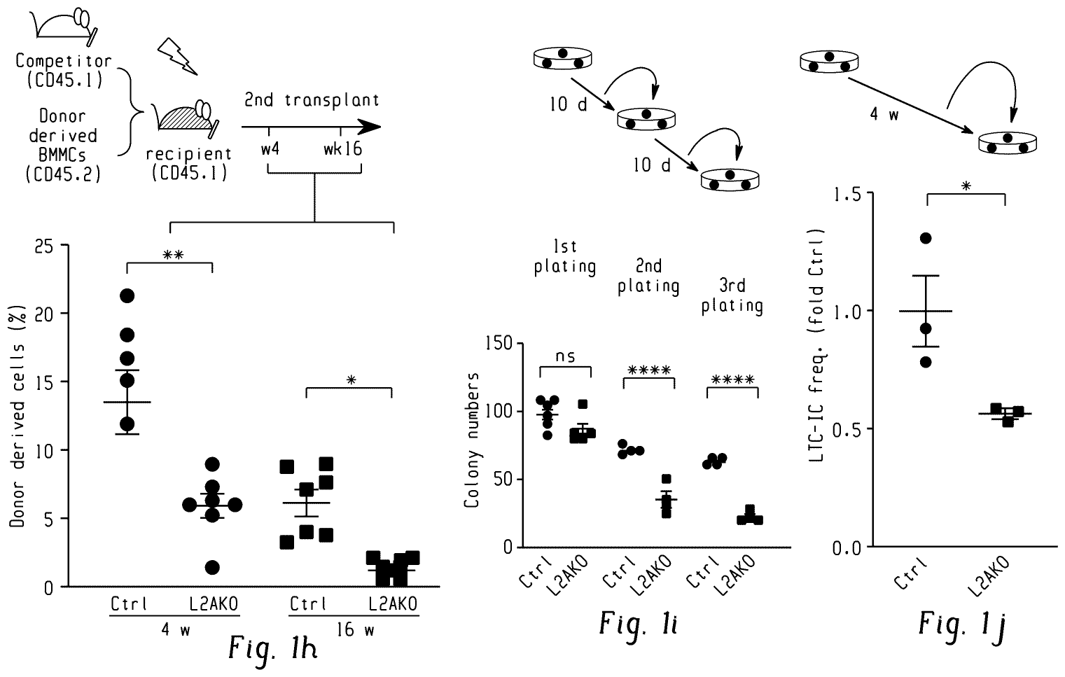

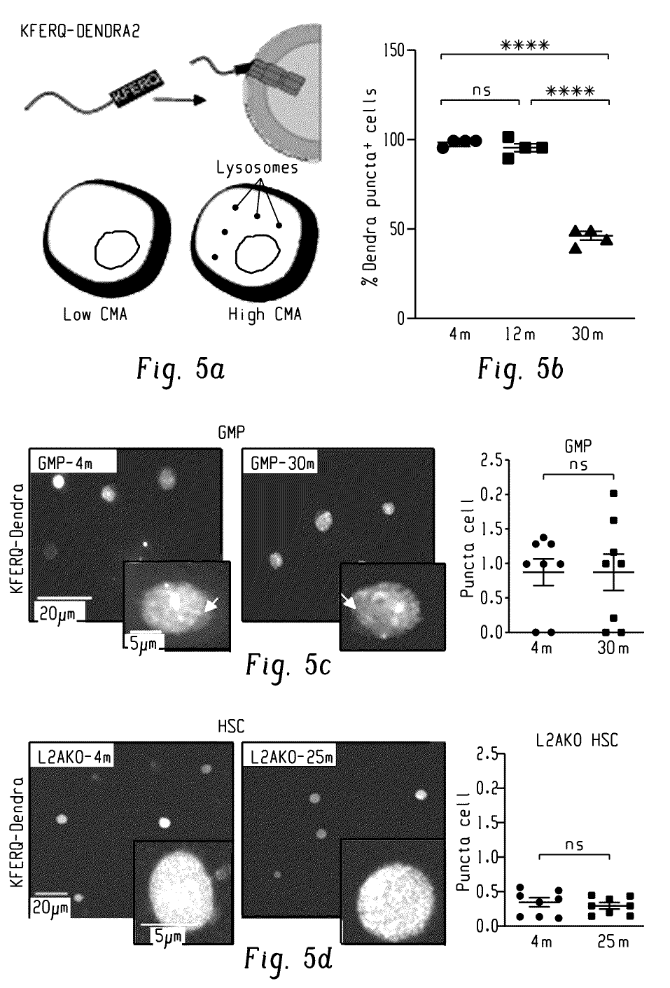

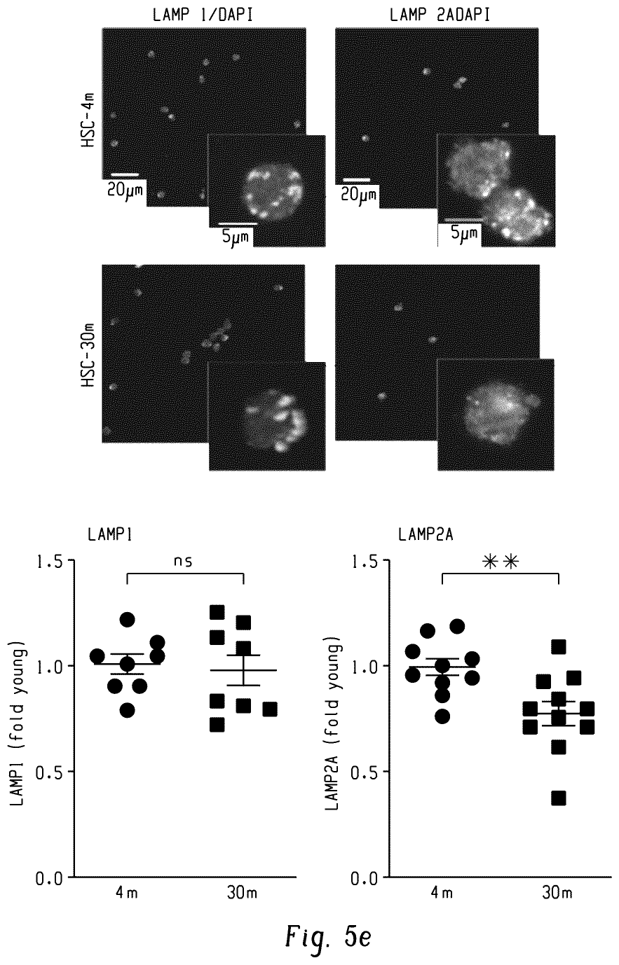

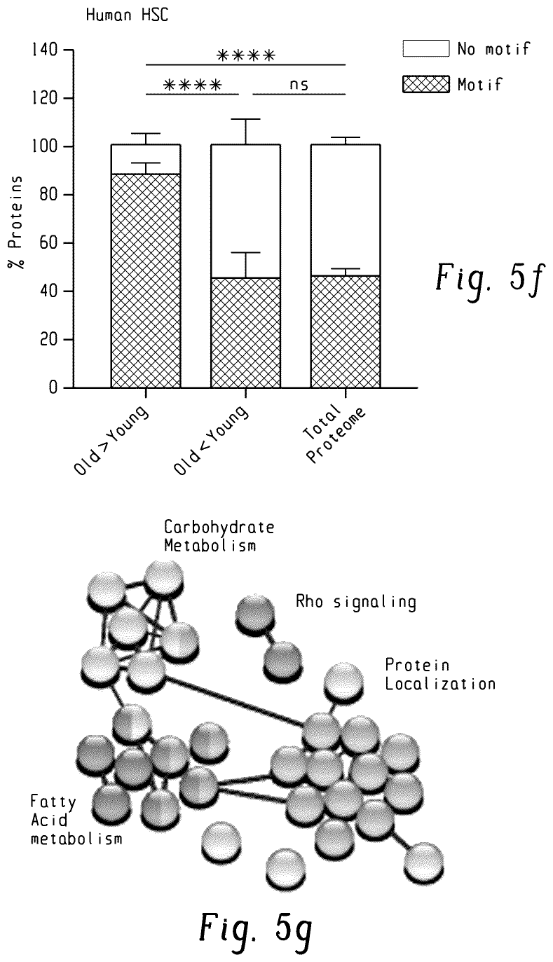

Using transgenic mice expressing a CMA reporter (KFERQ-Dendra2) that highlights lysosomes as fluorescent puncta when CMA is activated (FIG. 5a) we found reduced CMA activity in HSC isolated from the older age mice compared to young mice. Both, CMA activity per cell and fraction of HSCs active for CMA decreased with age, whereas CMA remained unchanged with age in granulocyte-monocyte progenitor (GMP) cells (FIG. 1a,b and FIG. 5 b,c). Dendra fluorescent puncta were not detected in CMA-incompetent mice (knock-out for LAMP-2A (L2A), L2AKO) at any age, confirming the specificity of the CMA reporter (FIG. 5d). Reduced CMA in old mice HSC is not a consequence of overall lower lysosome abundance with age, as levels of the endo lysosomal marker LAMP1 were comparable in young and old mice HSC. In contrast, levels of L2A, the spliced variant of the lamp2 gene required for CMA, were significantly reduced in HSCs from old mice, further supporting impairment of CMA activity in HSC with age (FIG. 5e). A similar decline of CMA with age may also occur in humans, as we found significantly higher abundance of proteins bearing CMA-targeting motifs among the proteins reported to accumulate with age in human HSC (FIG. 5f).

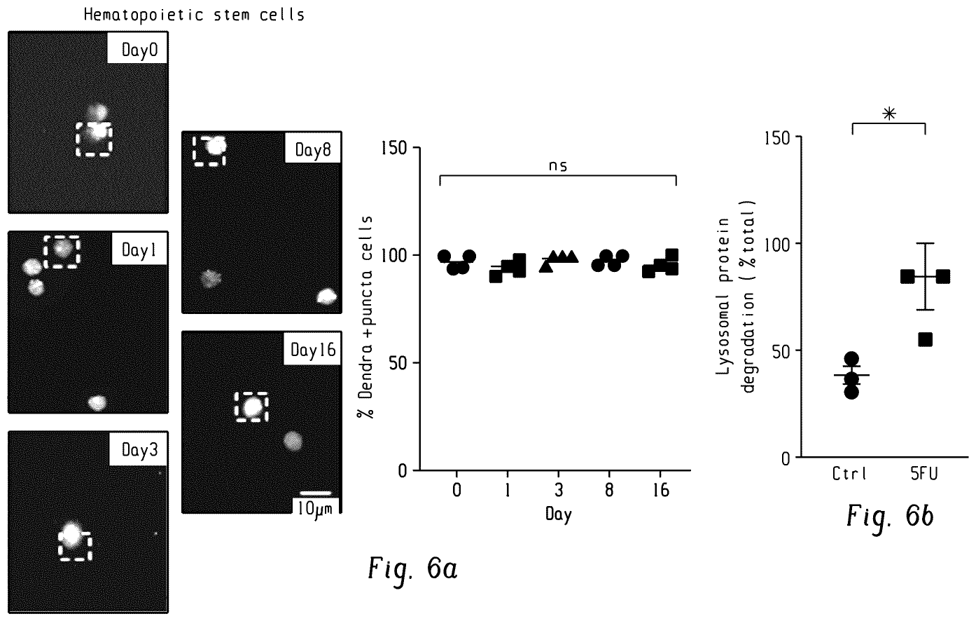

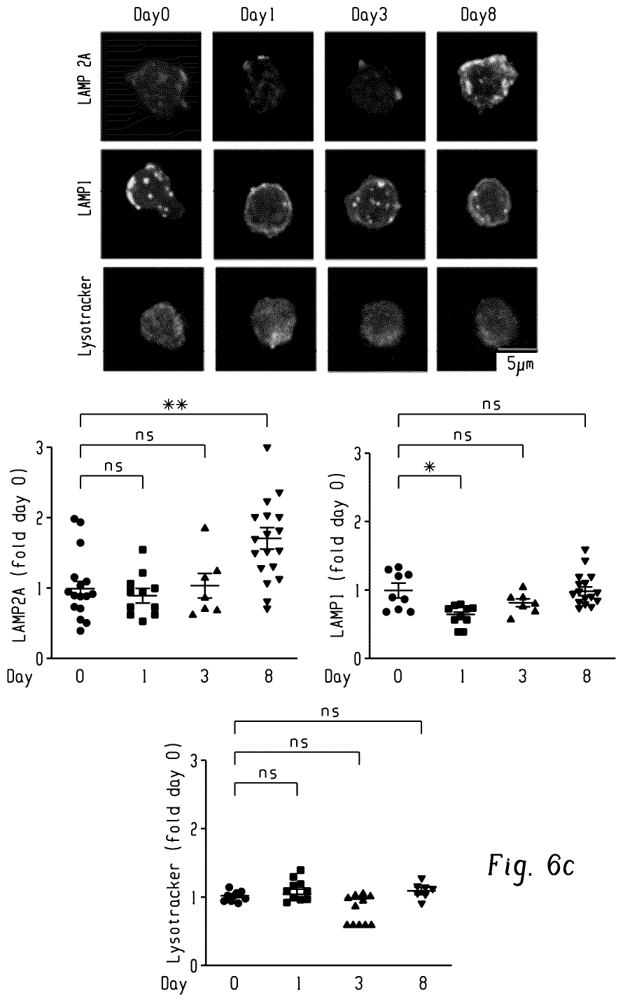

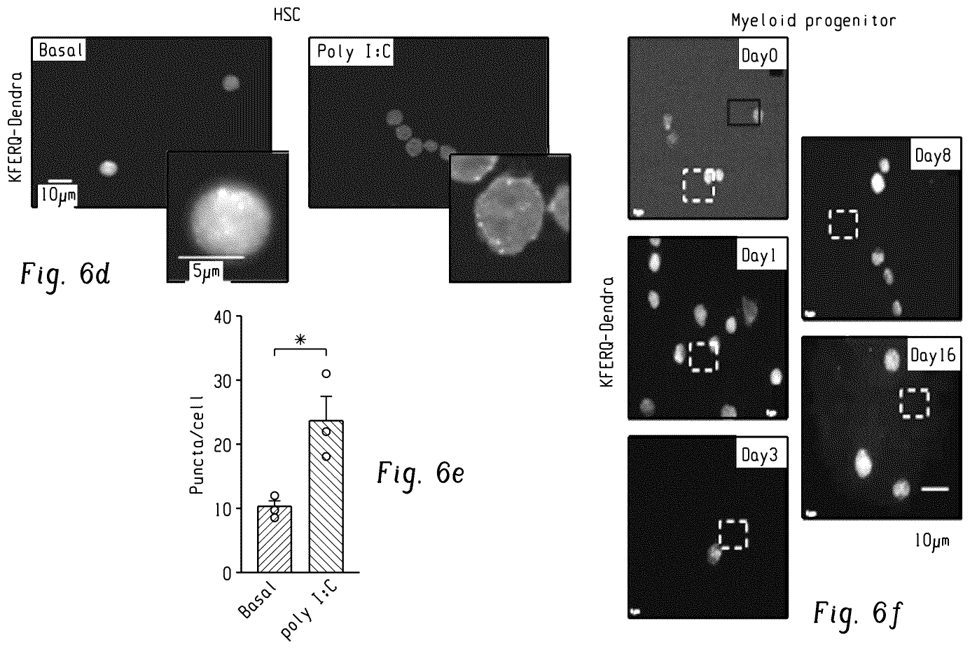

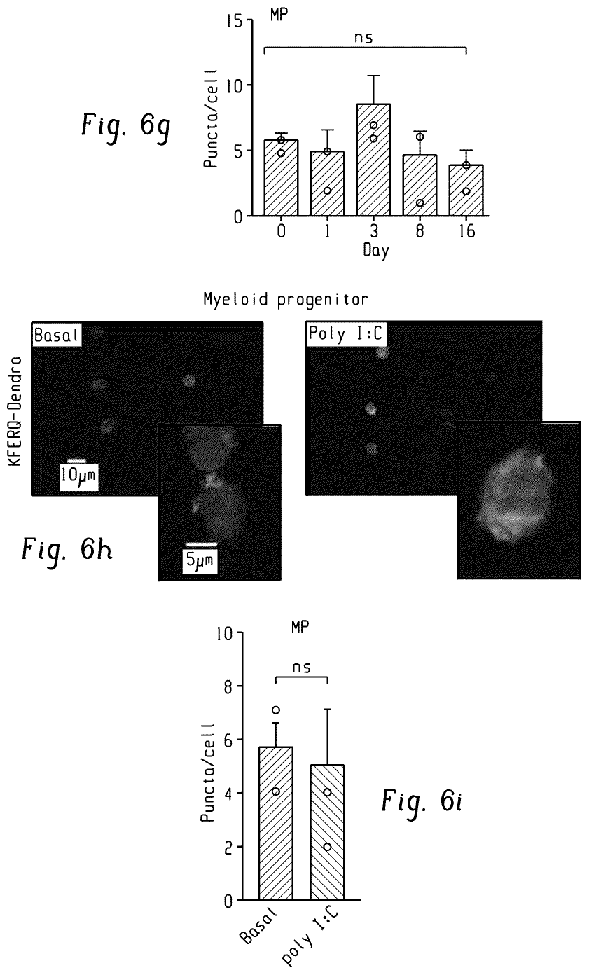

To test if declining CMA contributes to aging-associated functional alterations of HSC, and because the role of CMA in hematopoietic stem cells has not been studied before, we first measured CMA activity in steady-state quiescent and activated HSC isolated from KFERQ-Dendra mice. Quiescent HSC showed higher basal CMA activity than myeloid progenitors, and further upregulate CMA upon in vivo activation of HSC following exposure to myeloablative agent 5-fluorouracil (5FU) (FIG. 5c and FIG. 6a). Maximal CMA upregulation was observed by day 8 after 5FU injection, with CMA returning to basal levels by day 16 (FIG. 1c). Increase in CMA was mainly due to higher activity per cell as the fraction of HSC active for CMA remained unchanged (FIG. 6a, right). We confirmed that lysosomal degradation of intracellular proteins and levels of LAMP2A, but not of other lysosomal markers, were also significantly higher at day 8 post activation (FIG. 6b,c). In vivo treatment with polyI:C, which potently induces HSC cell cycle entry30, also upregulated CMA in these cells (FIG. 6d, e), whereas none of these interventions resulted in changes in CMA activity in myeloid progenitor cells (FIG. 6f-i). We conclude that HSC upregulate CMA upon activation.

### Example 3. CMA Prevents HSC Depletion Upon Activation

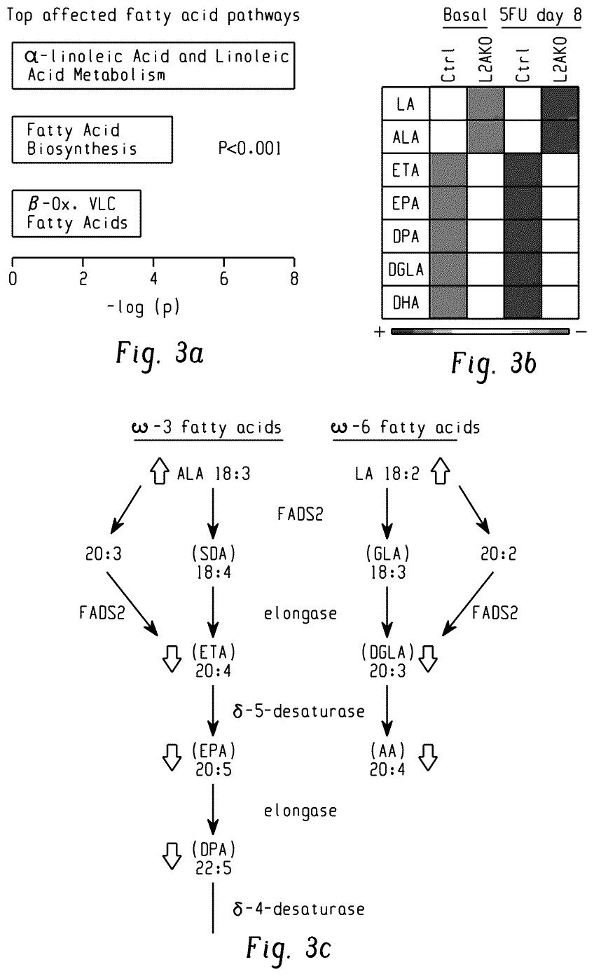

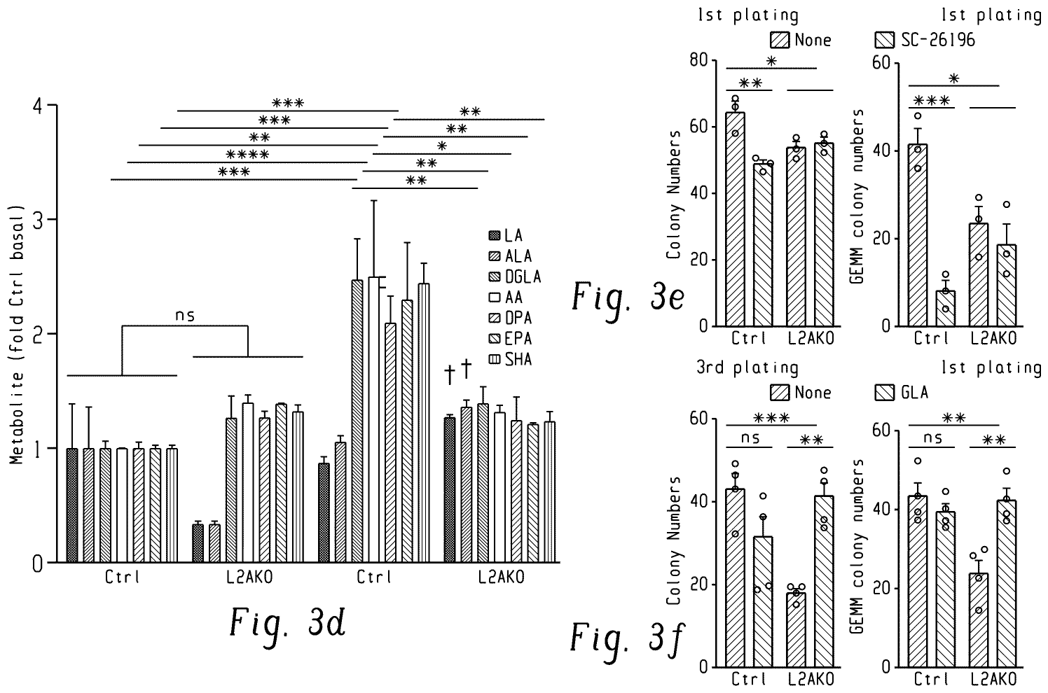

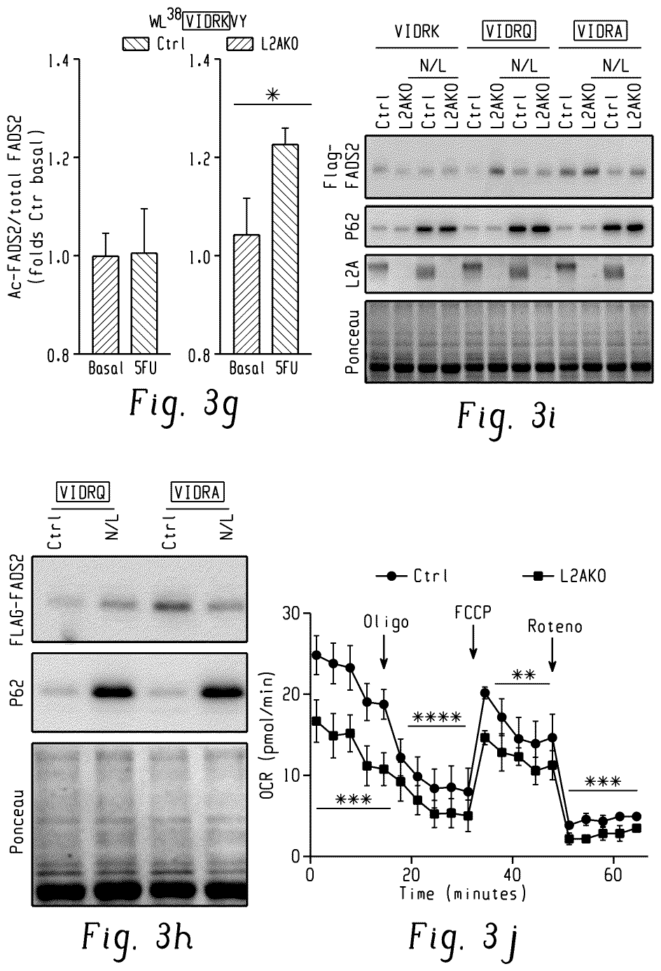

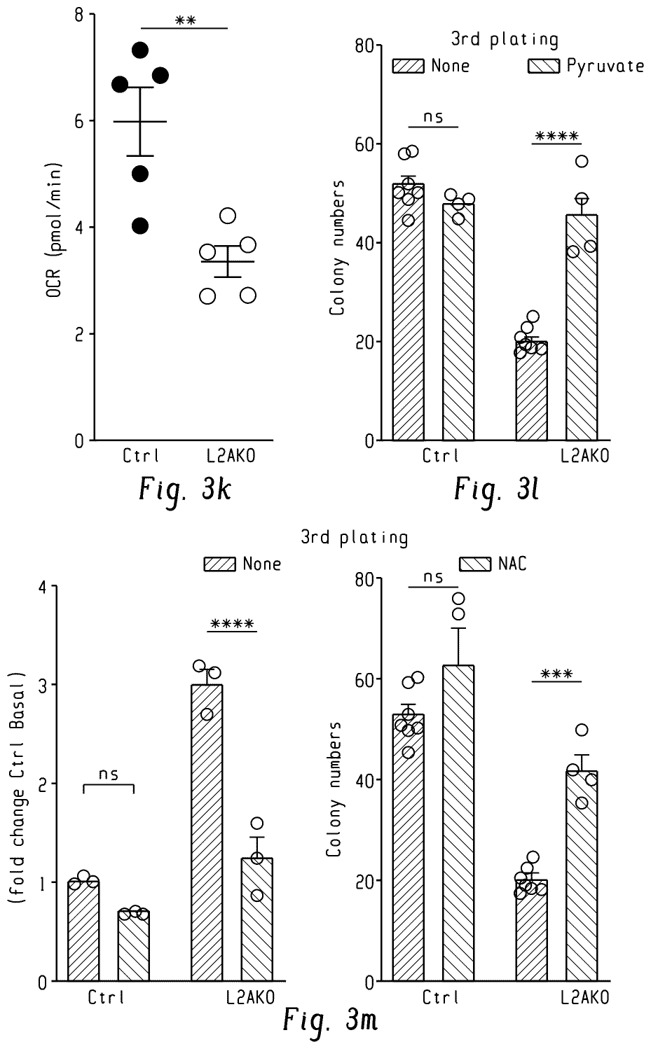

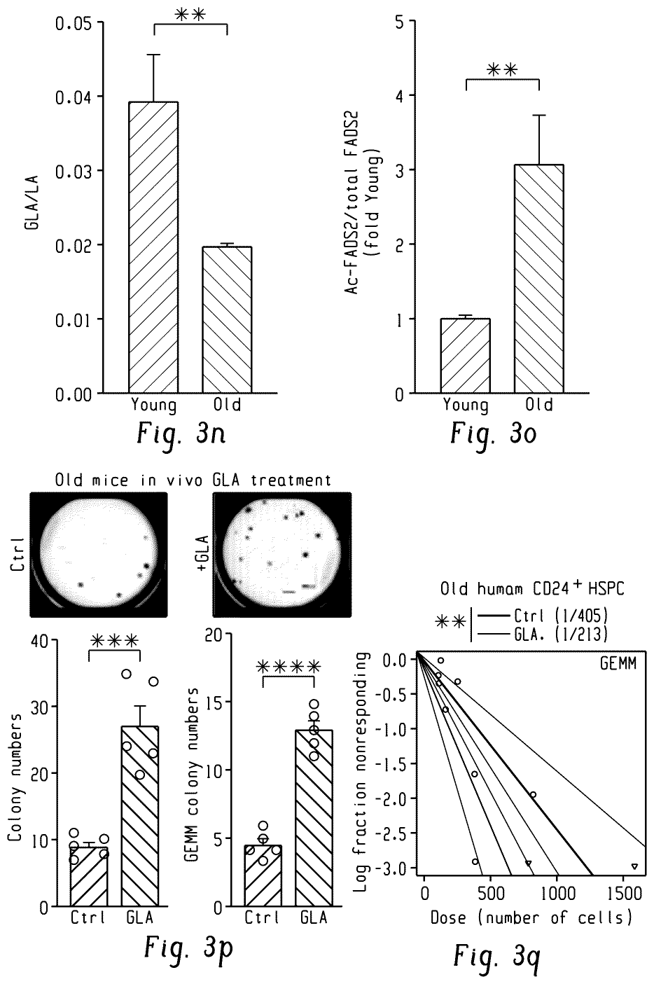

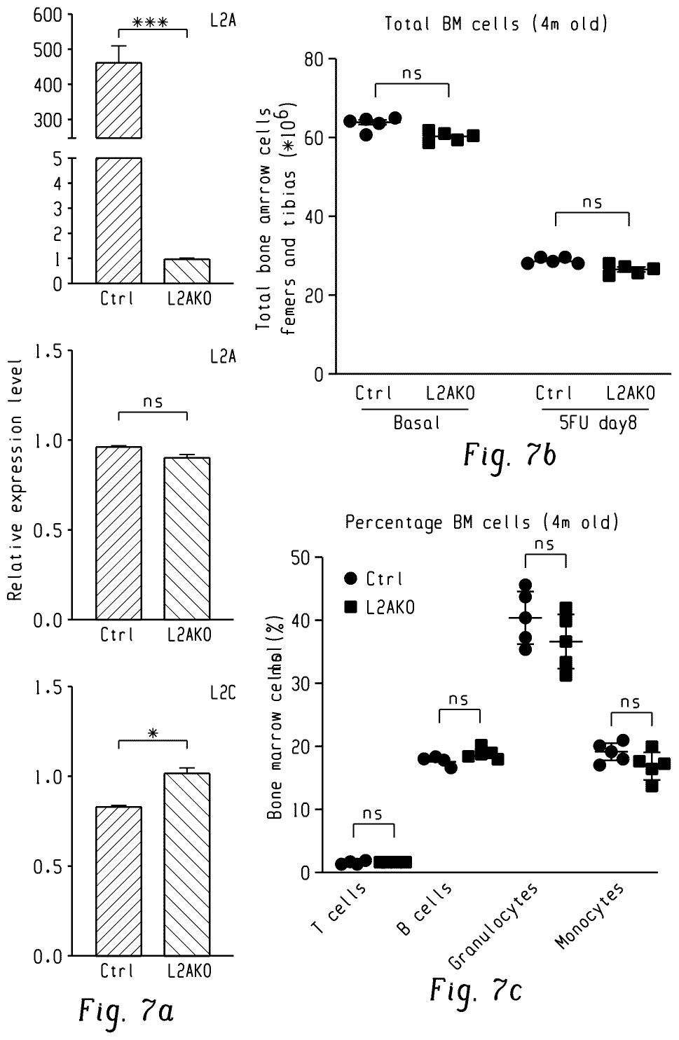

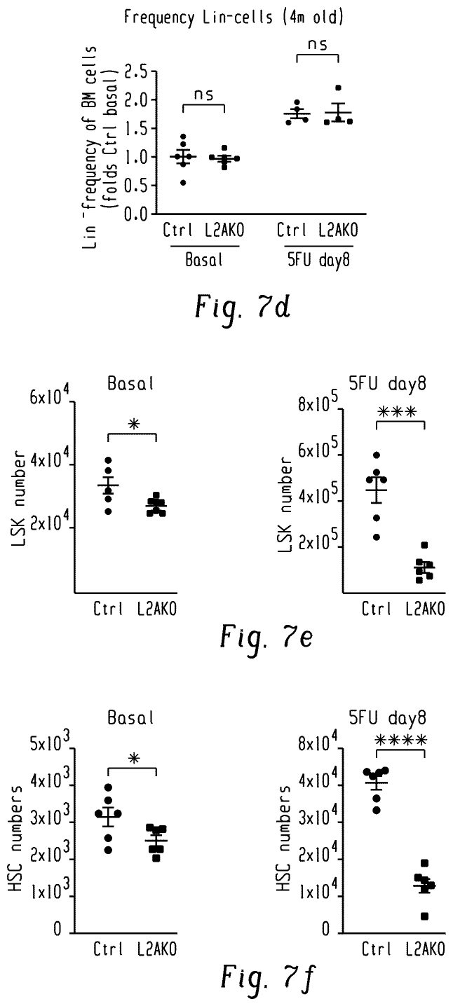

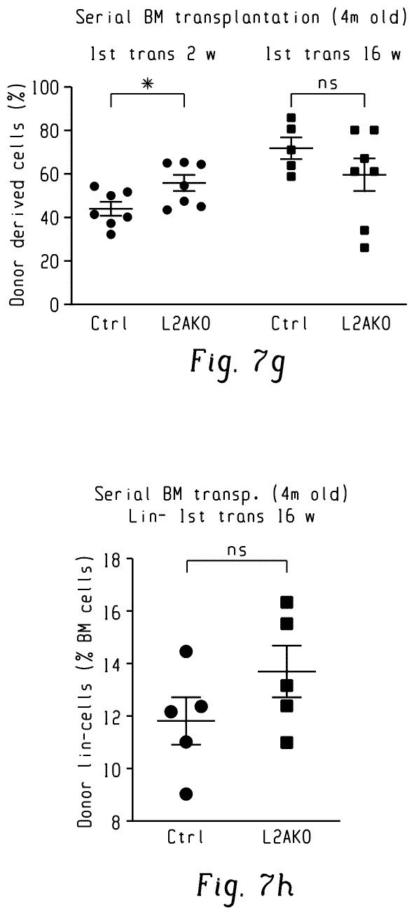

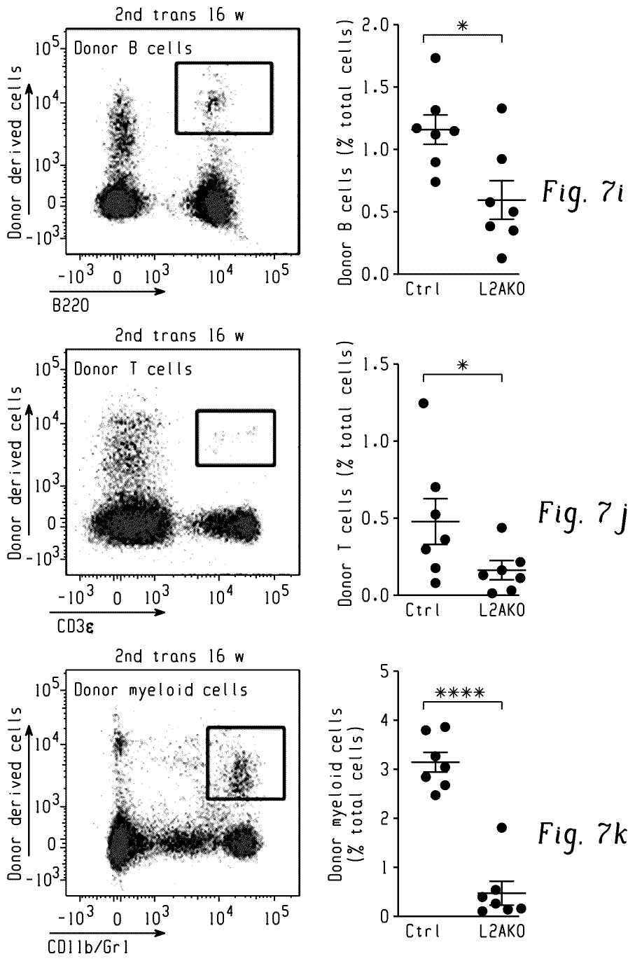

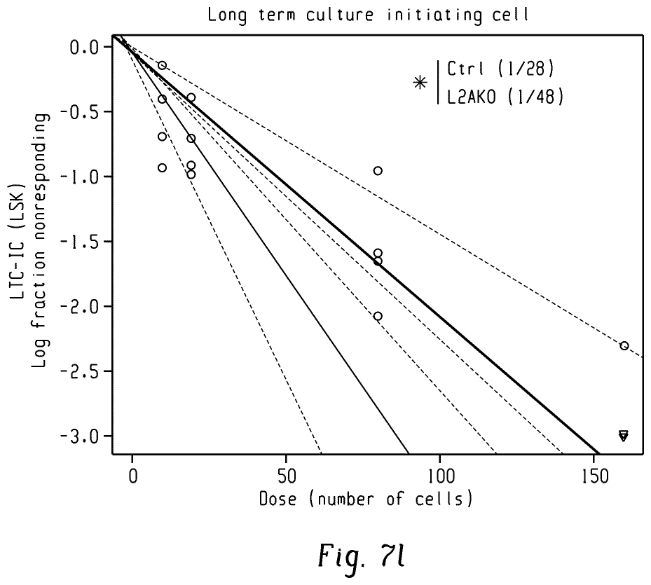

To determine the functional relevance of CMA upregulation upon HSC activation, we selectively eliminated CMA in hematopoietic cells by crossing LAMP2Af/f mice with Vav-iCre mice (Vav-iCre-LAMP2Af/f mice, hereafter termed L2AKO). L2AKO mice allow for hematopoietic cell-specific deletion of LAMP2A while preserving expression of the other Lamp2 splice variants (FIG. 3a). Young L2AKO mice did not display differences in the total number of hematopoietic cells in the bone marrow (BM) or in the frequencies of T (CD3_{ε+}), B (B220+), mature myeloid (CD11b+Gr1+) or of lineage-negative (Lin-) progenitor cell populations when compared to litter mate controls (FIG. 3b-d), demonstrating that loss of CMA is compatible with multilineage hematopoiesis. However, we observed a moderate but significant decrease in the frequency and number of stem cell-enriched (Lin−) Sca-1+cKit+, LSK) and rigorously defined phenotypical HSC (Lin−Sca-1+cKit+CD150+CD48—) populations at steady-state, which became markedly more pronounced following 5FU-mediated stem cell activation (FIG. 1d and FIG. 7e,f). In fact, the expected expansion of the HSC pool, a prerequisite for restoring mature hematopoietic cell populations following 5FU-mediated myeloablation, was also significantly attenuated in L2AKO mice (approximately 2.5-fold less expansion than control cells) (FIG. 1d and FIG. 7f). The reduction in the steady-state HSC pool size and regeneration capabilities in L2AKO mice was attributable to a specific inhibition of the HSC compartment, as we did not find detectable changes in total BM cellularity in these mice (FIG. 7b).

Constitutive acute HSC activation using serial 5FU injections revealed that L2AKO mice could not recover their white blood cell counts and died of pre-mature bone marrow failure (FIG. 1e,f). Furthermore, serial transplantation and competitive BM repopulation, a more chronic paradigm of HSC activation (FIG. 1g), showed that CMA-deficient HSC were equally capable to engraft and reconstitute lethally irradiated congenic recipients upon primary transplantation but that, 16 weeks later, the number of L2AKO donor-derived HSC was reduced by 50%, while the total amount of immature Lin- cells was unaltered (FIG. 1h and FIG. 7g,h). Secondary competitive bone marrow transplantation revealed a persistently reduced reconstitution with donor cells in all blood lineages in recipients of CMA-deficient cells compared to controls demonstrating a loss of functional stem cells (FIG. 1i and FIG. 7i-k). Ex vivo studies also showed impaired self-renewal of L2AKO HSC (FIG. 1j,k and FIG. 7l), in further support of a cell intrinsic functional defect of CMA-deficient HSC.

Together, these findings demonstrate that albeit CMA is dispensable for multilineage blood cell differentiation, it is necessary to maintain functional HSC during steady state and, particularly, upon activation.

### Example 4. Cma Deficiency Impairs HSC Activation

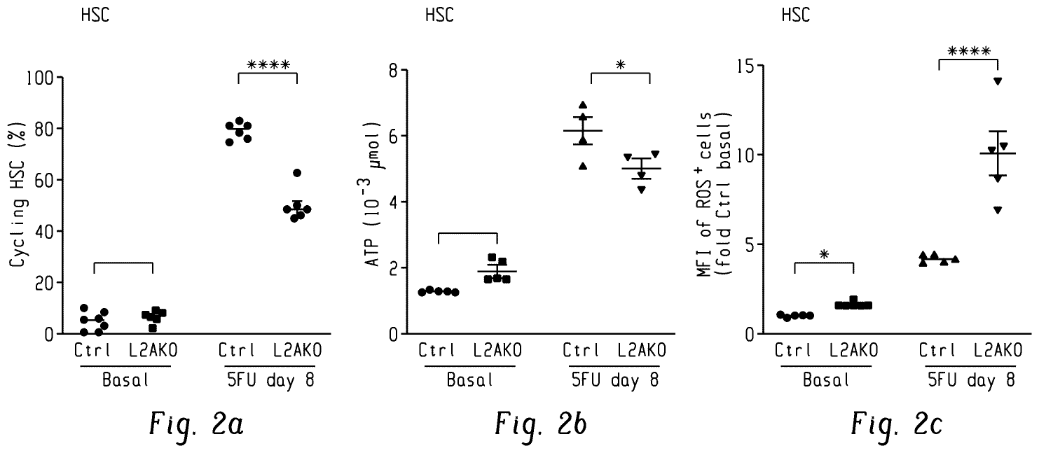

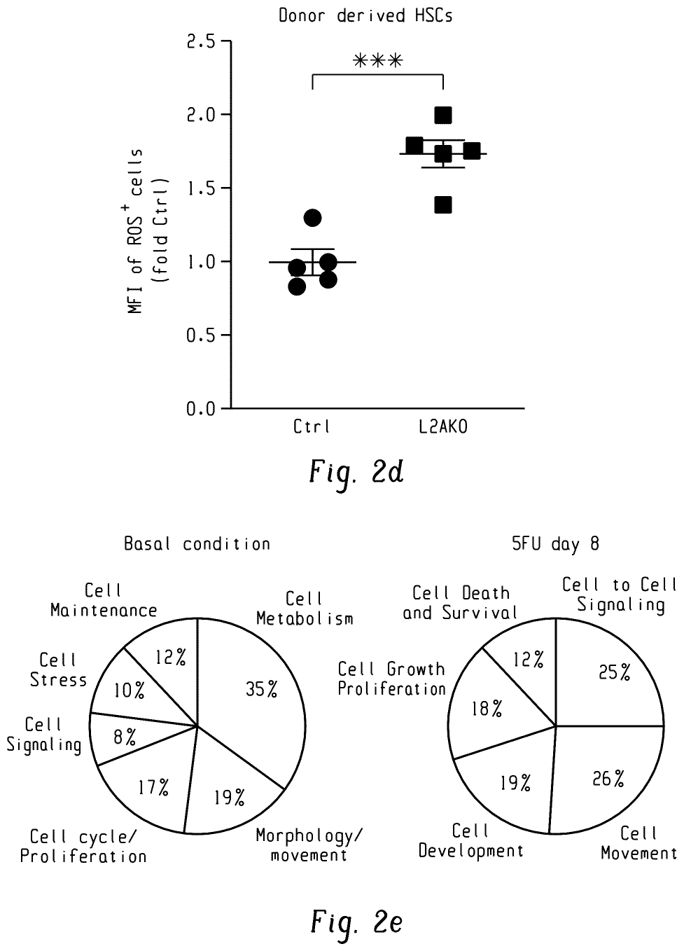

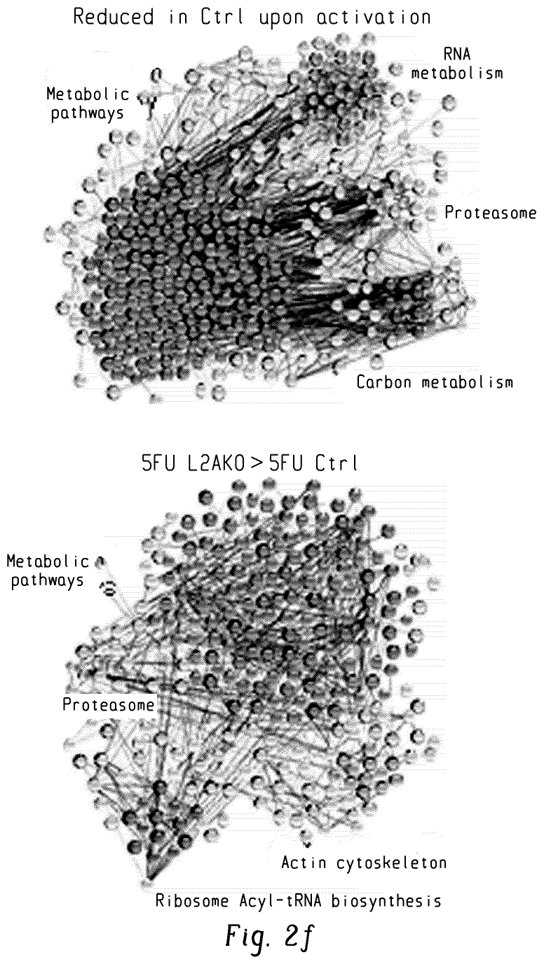

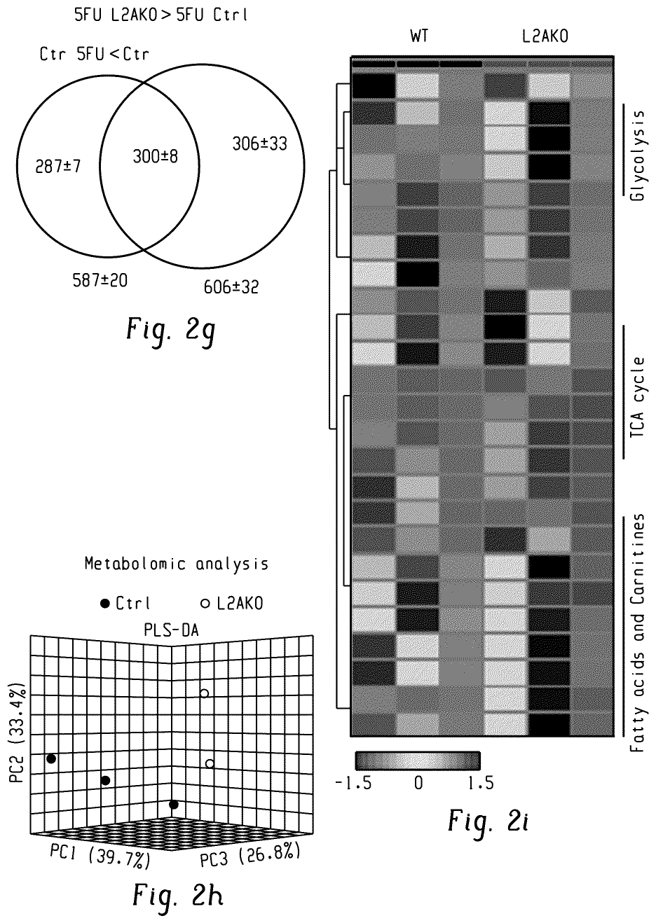

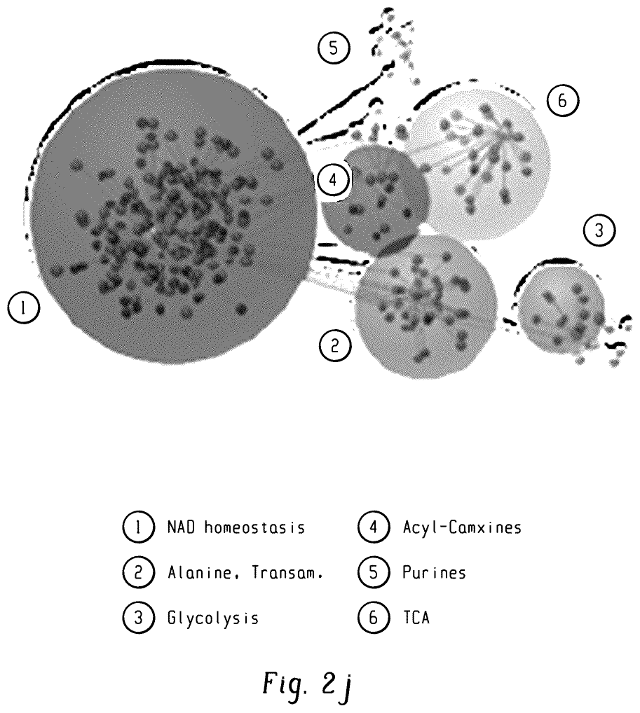

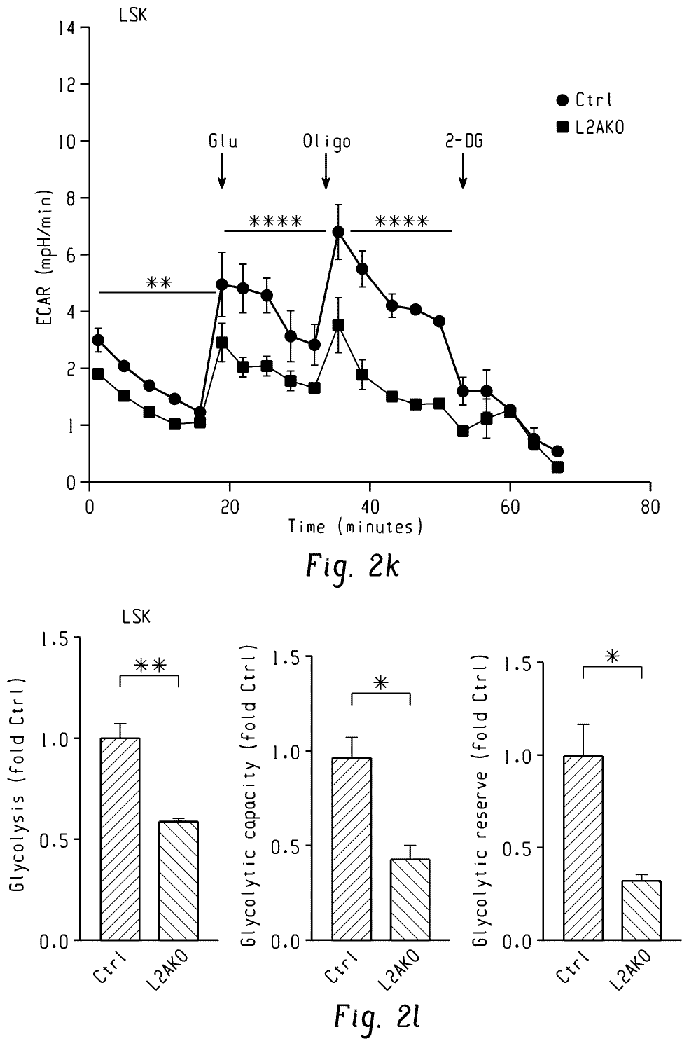

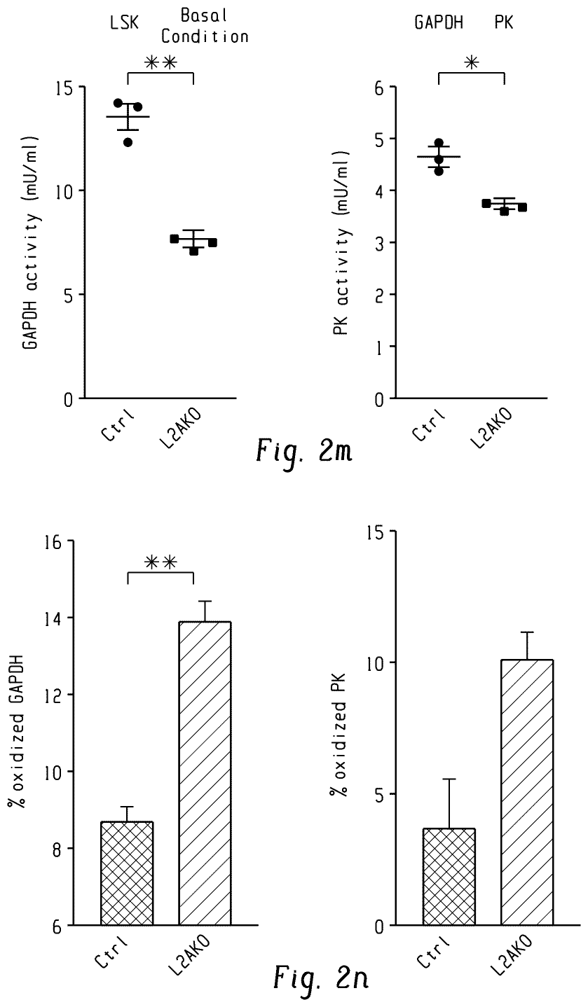

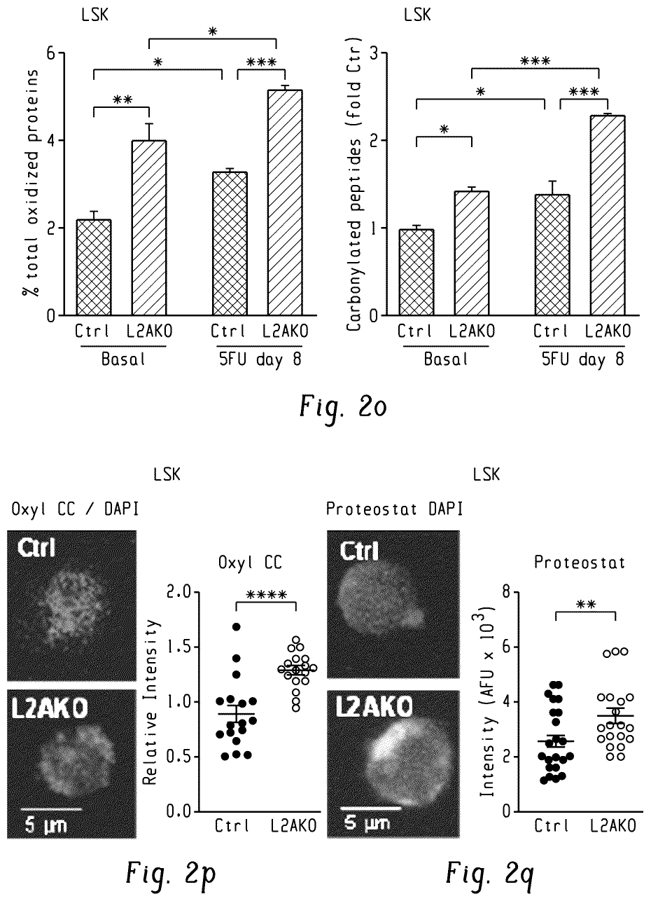

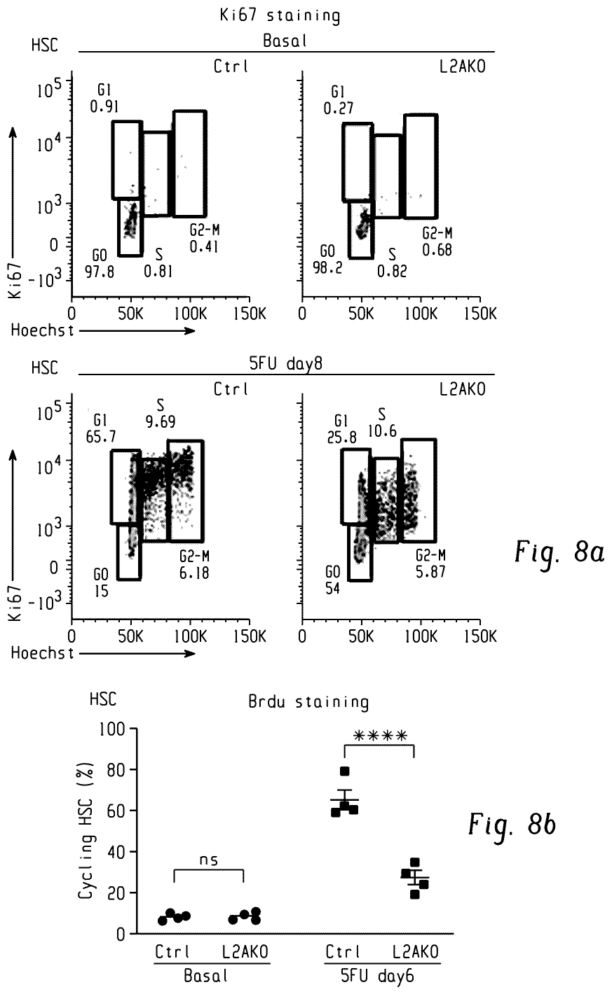

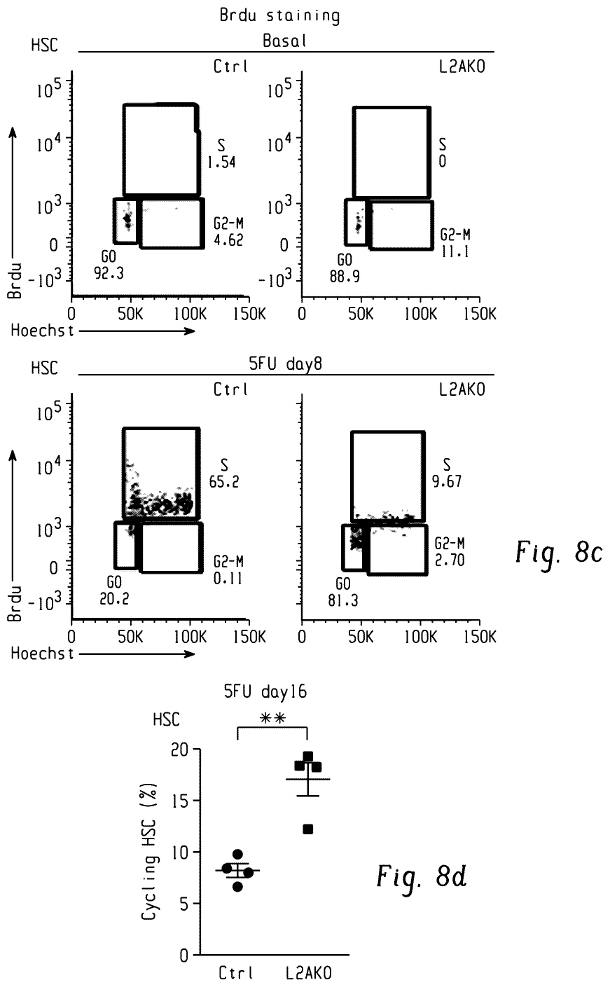

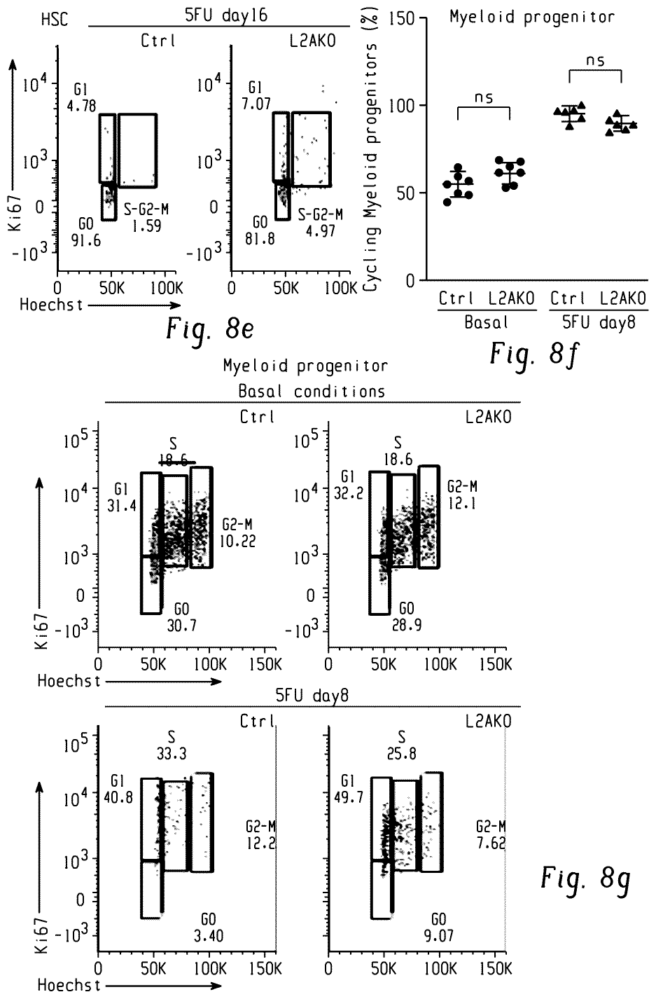

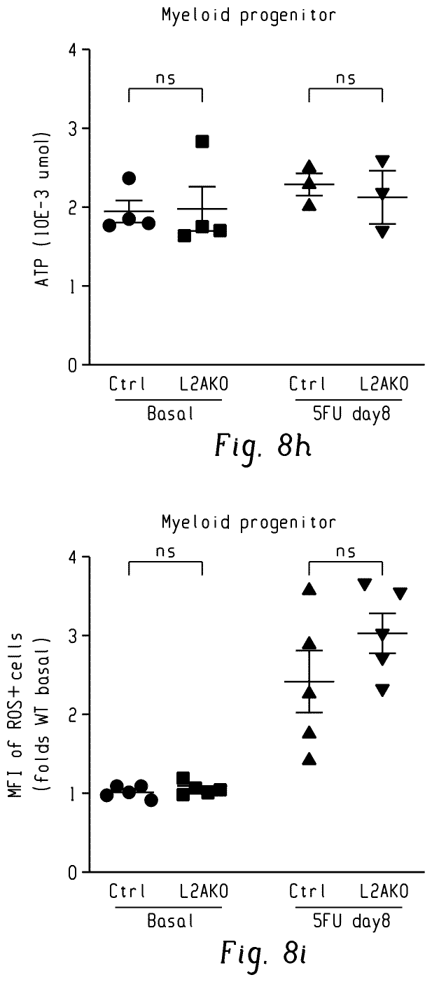

The reduced BM repopulation capability of CMA-deficient HSC originates, at least in part, from a marked reduction in the pool of actively cycling HSC 8 days after 5FU injection, as more than 50% of L2AKO HSC (vs. 15% of control HSC) were found in G0 at that time (FIG. 2a and FIG. 8a-c). Interestingly, a higher fraction of L2AKO HSC was still cycling when most control HSC had fully returned to quiescence (FIG. 8d,e). We did not find differences in cell cycle status between control and L2AKO myeloid progenitor cells (FIG. 8f,g), supporting that CMA is important specifically for HSC cycling.

L2AKO HSC, but not myeloid progenitor cells, showed a reduced ability to increase intracellular ATP content and a more pronounced increase in ROS levels in response to 5FU when compared to control cells (FIG. 2b,c and FIG. 8h,i). Higher ROS levels—a common manifestation of metabolic deregulation and proven cause of impaired HSC self-renewal ability were also observed in L2AKO bone marrow derived HSC upon transplantation (FIG. 2d). Overall, our data support that CMA is required to promote timely HSC cell cycle entry upon activation and that the poor energetic status and elevated ROS content of CMA-deficient HSC may be behind their compromised self-renewal capacity.

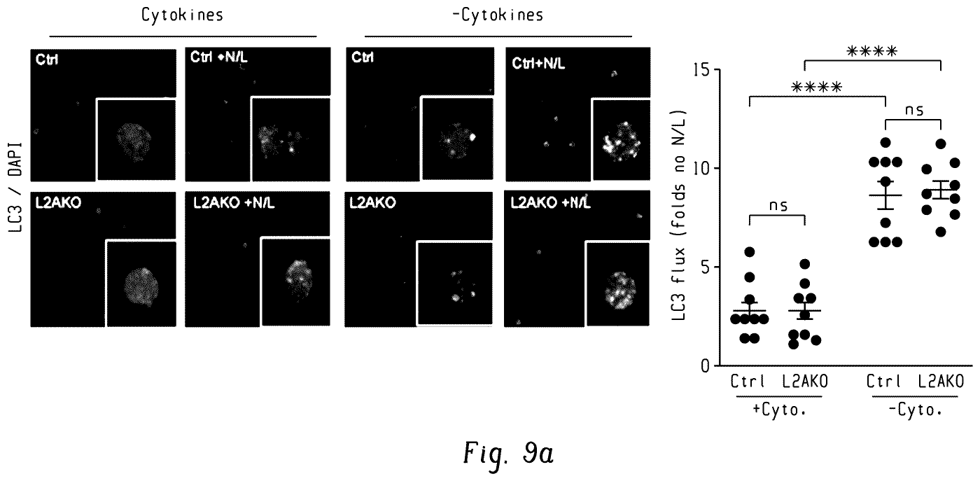

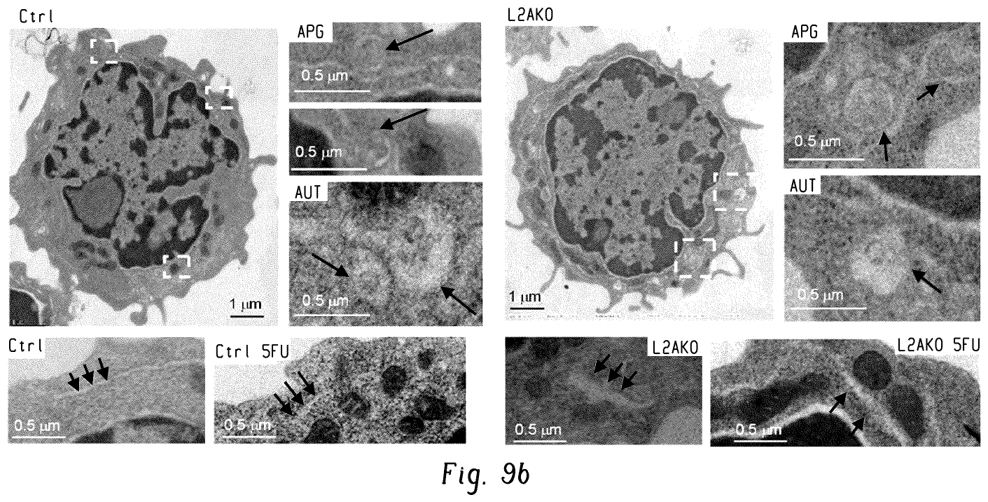

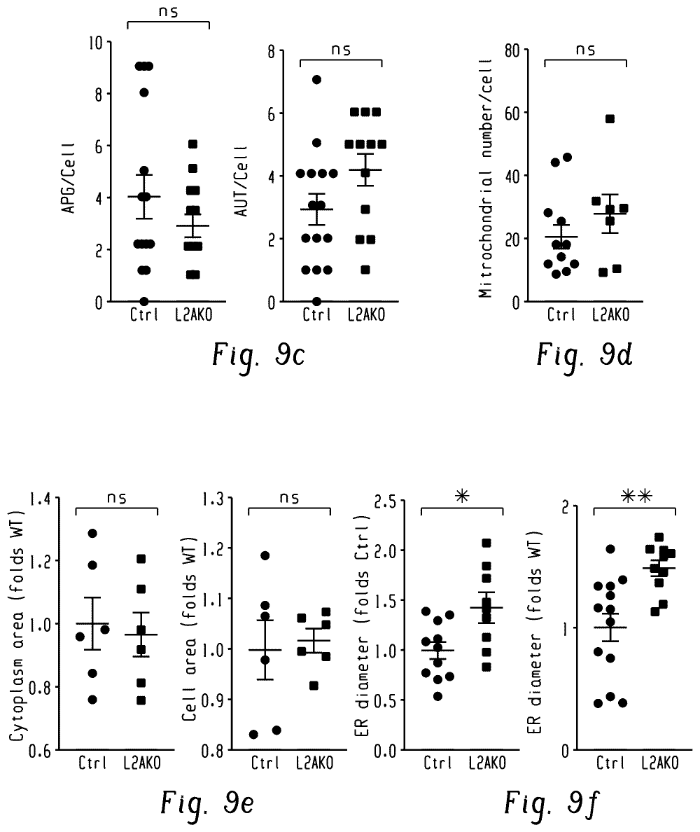

Although blockage of macroautophagy in HSC also increases intracellular ROS, the phenotype of CMA-deficient HSC was not secondary to changes in the status of macroautophagy, which was comparable between control and L2AKO HSC in both basal and cytokine starved conditions (FIG. 9a-d show no differences in LC3 flux, the autophagy and lysosomal TFEB-dependent transcriptional program or in the number of autophagic compartments between both cell genotypes). Furthermore, blockage of macroautophagy in HSC increases proliferation rates and causes pronounced mitochondrial alterations, which is in clear contrast with the reduced cycling and absence of noticeable changes in mitochondrial morphology and cellular and cytosolic areas of CMA-deficient HSC (FIG. 9e,f).

### Example 5. CMA-Dependent Proteome Remodeling in HSC

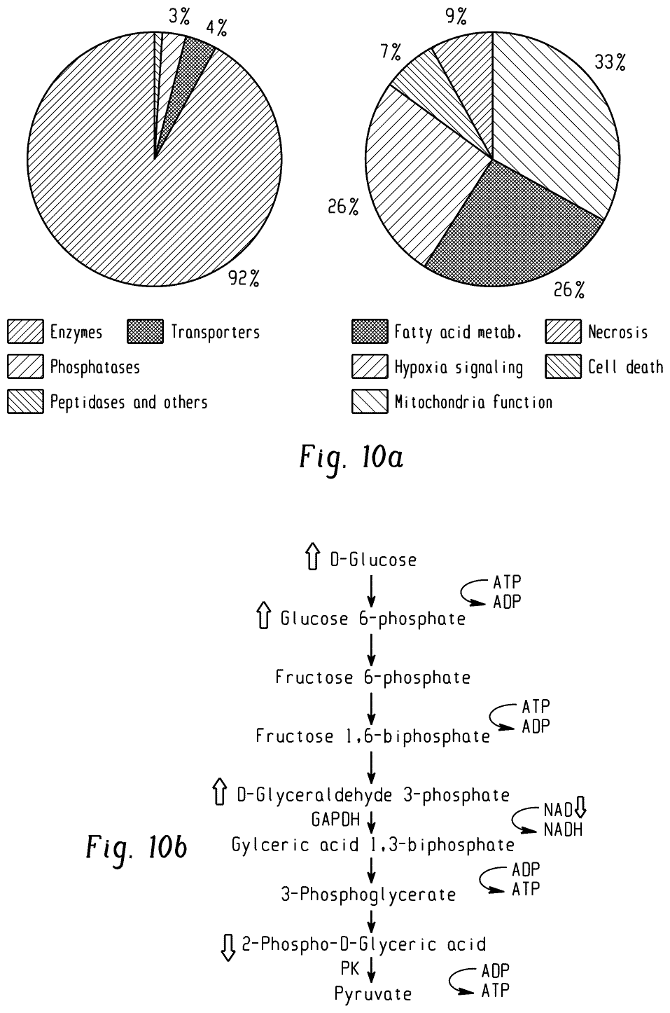

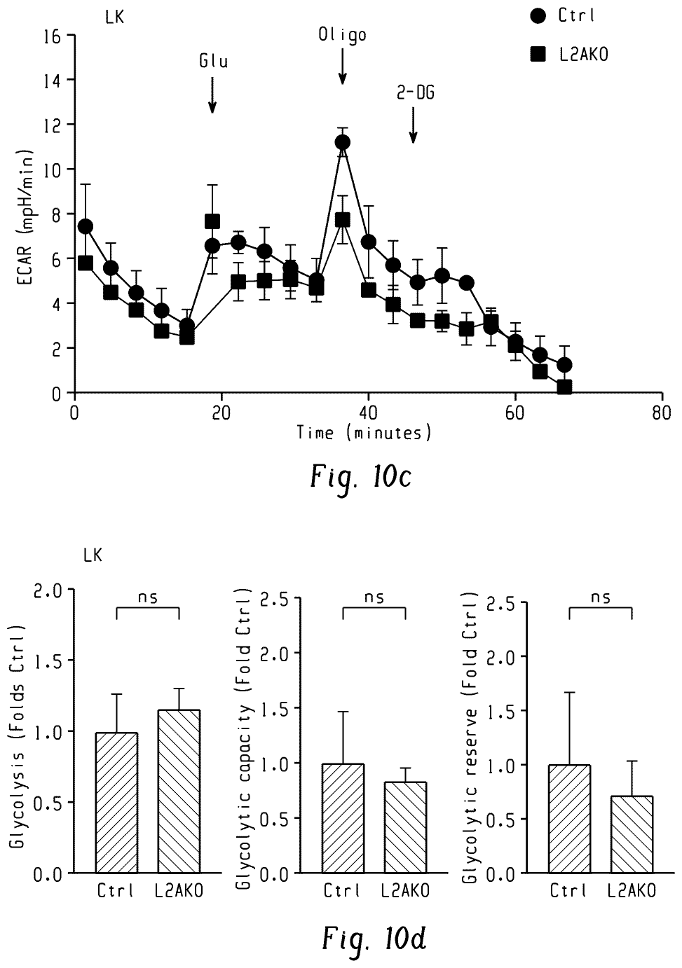

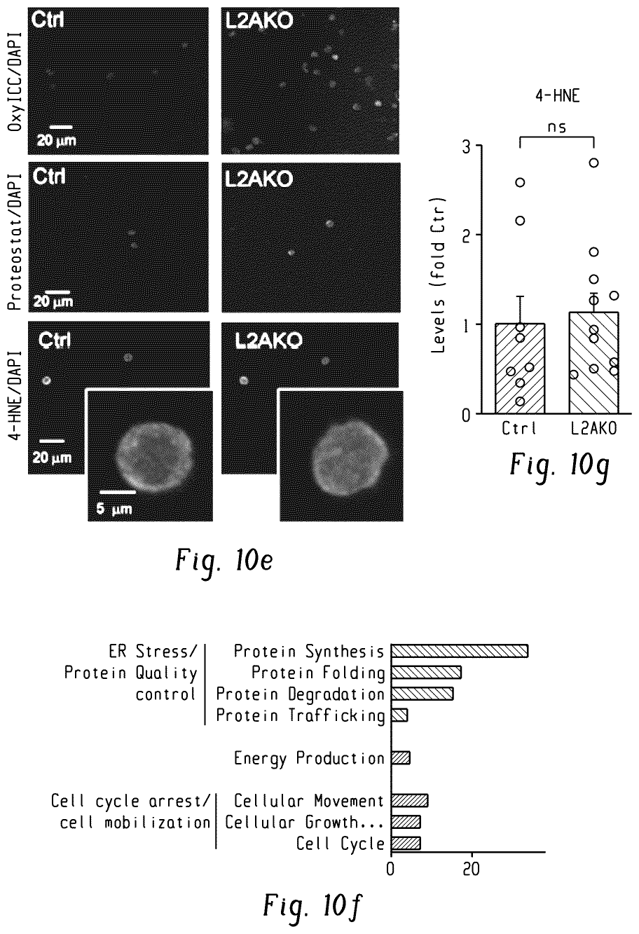

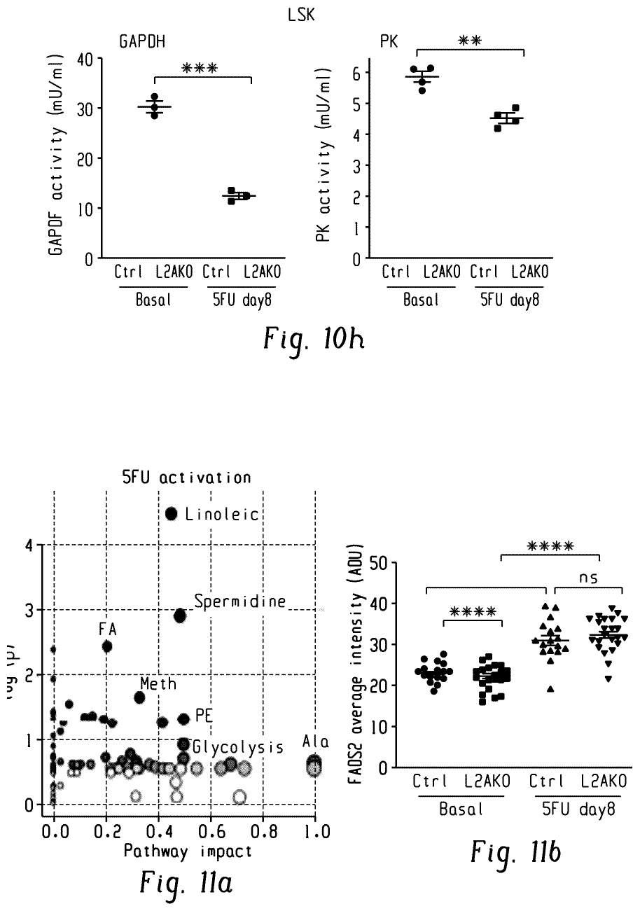

Comparative gene expression analysis of control and L2AKO HSC identified 405 genes differentially expressed during steady-state, and 855 genes following stem cell activation (FIG. 2e). Most transcriptional differences between resting control and L2AKO HSC were in genes related to cellular metabolism, cellular motility, cell cycle and proliferation; while upon HSC activation, differential expression occurred in genes with known roles in cell-to-cell signaling and interaction, cellular movement, and cell proliferation (FIG. 2f and FIG. 10a). Comparative quantitative proteomics also identified higher abundance of proteins related to metabolic pathways in L2AKO compared to control stem cells (FIG. 10b). To identify proteins degraded by CMA during stem cell activation, we focused on two groups of proteins: i) proteins whose levels decrease in CMA-competent control cells upon activation, and ii) proteins that accumulate in activated L2AKO cells (vs. activated control HSC). Notably, in the pool of proteins shared by both groups (300+8 proteins), the vast majority of them harbored CMA-targeting motifs (94.8+0.4% compared with the 84.7+8% in the total of 1,578+32 proteins (in average) detected in HSC by the proteomic analysis, p<0.05), further supporting that they are putative CMA substrates under these conditions (FIG. 2g,h). Proteins involved in metabolic pathways (mostly enzymes) were present in both groups of putative CMA substrates during stem cell activation (FIG. 6c). The fact that most changes in enzyme abundance during stem cell activation were only detected at the protein level (FIG. 2g) but not at the transcriptional level (FIG. 2f, right), suggests that CMA facilitates metabolic adaptation of activated HSC through direct degradation of metabolic enzymes.

### Example 6. Reduced Glycolysis in CMA-Deficient HSC

Metabolomics analyses of steady-state HSC revealed distinct metabolic phenotypes between L2AKO and control (partial least square-discriminant analysis and hierarchical clustering analysis in FIG. 2i, j). Pathway analysis of the top 25 metabolites significantly different between Ctrl and L2AKO HSC, highlighted a pronounced impairment in L2AKO cells of glycolysis and the glucose-alanine pathway, known energy sources critical for HSC quiescence (FIG. 2k and FIG. 10d). Extracellular metabolic flux analyses confirmed a decrease in glycolysis in L2AKO LSK cells compared to CMA-proficient controls, that was not observed for myeloid progenitor cells; thus, confirming that the defect in glycolysis upon CMA blockage is specific to stem cell-enriched cell populations (FIG. 2l,m and FIG. 10 e,f).

In agreement with the metabolomic and metabolic flux data, we found a significant decrease in the enzymatic activities of glyceraldehyde-3-phosphate dehydrogenase (GAPDH) and pyruvate kinase (PK)—two glycolytic enzymes previously identified as CMA substrates in other cell types in L2AKO stem cells compared to control cells (FIG. 2n). We attributed the reduced enzymatic activity to the higher content of oxidized residues per protein detected by mass spectrometry in these enzymes in steady-state L2AKO stem cells (FIG. 2o). We conclude that failure to degrade oxidized glycolytic enzymes in CMA-deficient stem cells leads to accumulation of non-functional enzymes and impaired glycolytic capability in these cells. Reduced quality control in L2AKO HSC is not limited to glycolytic enzymes, as we found a general loss of proteostasis in these cells. Indeed, mass spectrometry analysis revealed that CMA-deficient stem cell enriched LSK have significantly higher levels of carbonylated peptides and oxidized proteins (including reversible and irreversible oxidation of sulfur-containing amino acids, methionine and cysteine—collectively plotted in FIG. 2p). Direct cellular staining also confirmed the higher content of oxidized proteins and protein inclusions in L2AKO HSC (FIG. 2q,r and FIG. 10g). In agreement with the fact that CMA exclusively degrades proteins, we found that oxidized products accumulating in L2AKO HSC seem to be preferentially proteins and peptides, while for example, lipid peroxidation products appeared unaltered (FIG. 10g,h). Analysis of the subset of proteins bearing CMA-targeting motifs that display higher oxidation in L2AKO HSC identified besides glycolytic enzymes, other proteins related to cellular energetics, proteins involved in cytosolic and ER protein quality control (synthesis, folding, degradation and trafficking) — which could explain the dilated ER observed in L2AKO HSC cells (FIG. 9c,g)—and proteins that contribute to cell cycle and cellular mobilization (FIG. 10i,j).

Together, our results support that loss of CMA leads to defective protein quality control in quiescent HSC and reduces the ability to accommodate the metabolic requirements for appropriate stem cell activation.

### Example 7. CMA Regulates Lipid Metabolism in Activated HSC

Metabolomic analysis of 5FU-activated LSK cells revealed that glycolysis was also impaired in CMA-incompetent cells during activation, likely through similar mechanisms, since we also found higher rates of oxidation and of metabolic markers of oxidant stress (methionine sulfoxide, xanthine and spermine), higher levels of damage-related PTMs and reduced activity of glycolytic enzymes in L2A KO stem cells than in control upon activation (FIG. 2p and FIG. 10k, l).

However, the most noticeable metabolic differences between CMA-competent and CMA-incompetent stem cells during activation occurred in pathways related to lipid metabolism, with linoleic acid metabolism and fatty acid biosynthesis and beta-oxidation among the top affected pathways (FIG. 3a-d and FIG. 11a). Activation of control LSK cells associated with higher flux through linoleic and a-linolenic acid metabolism, whereas in activated L2AKO LSK we observed accumulation of the two precursors (α-linolenic and linoleic acid) and very pronounced decrease in all downstream metabolites (FIG. 3b-d). Blocking linoleic acid metabolism with an inhibitor of fatty acid desaturase 2 (FADS2), the rate limiting enzyme of this pathway, was sufficient to reduce self-renewal ability of control cells (FIG. 3e); while conversely, treatment of CMA-defective HSC with γ-linolenic acid (GLA), the direct product of FADS2, restored their self-renewal capacity ex vivo (FIG. 3f). Thus, the blockage in a-linolenic and linoleic acid metabolism is of functional relevance in L2AKO HSC.

To elucidate how CMA may regulate linoleic acid metabolism, we focused on FADS2 because the blockage in this pathway in CMA-deficient cells occurred at the step catalyzed by this enzyme (FIG. 3c shows the steps, metabolites and regulatory enzymes in linoleic and a-linolenic acid metabolism and FIG. 3b,d show accumulation of the precursors of the reaction catalyzed by FADS2 and reduction of the downstream products). FADS2 content increased upon 5FU activation at comparable levels in control and L2AKO LSK cells (FIG. 11b,c), suggesting that qualitative rather than quantitative differences may be behind the lower activity of FADS2 in CMA-deficient cells. Among the different posttranslational modifications that could modulate FADS2 activity, we focused on acetylation because the following findings support an important role for acetylation in the CMA-dependent proteome remodeling of HSC and progenitor cells upon activation: i) FADS2 would acquire a CMA-targeting motif if lysine 42 becomes acetylated (38VIDRK42 changes to VIDRQ-like) (FIG. 3g); ii) CMA-targeting motifs that can be generated by acetylation were significantly enriched in the 300 proteins that decreased in control cells upon activation and remain elevated in L2AKO HSC (FIG. 11d), iii) mass spectrometry demonstrated a trend toward higher acetylation in putative CMA substrates (KFERQ-like motif containing proteins) among the proteins that accumulate in L2AKO stem cells upon activation (FIG. 11e); and iv) the pools of proteins with higher acetylation in L2AKO LSK cells and those with predicted acetylation generated CMA-targeting motifs in proteins that accumulate in L2AKO HSC and in human HSC from old individuals, were all enriched in proteins involved in cellular metabolism, including lipid metabolism (FIG. 11d, e).

Mass spectrometry analysis identified higher acetylation of the FADS2 K42 residue in L2AKO LSK cells upon 5FU activation (FIG. 3g). Single point mutagenesis in this residue confirmed that an acetylation mimetic form of FADS2 (FADS2VIDRQ) when expressed in HSC was rapidly degraded in lysosomes (FIG. 3h) in a L2A-dependent manner (FIG. 3i), and that this rapid degradation of FADS2 by CMA was no longer observed in an acetylation-resistant mutant at that residue (FADS2VIDRA) (FIG. 3h,i). These results support that, as previously described for other enzymes, acetylation triggers removal of inactive forms of FADS2 by CMA and that, by changing the active/inactive enzyme ratio, CMA positively regulates FADS2 activity and increases linoleic and ct-linolenic fatty acid metabolism during HSC activation.

As expected from HSC unable to upregulate linoleic acid metabolism during activation, L2AKO cells showed lower fatty acid (3-oxidation related mitochondrial respiration rates than control cells (FIG. 3j, k). Reduced lipid β-oxidation can explain the lower ATP levels in L2AKO stem cells upon activation (FIG. 2b) and their reduced self-renewal ability, as supplementation with methyl-pyruvate (to bypass reduced β-oxidation) significantly increased L2AKO HSC function ex vivo (FIG. 3l). Furthermore, a toxic effect of unmetabolized linoleic and ct-linolenic fatty acids — known to increase oxidative and endoplasmic reticulum (ER) stress—also contributes to the reduced function of L2AKO HSC, as they display higher ROS levels (FIG. 2c,d) and abnormally dilated ER (FIG. 9c,g), and addition of the ROS scavenger N-acetylcysteine (NAC) restored their ex vivo long-term colony initiation abilities (FIG. 3m). These data support that the compromised function of CMA-deficient HSC is likely a combination of the cells' inability to meet the energetic requirements of activation and the toxic effects of accumulating unmetabolized, pro-oxidative lipid products.

Comparative proteomic analysis revealed that L2AKO HSC from young mice phenocopy in part the changes in the proteome of old control HSC. In fact, almost half of the proteins that we found that accumulated in HSC from 25 m mice were also elevated under basal conditions in HSC from 4 m L2AKO mice and they fell in similar functional family categories (FIG. 11f). Similarly, analysis of the metabolic phenotypes and hierarchical clustering analysis of the top 50 metabolites significantly different between HSC from young and old mice demonstrated consistent changes in glycolysis and in metabolism of free fatty acids with those observed in HSC from young L2AKO mice (FIG. 11g-i). Furthermore, the ratio of GLA to LA (metabolite and precursor in the step catalyzed by FADS2) was significantly reduced in LSK cells from old mice (FIG. 3n) and the acetylation state of FADS2 at the K42 residue in these cells was significantly elevated (FIG. 3o) when compared with LSK from young mice.

Overall, these findings support a possible similar molecular mechanism underlying the metabolic derangement of L2AKO HSC and old HSC. To test the possible contribution of the observed failure in linolenic and ct-linolenic fatty acid metabolism to the functional deficiencies of old HSC, we subjected 22m mice to daily injections of GLA for 7 weeks. We observed that in vivo GLA supplementation improved HSC function as assessed by colony formation (FIG. 3p). Furthermore, ex vivo supplementation with GLA also increased the number of functional human stem cells in long-term cultures of CD34+ cells derived from old (>65 years) patients (FIG. 3q). These findings further support the relevance of linoleic and ct-linolenic fatty acid metabolism for HSC activation both in mice and in humans, the contribution of defective flux through this pathway to HSC loss of function during aging and the similarity, at the molecular level, between the changes in FADS2 proteostasis in HSC in aging and in our model of deficient CMA.

### Example 8. CMA Activation Preserves HSC Function in Aging

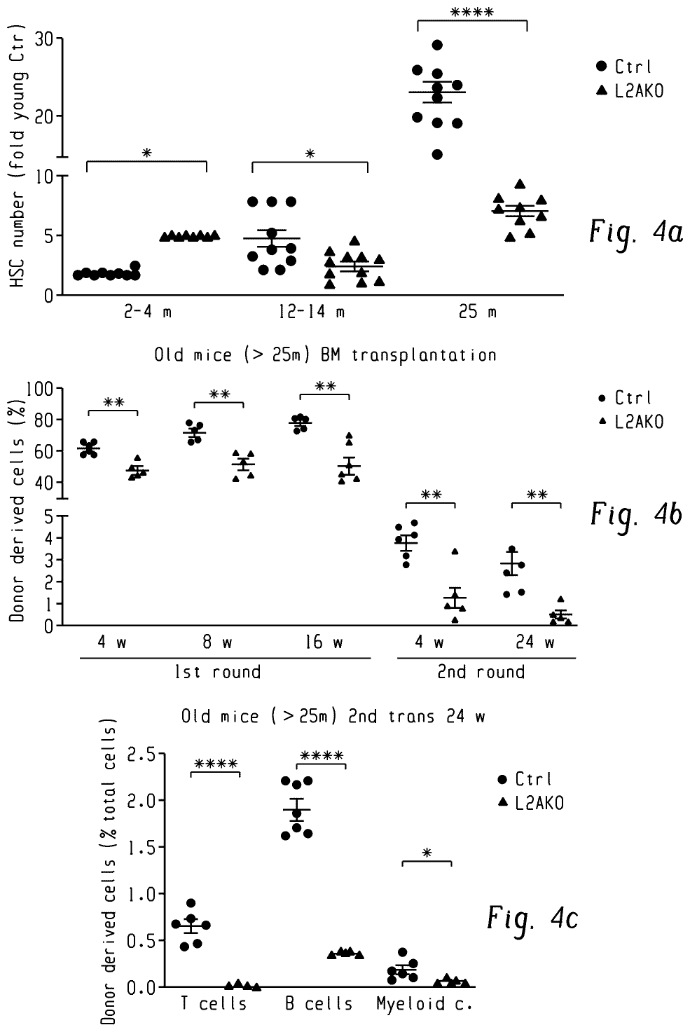

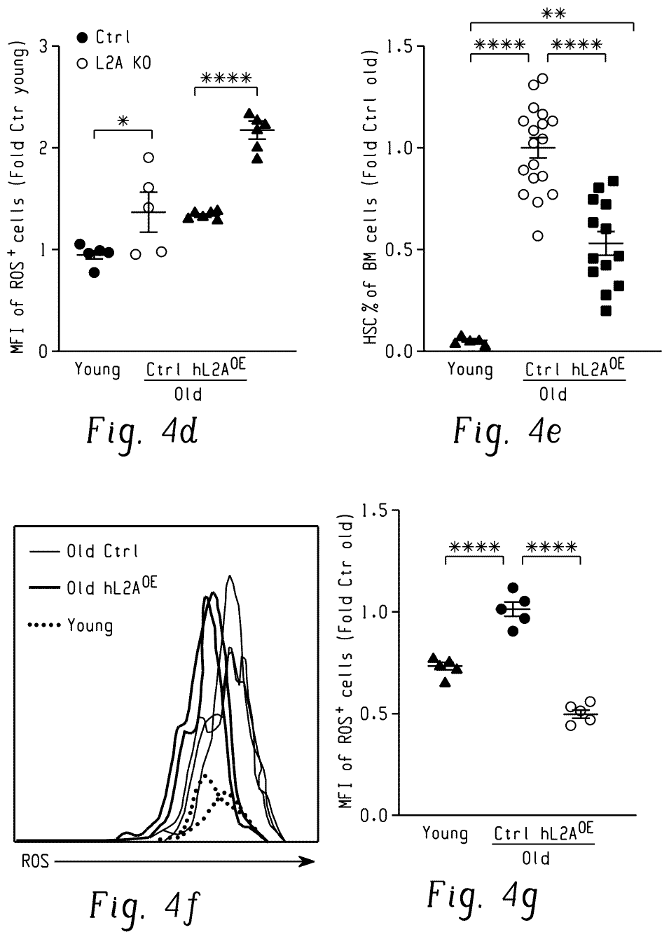

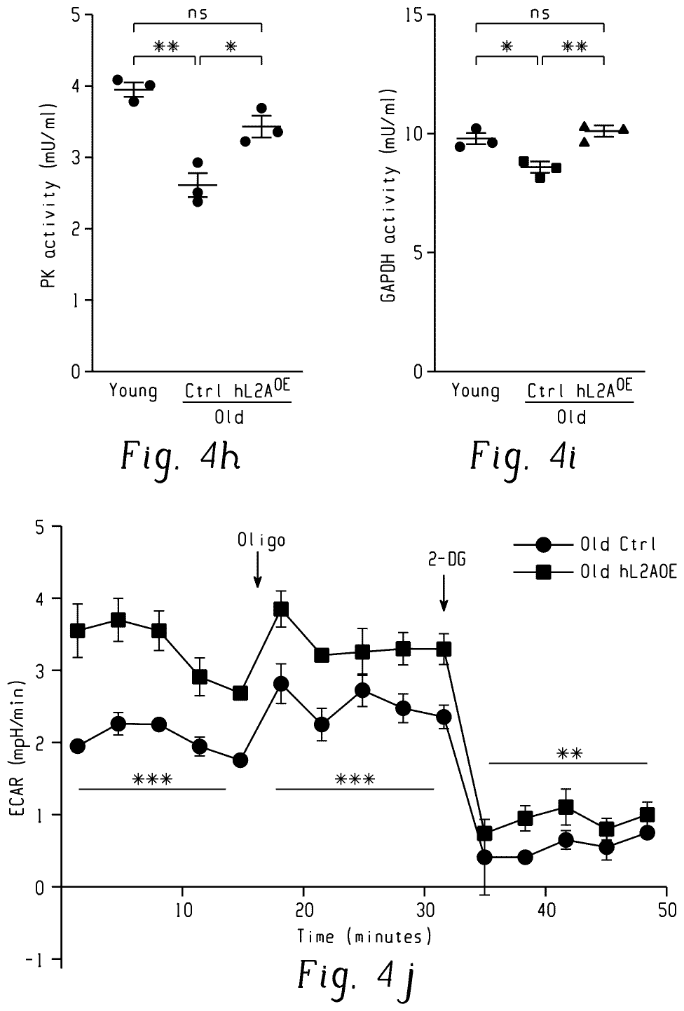

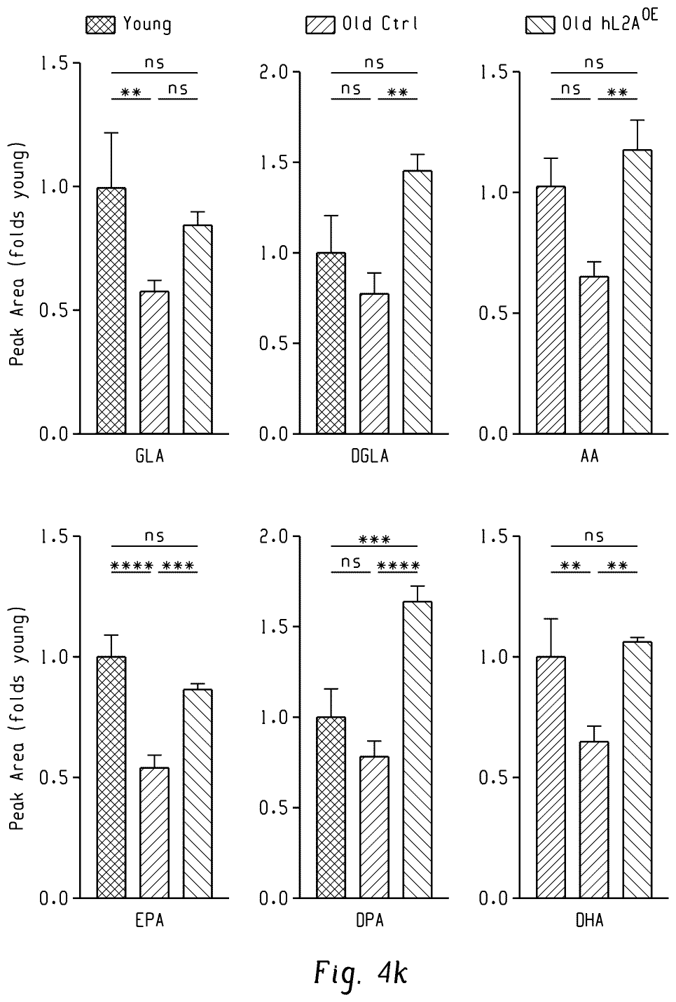

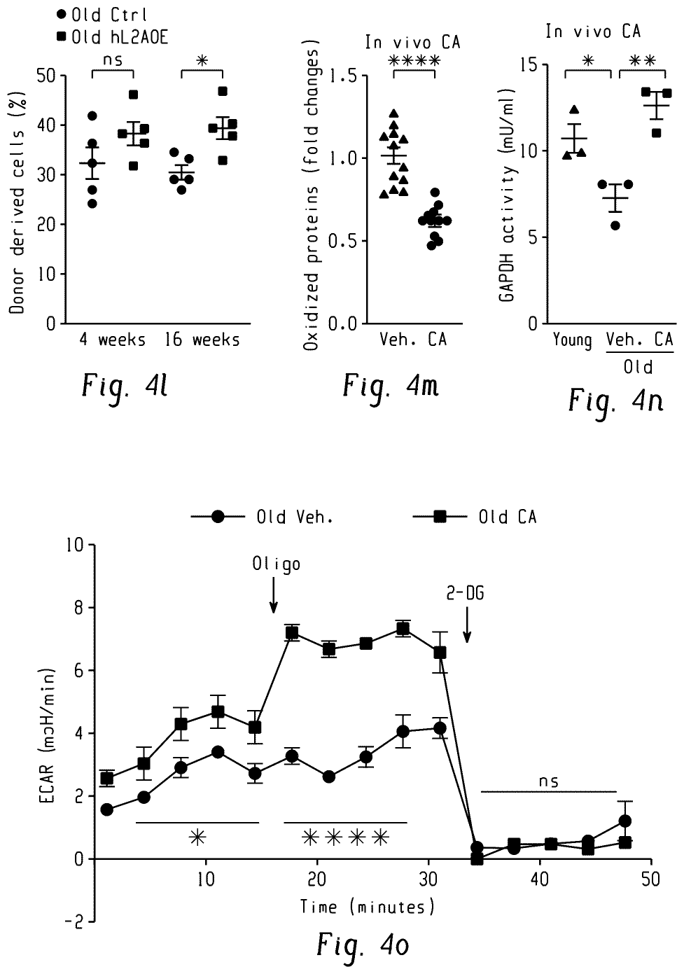

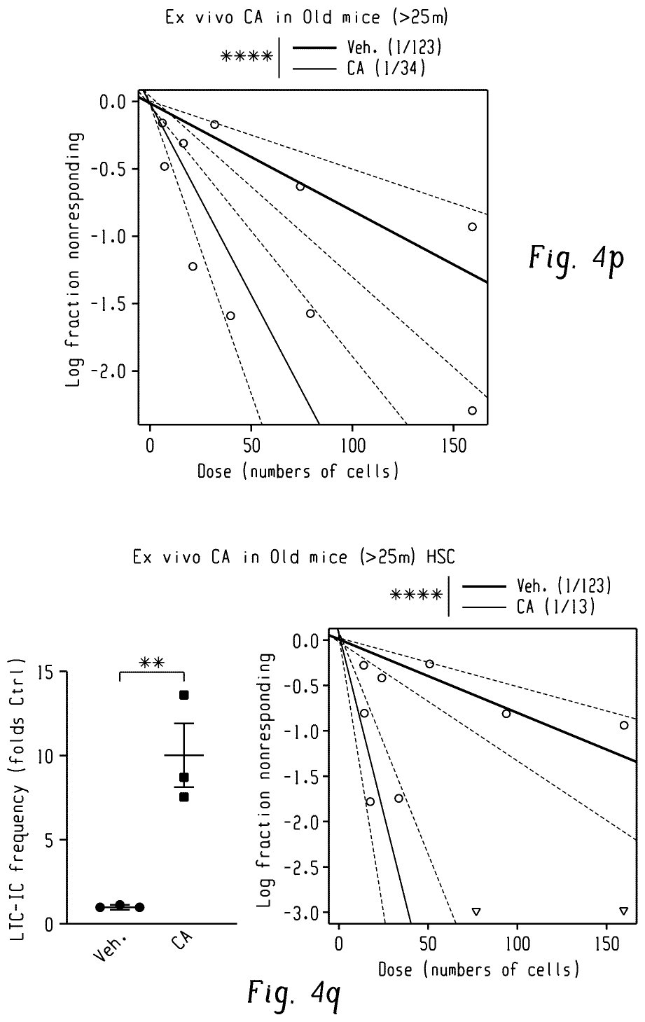

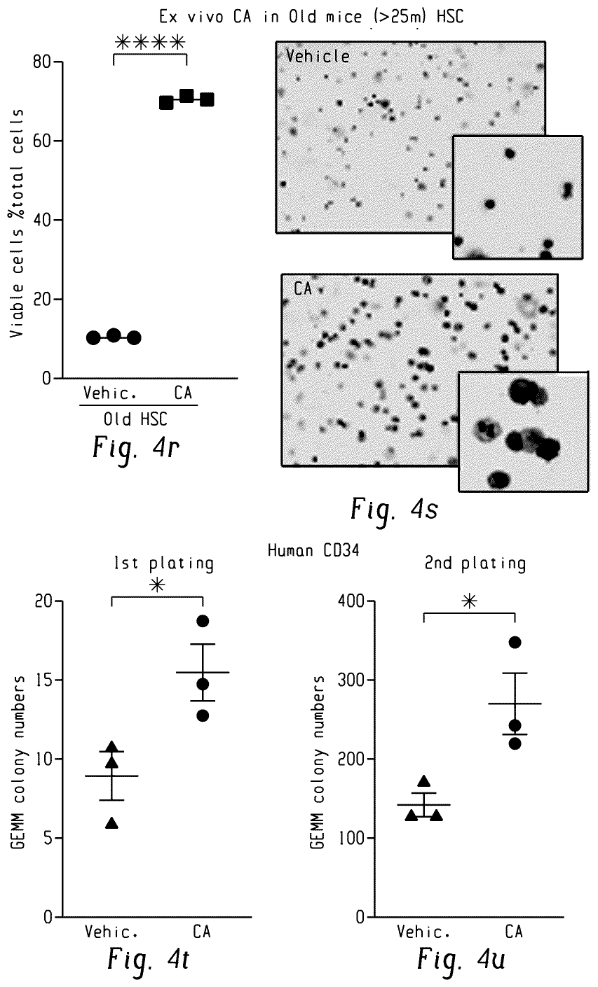

We observed that the progressive depletion of functional HSC in absence of CMA accentuates with age as evidenced by: 1) reduced numbers of phenotypical HSC (FIG. 4a), 2) decreased HSC reconstitution ability noticeable in HSC from old L2AKO mice already upon the first round of transplantation (FIG. 4b,c), in contrast to young L2AKO mice that only displayed differences in reconstitution abilities after the second round of transplantation (FIG. li); and 3) more pronounced age-dependent increase in intracellular ROS levels in L2AKO mice HSC compared to wild type littermate mice (FIG. 4d).

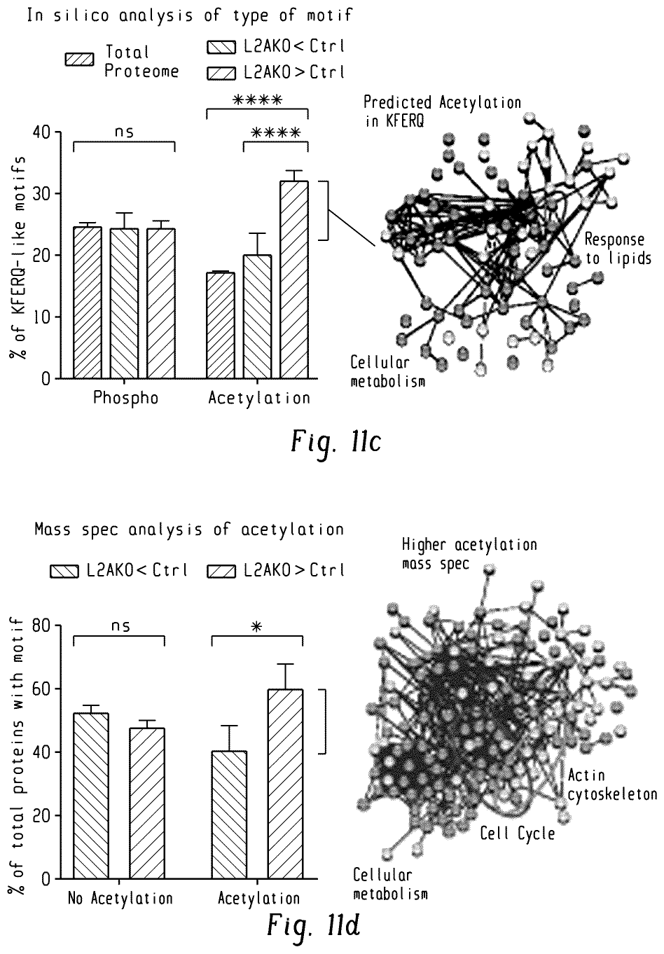

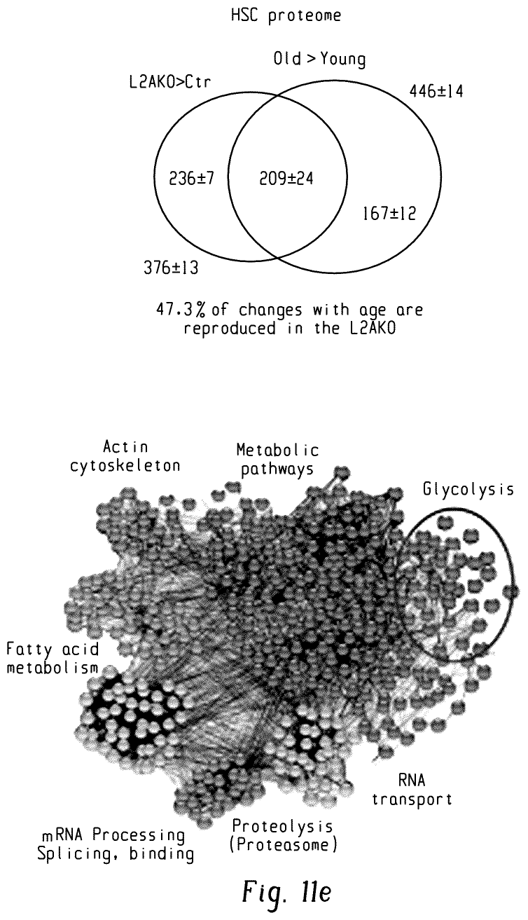

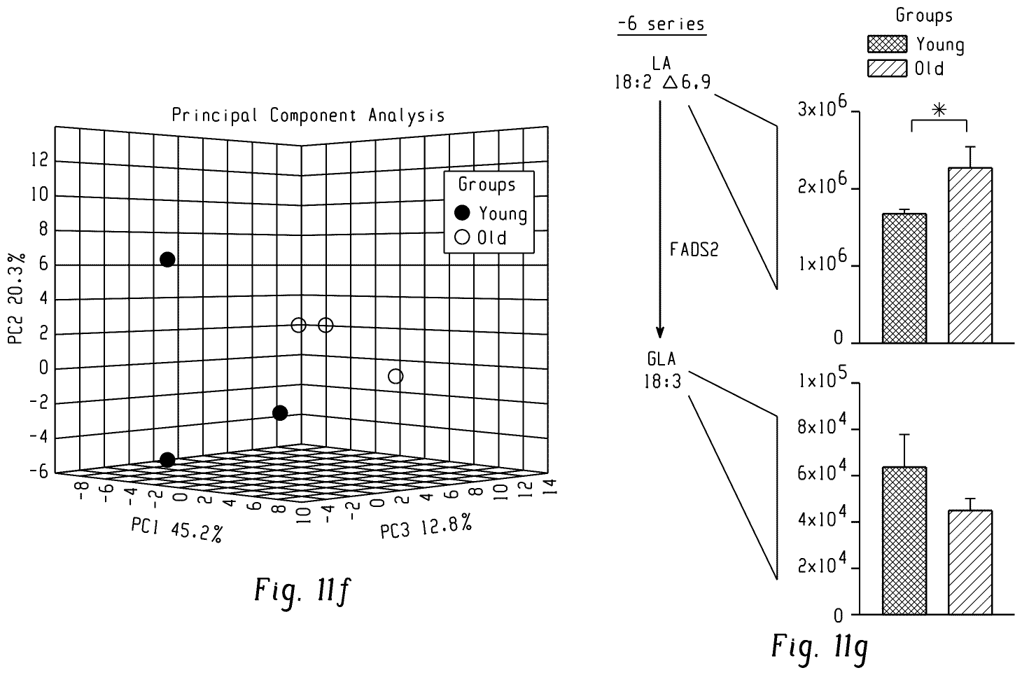

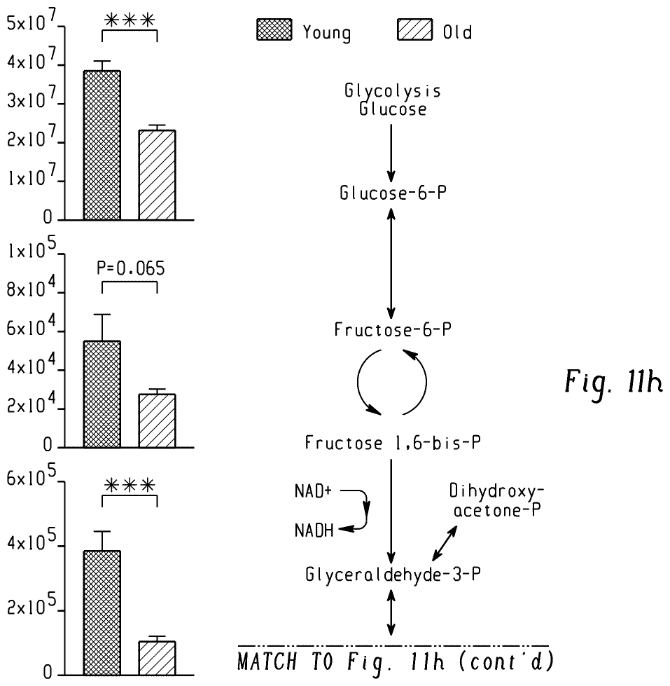

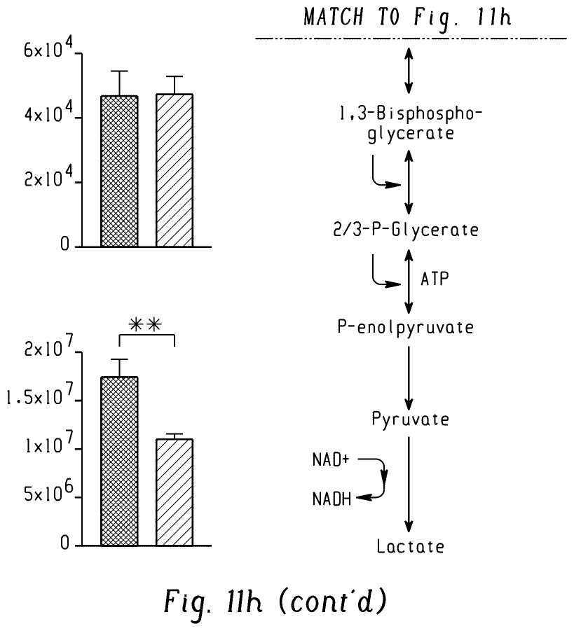

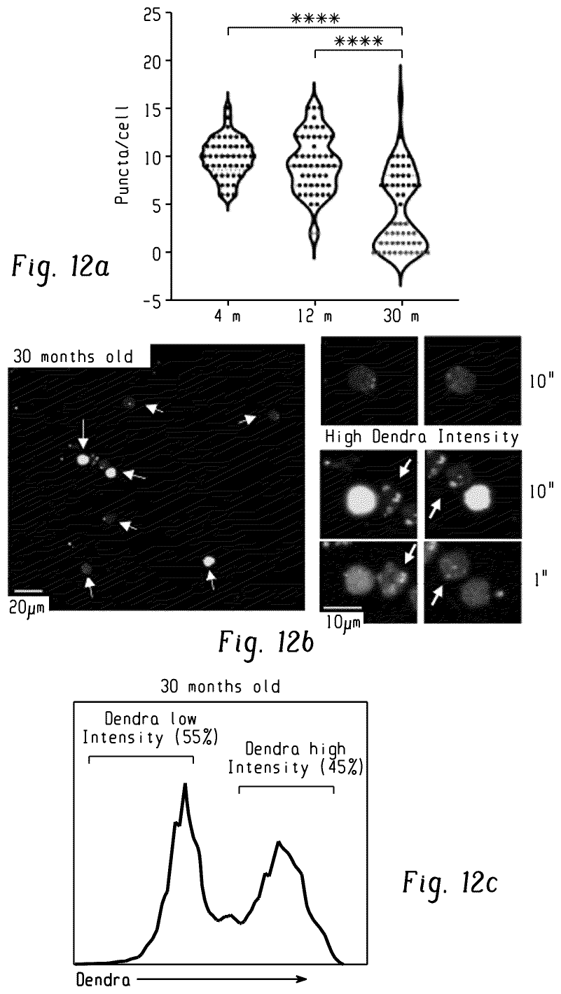

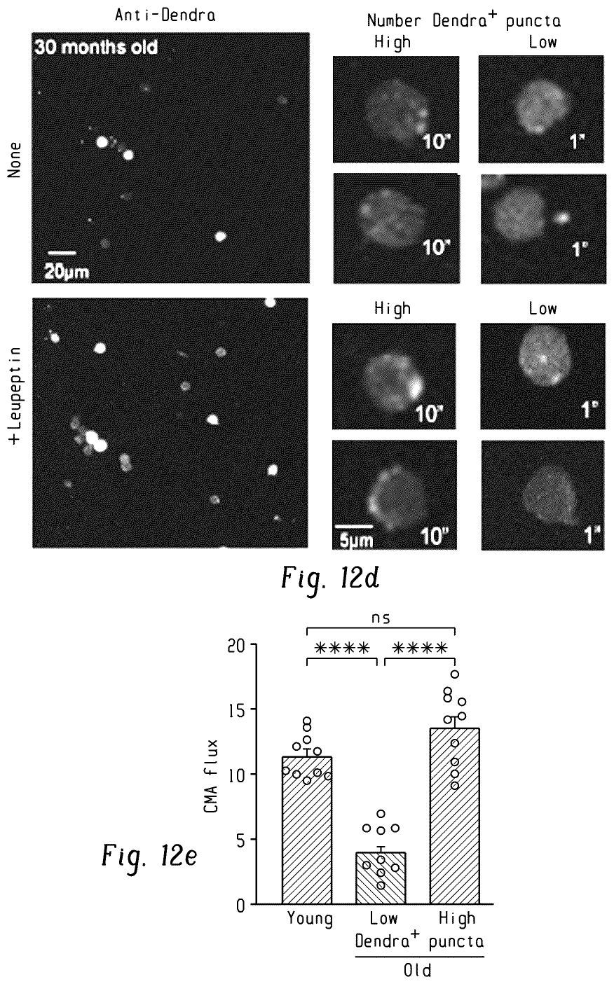

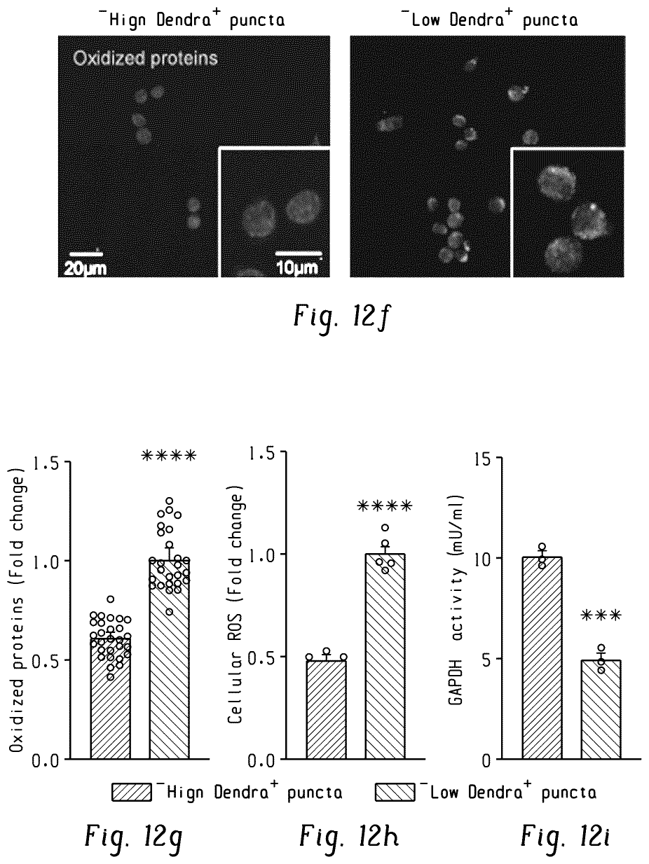

To gain a better understanding of the observed changes in CMA activity in HSC with age (FIG. 1a,b) and to place them in the context of the well-known functional heterogeneity of the aged HSC24, we next explored in more detail CMA activity in old HSC. Re-analysis of CMA activity in HSC from young and old KFERQ-Dendra mice, by plotting the absolute number of Dendra+ puncta per cell (instead of averaging number of Dendra+ puncta for all HSC), revealed the emergence of two groups of HSCs with very different CMA activity (FIG. 8a). Immunostaining images and FACS both demonstrated high levels of cytosolic KFERQ-Dendra protein in about 45% of cells, which corresponded to those with low number of fluorescent puncta (FIG. 12b,c). Reduced number of Dendra+ puncta and increased cytosolic staining in some old cells was a direct consequence of reduced flux through CMA, that we measured upon blocking protein degradation in the lysosomal lumen with leupeptin (FIG. 12d,e); notably, HSC cells from old mice with low cytosolic Dendra accumulation displayed CMA flux almost indistinguishable from young HSC (FIG. 12d,e). In agreement with the observed disruption of proteostasis upon CMA failure, we found that the population of HSC from old mice with low CMA activity had significantly higher levels of oxidized proteins, higher cellular ROS levels and displayed lower GAPDH activity than HSC with preserved CMA from the same mice (FIG. 12f-i). These data further support the contribution of reduced CMA with age to loss of HSC proteostasis.

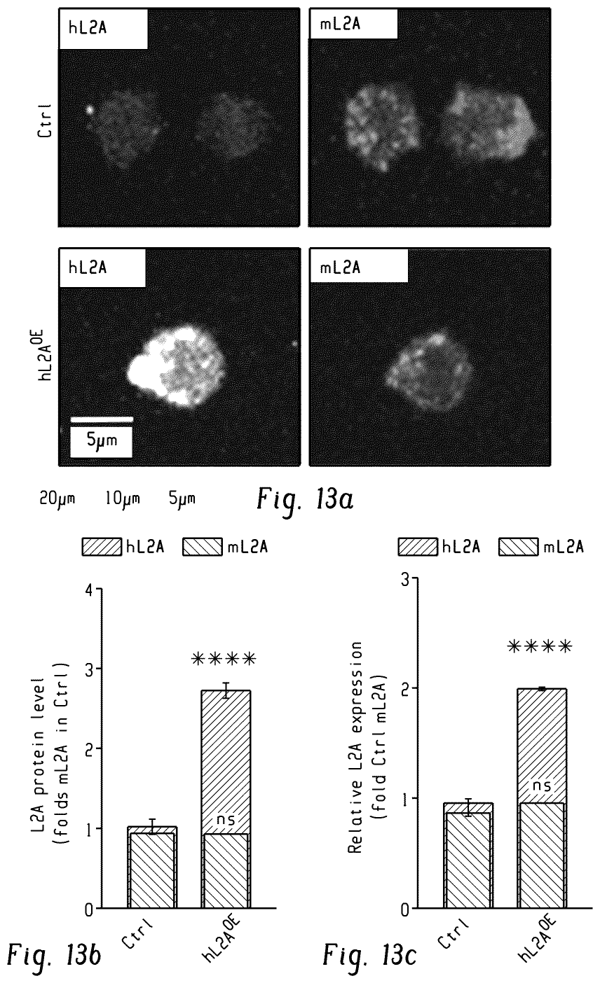

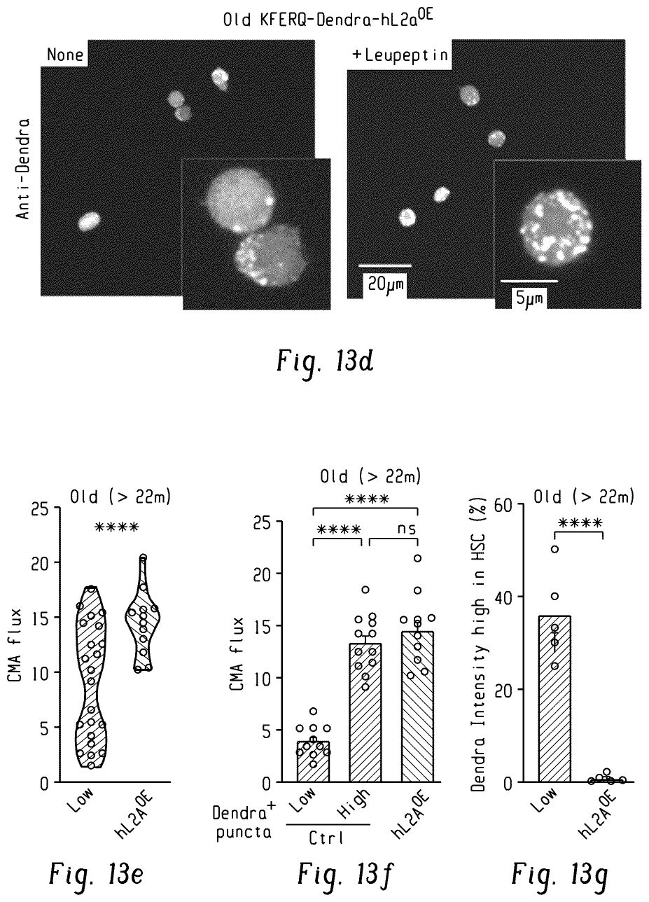

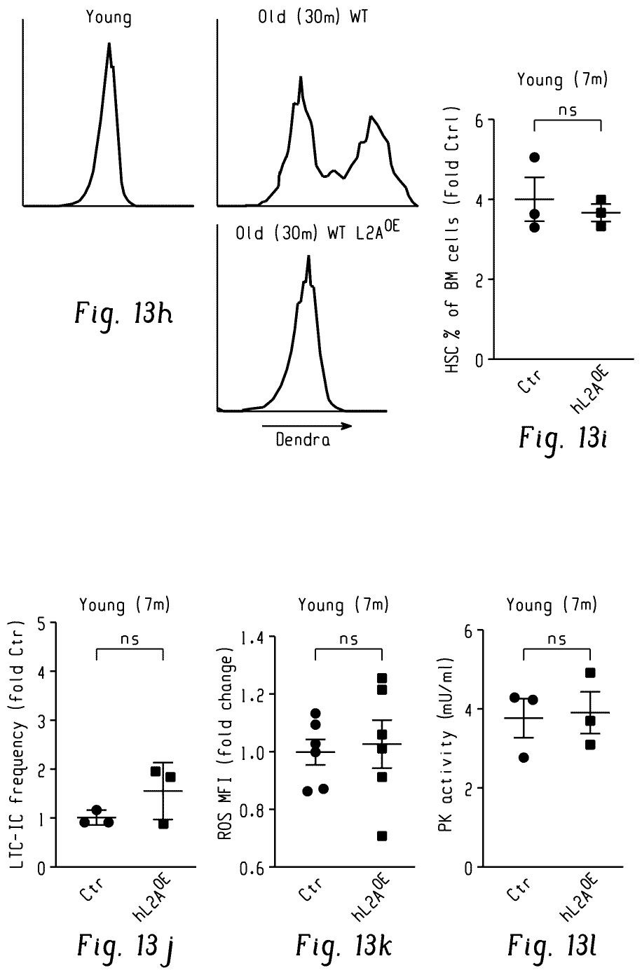

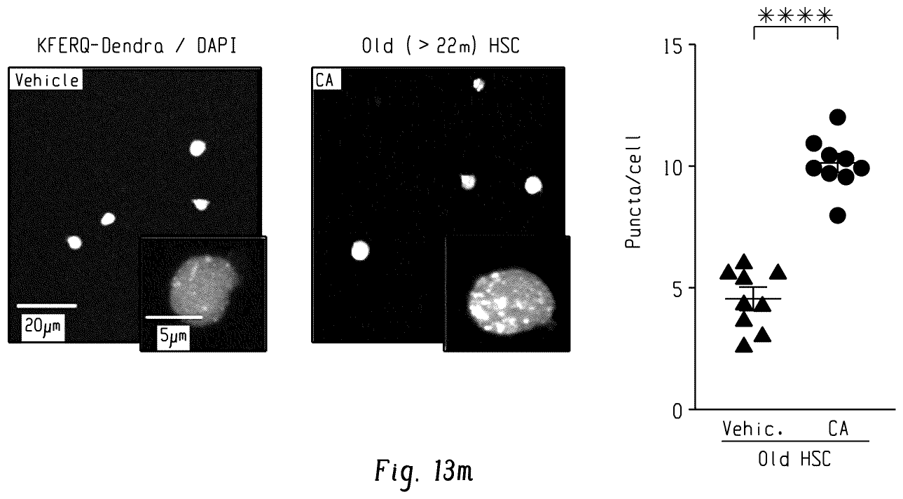

To test whether preventing the age-dependent decline in CMA could preserve HSC function, we used ERCre-hL2A mice (hereafter hL2AOE) (in which expression of human L2A was induced at middle life (12 months of age) for the rest of their life to compensate for the decrease in endogenous L2A levels responsible for the decline in CMA activity with age. We confirmed expression of hL2A, at the mRNA (one hL2A transcript for each mL2A) and protein level, without affecting levels of the endogenous mouse L2A (FIG. 13a-c). We tested whether expression of hL2A was effective in preserving normal CMA activity until late in life by crossing KFERQ-Dendra mice with the hL2AOE mice. HSC from old (>22 m) KFERQ-Dendra-hL2AOE mice displayed significantly higher CMA flux than control littermate and CMA activity was more homogeneous among old stem cells, to the point that the population with low CMA activity observed in control old mice was undetectable in hL2AOE mice (FIG. 13d-h).

Contrary to young animals where the pool of stem cells is small, but the potency of each stem cell is high, aging associates with a well-characterized expansion of the stem cell population but of restricted functionality at the single cell level. Analysis of the BM of old hL2AOE mice revealed that the frequency of HSC in BM from old hL2AOE mice was significantly lower than in control old littermates (FIG. 4e). We predicted that the age-dependent expansion of the HSC pool was no longer occurring in these mice due to their preserved better functionality at the single cell level. To confirm this hypothesis, we directly analyzed quality and function of HSC and found that HSC cells from old hL2AOE mice displayed significantly lower intracellular ROS levels (FIG. 4f,g), PK and GAPDH activities closer to those observed in young mice (FIG. 4h,i) and higher glycolysis rates than control old littermates (FIG. 4j). Metabolomic analysis showed that maintenance of functional CMA in old mice also prevented the significant decrease in FADS2-generated polyunsaturated fatty acids observed in the old control group (FIG. 4k). The better proteostasis and preserved glucose and fatty acid metabolism observed in HSC from old hL2AOE mice is probably responsible for their improved functionality, detected as their higher efficiency in BM reconstitution upon transplantation (FIG. 4l).

Overall, contrary to CMA blockage which reduces HSC frequency and functionality, preservation of CMA activity until late in life, prevented the loss of HSC function with age and the expansion of poorly functional HSC observed in control old mice. Upregulation of L2A expression in young (4 m) hL2AOE mice for 3 m did not change HSC frequency in BM, HSC performance in the LTC-IC assay or ROS levels and PK activity of HSC (FIG. 13i-l), supporting a maximal beneficial effect of CMA upregulation in conditions with an already underlying CMA defect, such as aging.

### Example 9. Restoration of CMA Rejuvenates Aged HSC

Lastly, to explore if upregulation of CMA in already old HSC could still be a viable intervention to improve their function, we exposed HSC from old mice to a well-characterized pharmacological CMA activator (CA) optimized for in vivo use 61. We confirmed that CA was still effective in activating CMA in HSC from old KFERQ-Dendra mice (FIG. 9m), and demonstrated that daily oral administration of CA (20 mg/kg b.w.) for only 2 months was sufficient to reduce levels of oxidized proteins, restore GAPDH activity and increase glycolytic flux (FIG. 4m-o and FIG. 13n), which likely contributes to the remarkable increase in HSC function observed upon CA administration (shown as better performance in the LTC-IC assay) (FIG. 4p). To confirm that the functional improvement of HSC was due to direct activation of CMA in these cells and independent of any other systemic beneficial effect of CA when administered systemically in vivo, we exposed HSC from old mice to CA in vitro and found a similarly striking increase in the number of LTC-IC and higher ability of stem cells to generating viable mature cells of various lineages compared to vehicle treated controls (FIG. 4q-s).

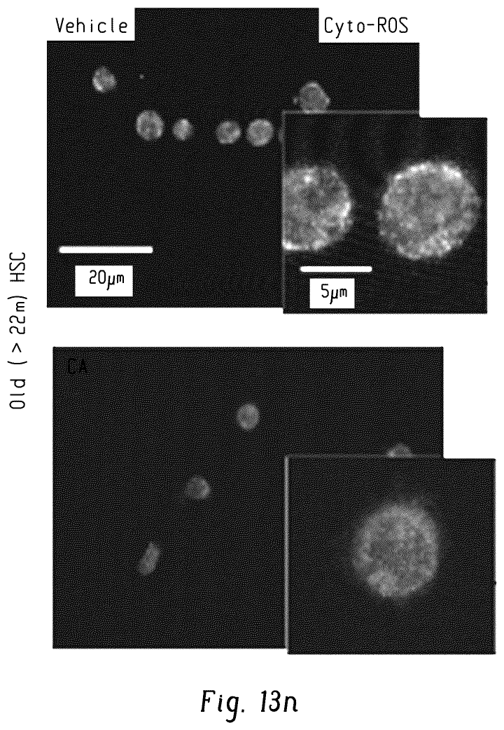

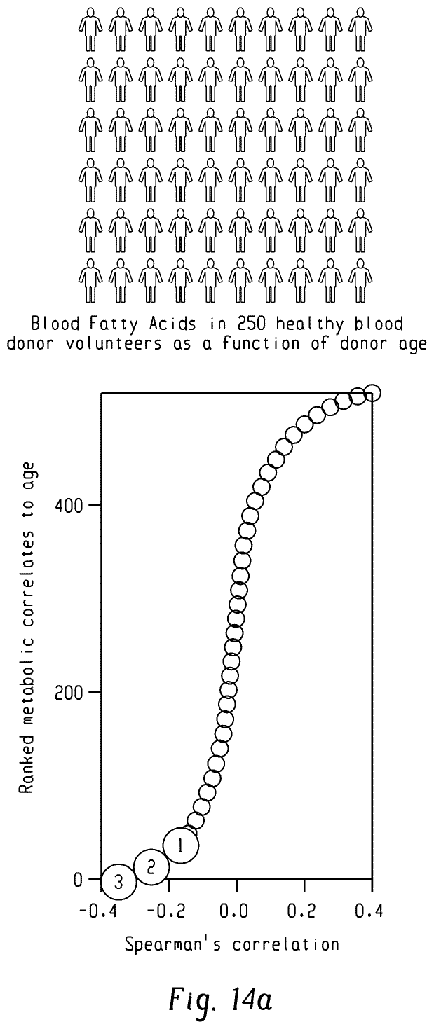

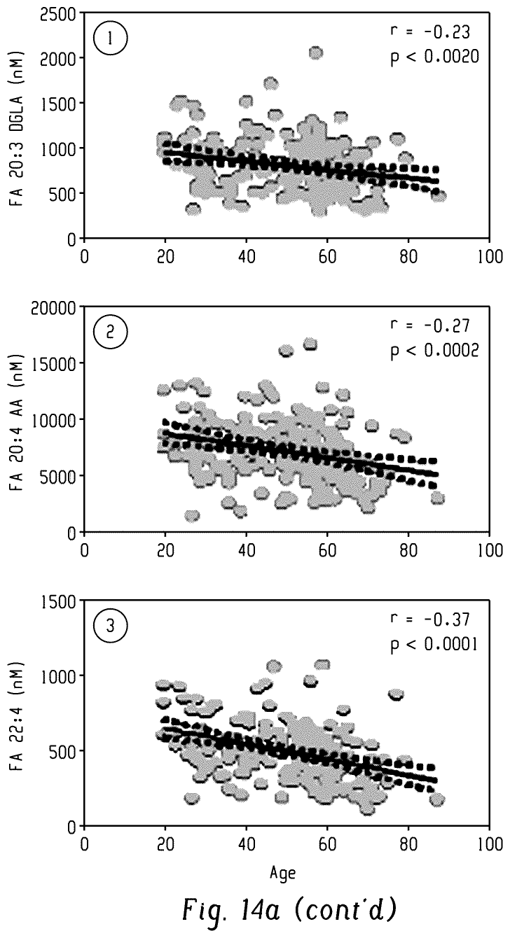

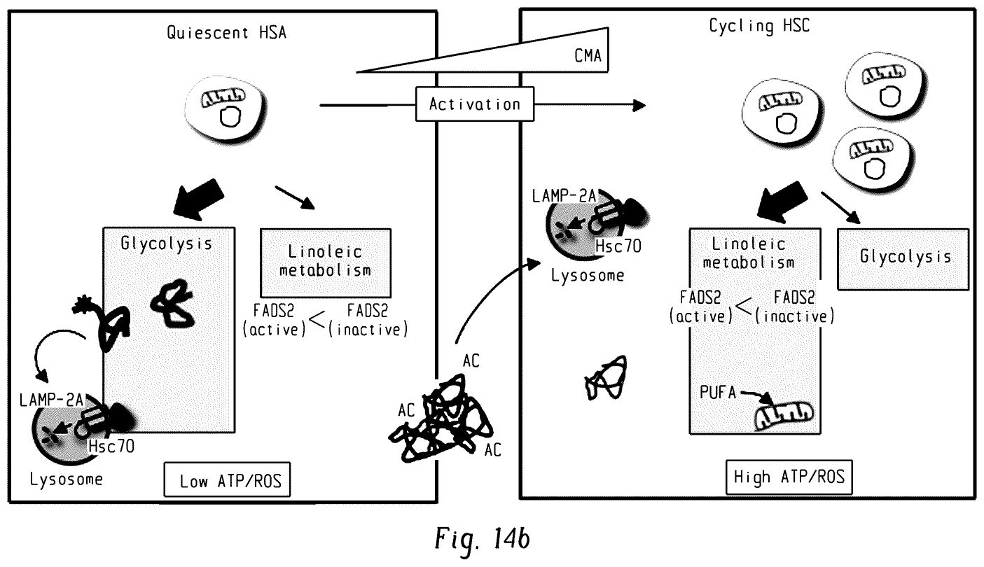

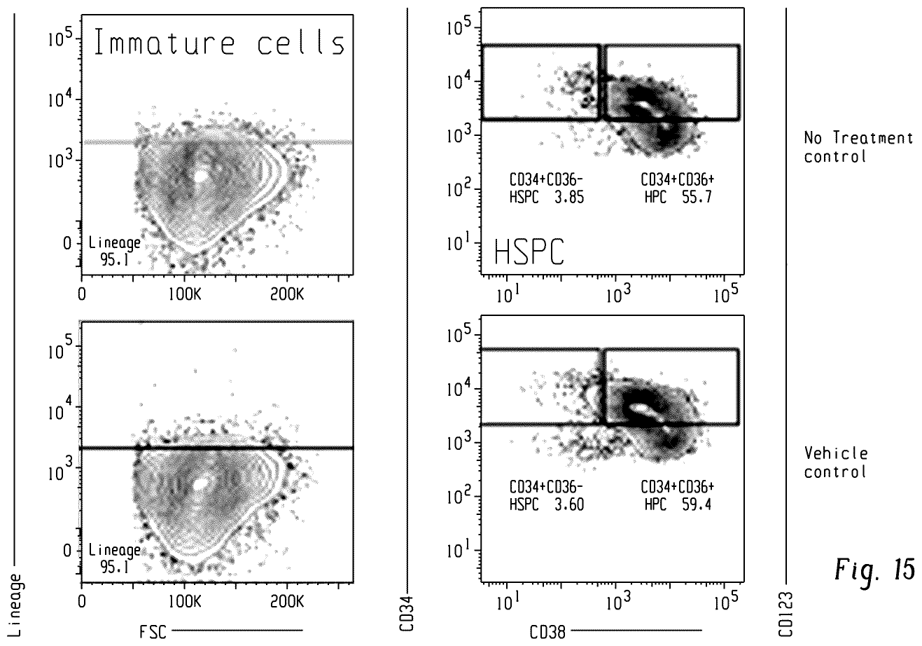

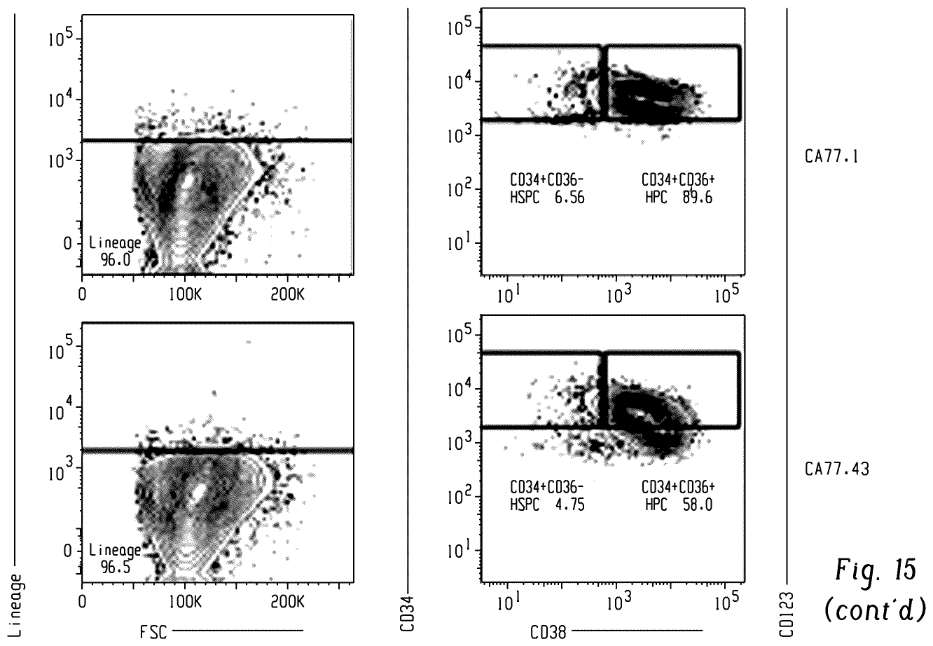

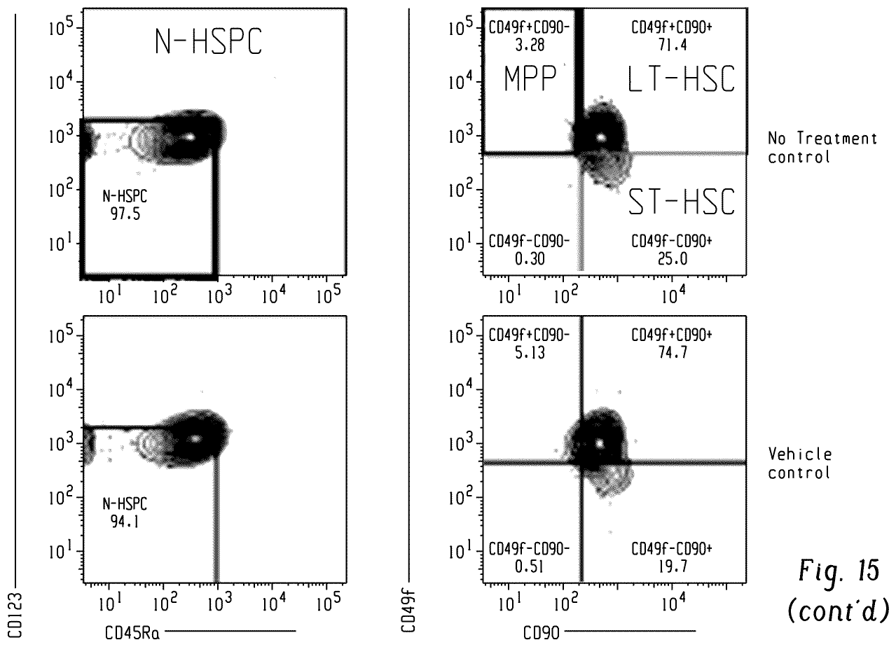

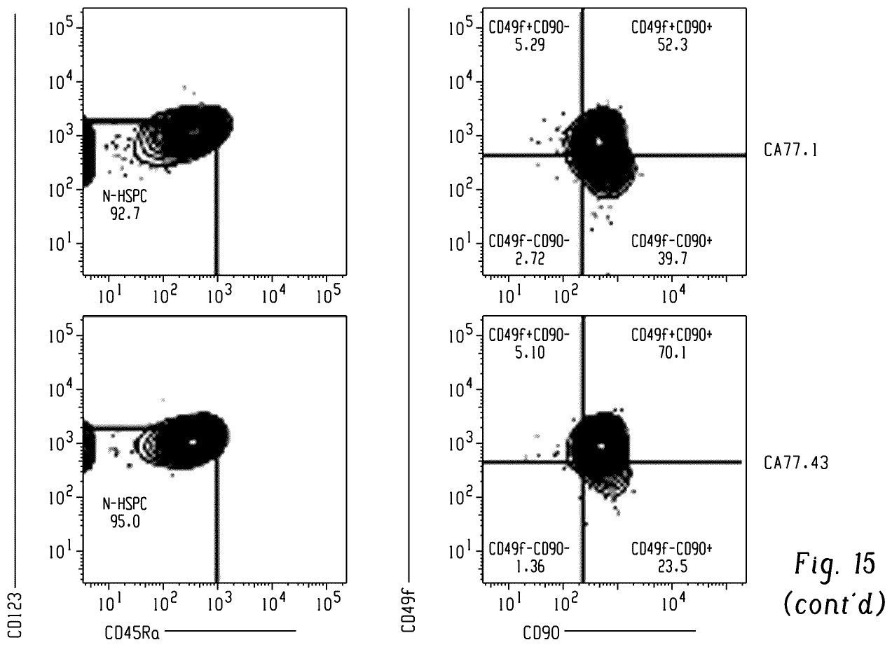

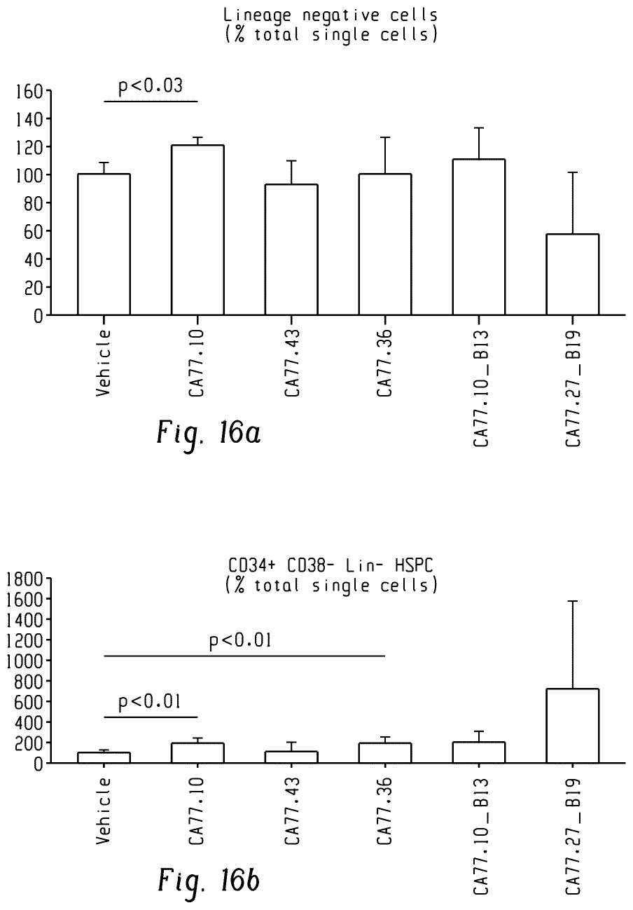

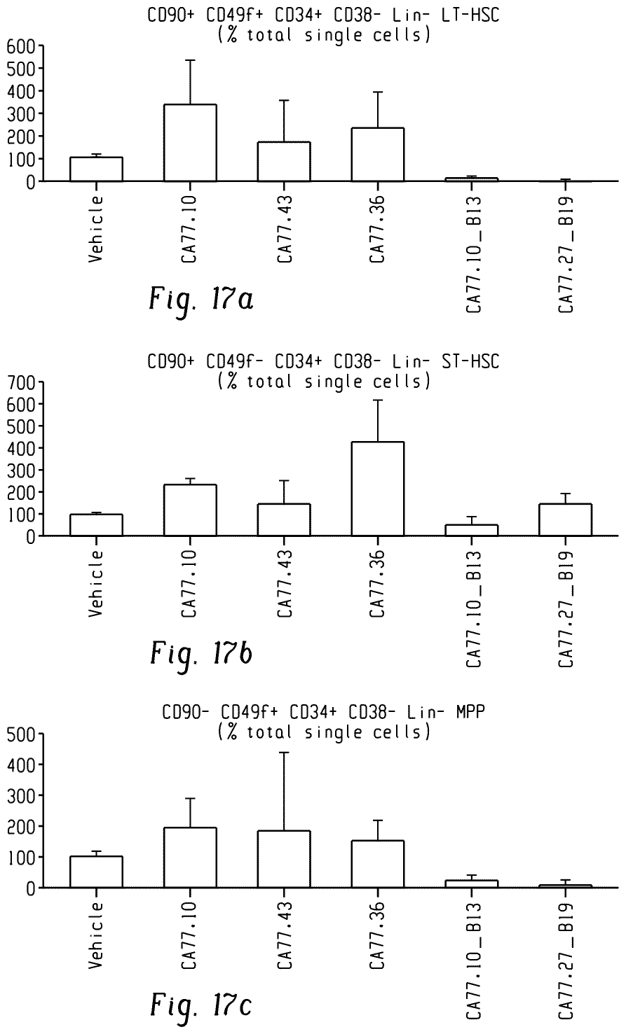

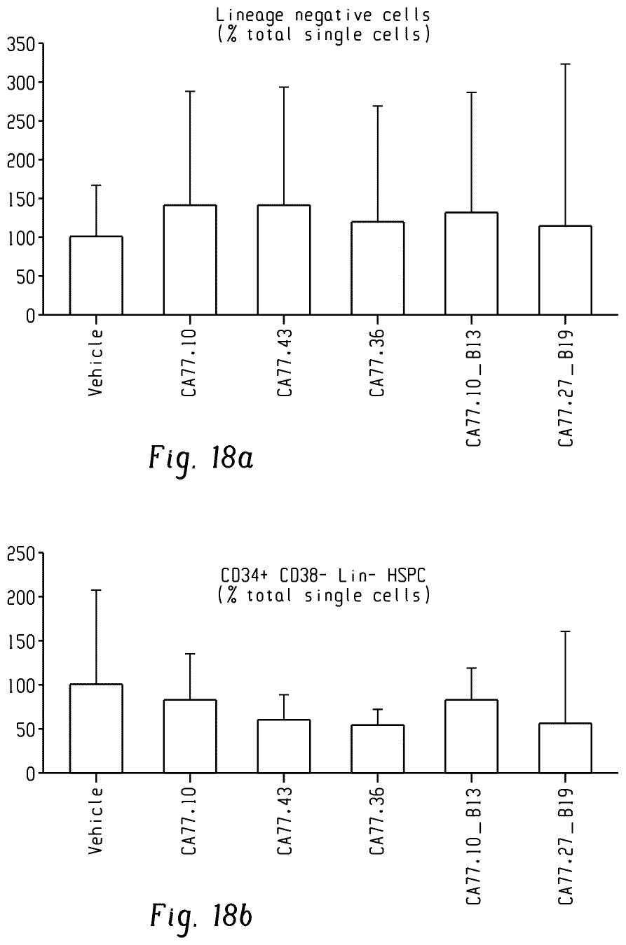

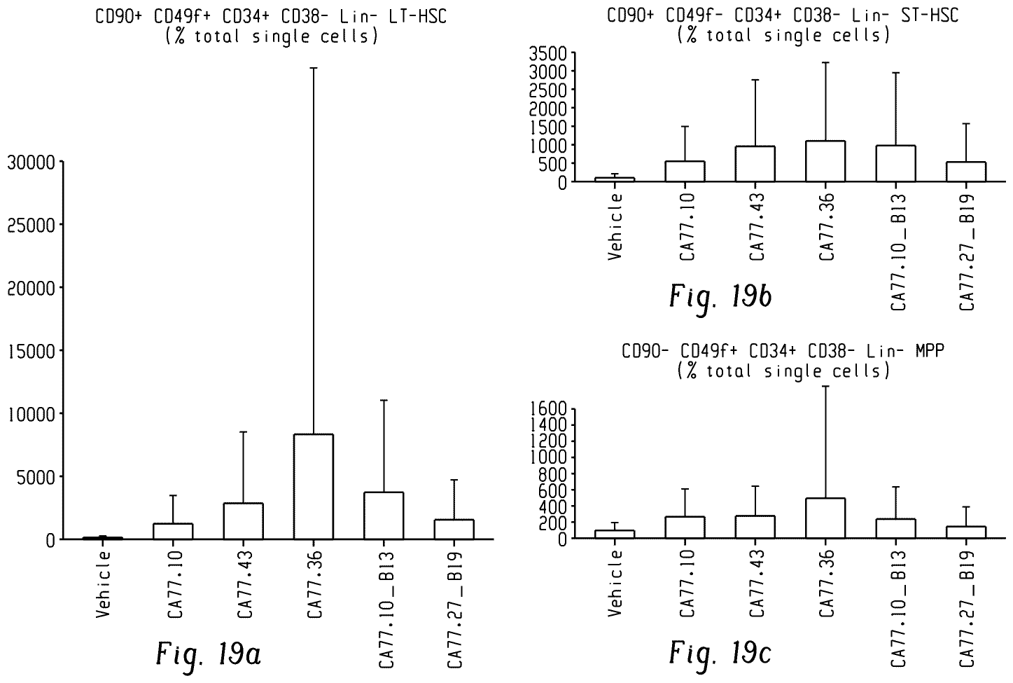

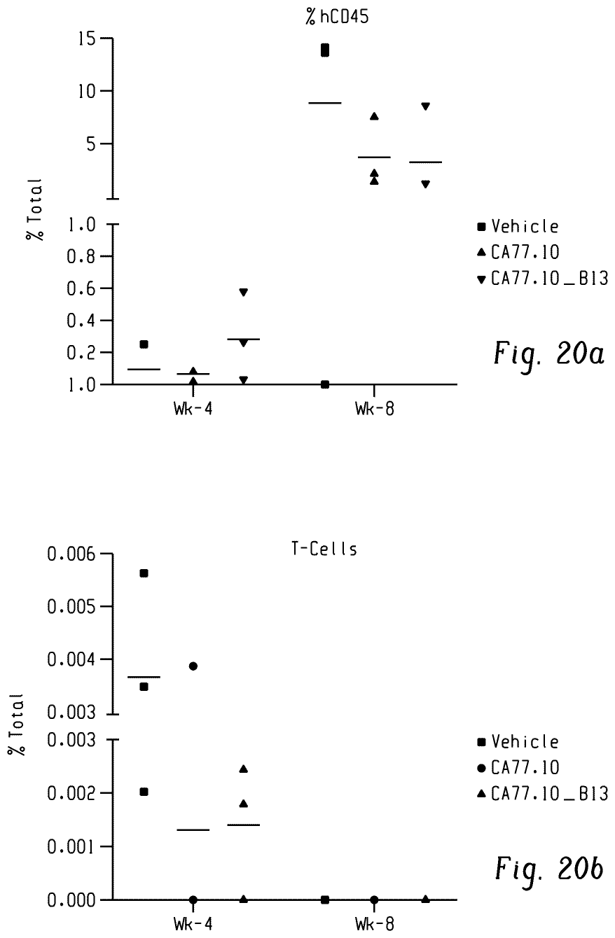

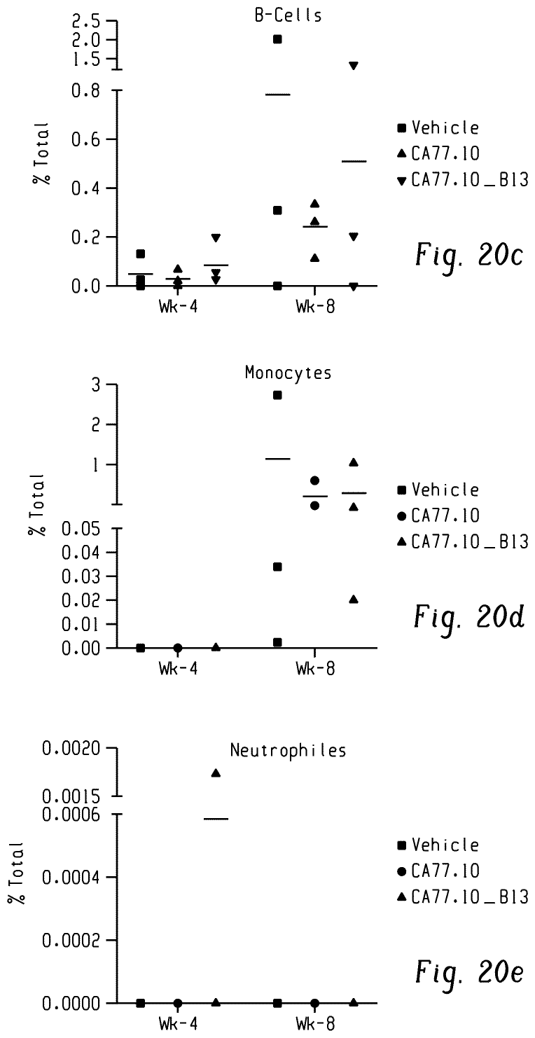

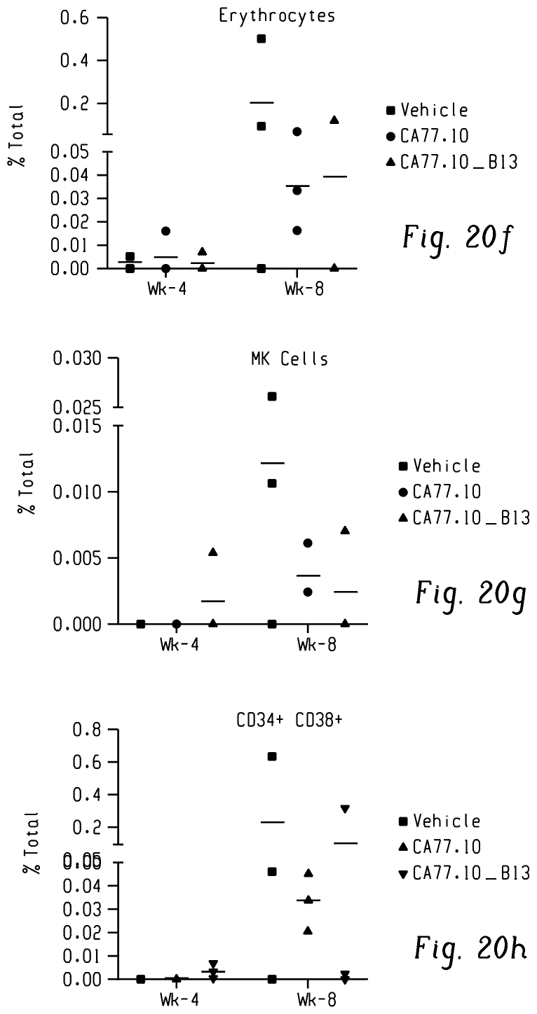

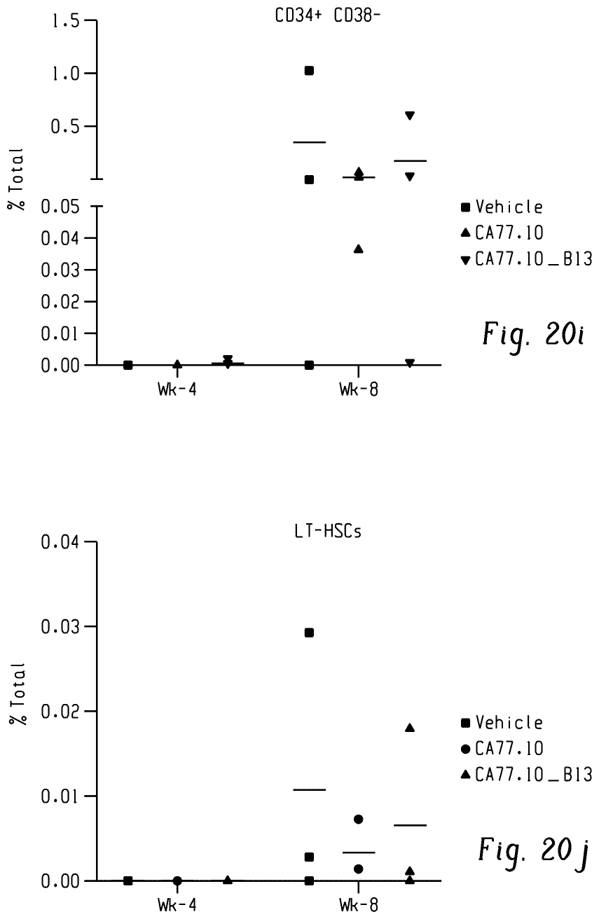

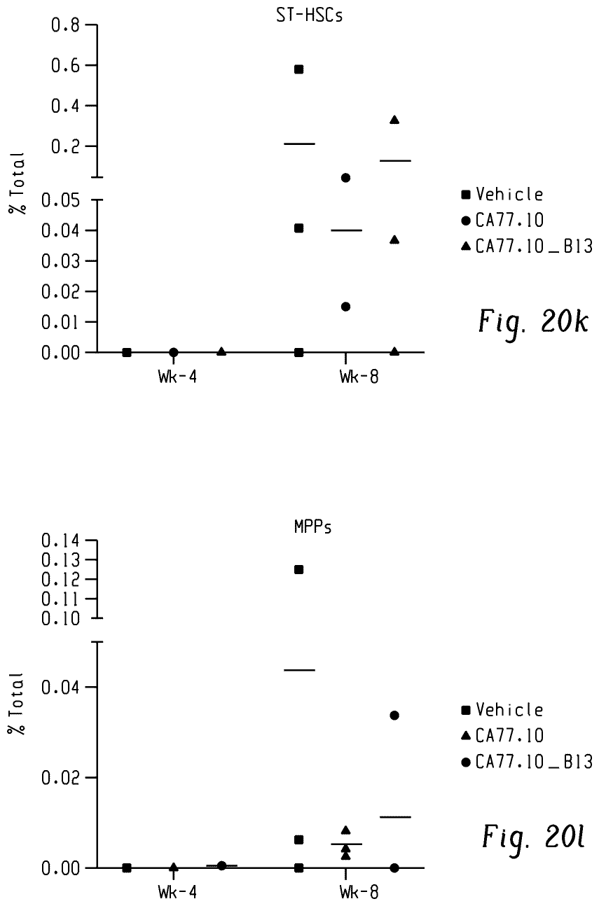

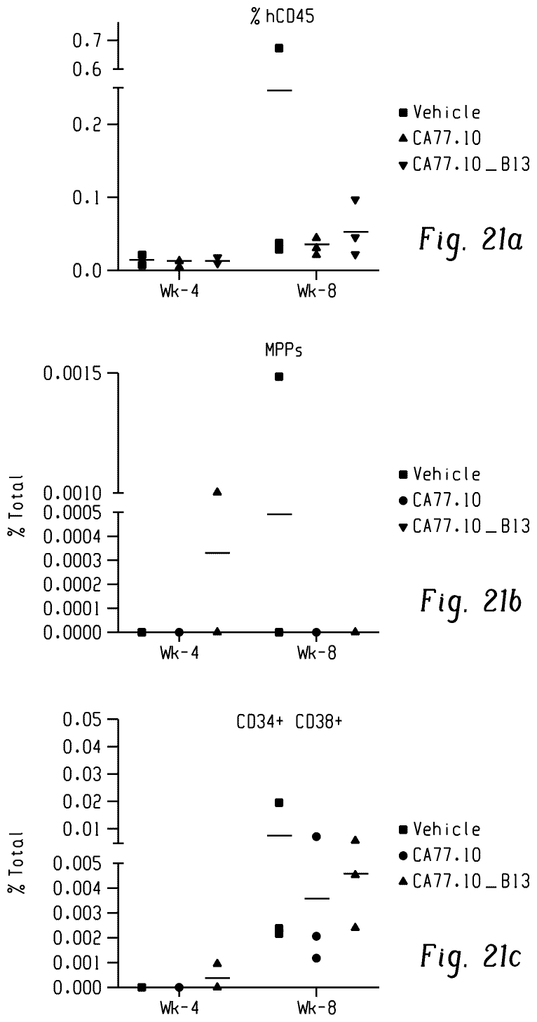

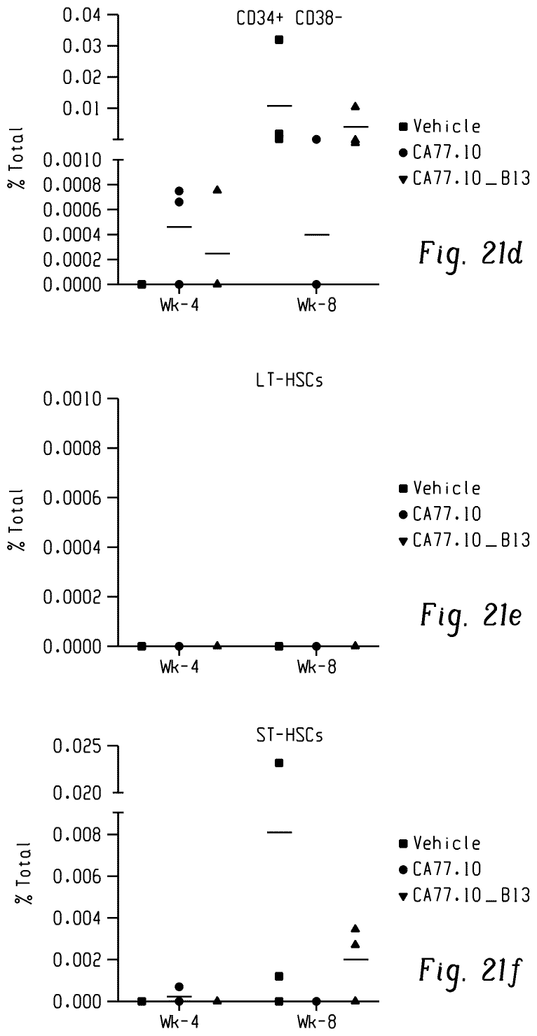

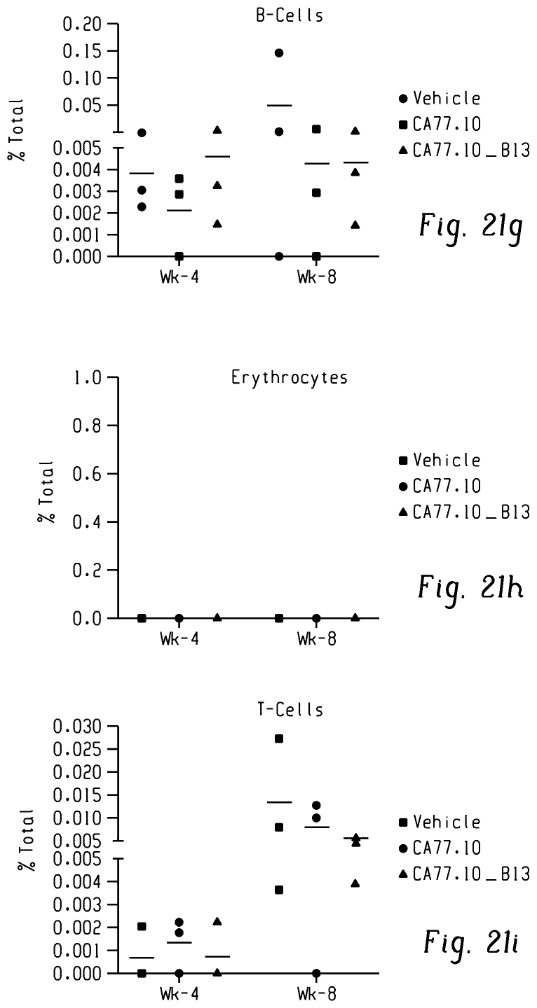

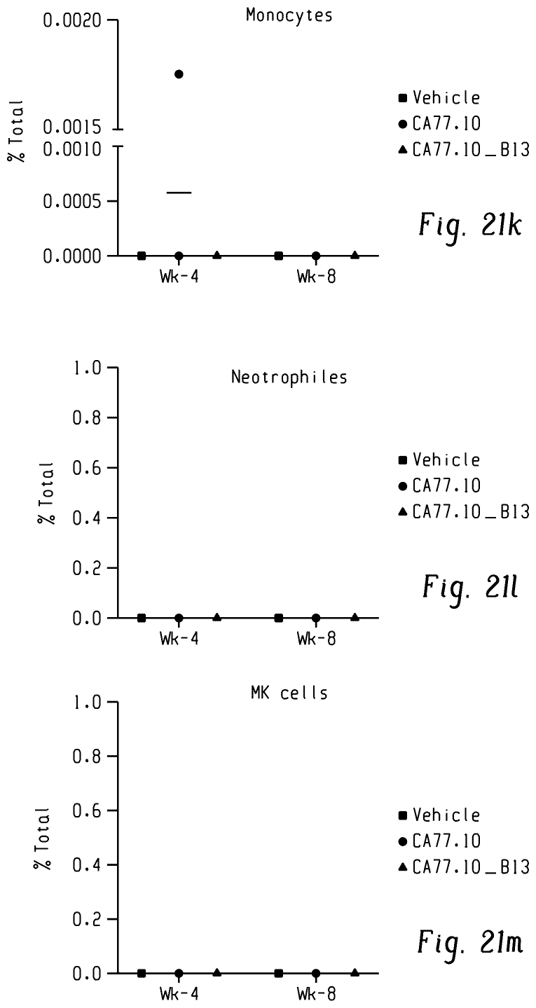

The connection between CMA and fatty acid metabolism through regulation of FADS2 identified in this study, along with the prevention of the age-dependent decline of this metabolic pathway in HSC of old mice with preserved CMA activity (FIG. 4k) and the fact that polyunsaturated fatty acids generated by FADS2 activity also decrease with age in human blood from 250 heathy blood donor volunteers (FIG. 14a) motivated us to also test the effect of the CMA activator on human HSC cells. Addition of CA to CD34+ hematopoietic stem and progenitor cells (HSPC) from old donors (>59 years) markedly increased multi-lineage potent HSPC and sustained overall cell output upon long term culture (FIG. 4t,u). These findings support that pharmacological activation of CMA could be an effective strategy to improve and restore function of aged HSC.

We claim:

1. A method of expanding a population of hematopoietic stem cells (HSCs) while maintaining the population in an undifferentiated state comprising culturing the population of HSCs in the presence of a CMA activator for a period of time until a sufficient number of HSCs is obtained thereby producing an expanded HSC population.

2. The method of claim 1, wherein the expanded HSC population expresses hL2A.

3. A method for suppressing the differentiation of a hematopoietic stem cell (HSC) comprising contacting the HSC with a CMA activator.

4. The method of claim 3, wherein the CMA is administered to a subject.

5. The method of claim 4, wherein the CMA is administered to the subject prior to, contemporaneously with, or after, the subject receives an HSC transplant.

6. A method for treating or preventing a disease or disorder resulting from a deficiency or dysfunction of hematopoietic stem cells (HSCs), comprising administering to a subject in need thereof an effective amount of a CMA activator and/or the expanded HSC population of claim 1.

7. A method of treating or preventing a disease of disorder of the hematopoietic system comprising administering to a subject in need thereof an effective amount of a CMA activator and or the expanded HSC population of claim 1.

8. The method of claim 6, wherein said administration enteral or parental.

9. The method of claim 8, wherein administration of the expanded HSC population is intravenous.

10. The method of claim 8, wherein the CMA activator is administered orally.

11. The method of claim 6, where the disease or disorder is a red blood cell disorder, anemia, congenital dyserythropoietic anemia, congenital sideroblastic anemia, G6PD deficiency, hemochromatosis, hemolytic anemia, hemolytic disease of the newborn, hydrops fetalis, iron deficiency anemia, iron-refractory iron deficiency anemia (IRIDA syndrome), megaloblastic anemia (including pernicious anemia), pyruvate kinase PK) deficiency, sickle cell disease, spherocytosis, thalassemia, white blood cell disorders, cyclic neutropenia, severe congenital neutropenia (Kostmann syndrome), chronic granulomatous disease, leukocyte adhesion deficiency, myeloperoxidase deficiency, bone marrow failure syndromes, aplastic anemia, congenital amegakaryocytic thrombocytopenia, Diamond-Blackfan anemia, dyskeratosis congenita, Fanconi anemia, myelodysplastic syndrome (MDS), Pearson syndrome, Shwachman-Diamond syndrome, thrombocytopenia absent radius, bleeding disorders, hemophilia, hypofibrinogenemia and dysfibrinogenemia, platelet function disorders thrombocytopenia, Von Willebrand disease, thrombosis, anticoagulation disorders, antithrombin deficiency, factor V leiden, protein C deficiency, protein S deficiency, prothrombin gene mutation, stroke, thrombosis, autoimmune blood cell disorders, autoimmune hemolytic anemia, Evans syndrome, immune thrombocytopenia (IPT), a myeloproliferative neoplasm (MPN), or polycythemia.

12. The method of claim 11 wherein the MPNs is polycythemia vera (PV), essential thrombocythemia (ET), myelofibrosis, juvenile myleomonocytic leukemia (JMML), chronic neutrophilic Leukemia, or chronic eosinophilic leukemia/hypereosinophilic syndrome (HES), mast cell disease, chronic myelogenous leukemia, or acute myeloid leukemia (AML).

13. The method of claim 12, wherein the myelofibrosis is primary myelofibrosis (PMF).

14. A method of expanding a population of blood cells comprising culturing the population of blood cells in the presence of a CMA activator for a period of time until a significant increase in the number of blood cells is obtained, thereby expanding the population of blood cells.

15. The method of claim 14, wherein the blood cell is a red blood cell or a white blood cell.

16. The method of claim 15, wherein the blood cell is white blood cell, and the white blood cell is a leukocyte.

17. The method of claim 16, wherein the leukocyte is a lymphocyte.

18. The method of claim 17, where the lymphocyte is a B-cell or a T-cell.

19. The method of claim 18, wherein the T-cell is a CAR-T cell.

20. The method of claim 6, wherein the CMA activator is a compound of Formula I, Formula II, or Formula III or a pharmaceutically acceptable salt thereof.
